SEQUENCE LISTING (12 sequences)

SEQ ID NO: 1 - protein, 9 aa, synthetic construct
  VARIANT 1..9: note = CDR H1
YTFTDYYIH

SEQ ID NO: 2 - protein, 14 aa, synthetic construct
  VARIANT 1..14: note = CDR H2
WMAWISPHTGGTIY

SEQ ID NO: 3 - protein, 14 aa, synthetic construct
  VARIANT 1..14: note = CDR H3
RGPDDWNDGDAFDI

SEQ ID NO: 4 - protein, 14 aa, synthetic construct
  VARIANT 1..14: note = CDR L1
QSVLYSSNNENFLA

SEQ ID NO: 5 - protein, 11 aa, synthetic construct
  VARIANT 1..11: note = CDR L2
LLIYWASTRES

SEQ ID NO: 6 - protein, 8 aa, synthetic construct
  VARIANT 1..8: note = CDR L3
QQYYSTPI

SEQ ID NO: 7 - protein, 116 aa, synthetic construct
EVQLVESGGGLVQPGGSLRLSCAASGFNIKDTYIHWVRQAAPGKGLEWVARIYPTNGYTRYADSVKGRFTISADTSKNTAYLQMNSLRAEDTAVYYCSRWGGDGFYAMDYWGQGTL

SEQ ID NO: 8 - protein, 106 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEI

SEQ ID NO: 9 - DNA, 19 bp, synthetic construct
  source 1..19: mol_type = other DNA
  misc_feature 1..19: note = TRAC
cagggttctggatatctgt

SEQ ID NO: 10 - DNA, 20 bp, synthetic construct
  source 1..20: mol_type = other DNA
  misc_feature 1..20: note = TRBC
cccaccagctcagctccacg

SEQ ID NO: 11 - DNA, 20 bp, synthetic construct
  source 1..20: mol_type = other DNA
  misc_feature 1..20: note = HLA-A2
cctcgtcctgctactctcgg

SEQ ID NO: 12 - protein, 478 aa, synthetic construct
MALPVTALLLPLALLLHAARPEQKLISEEDLEISEVQLVESGGGLVQPGGSLRLSCAASGYTFTDYYIHWVRQAPGKGLEWMAWISPHTGGTIYADSVKGRFTISADTSKNTAYLQMNSLRAEDTAVYYCARGPDDWNDGDAFDIWGQGTLVTVSSGGGGSGGGGSGGGGSSDIQMTQSPSSLSASVGDRVTITCKSSQSVLYSSNNENFLAWYQQKPGKAPKLLIYWASTRESGVPSRFSGSRSGTDFTLTISSLQPEDFATYYCQQYYSTPITFGQGTKVEIKESKYGPPCPPCPMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0358833 A1
(43) Pub. Date: Oct. 31, 2024

(54) HLA-A2-SPECIFIC REGULATORY T CELLS

(71) Applicant: The Regents of the University of California, Oakland, CA (US)

(72) Inventors: Qizhi TANG, Oakland, CA (US); Yannick D. MULLER, Epalinges (CH); Leonardo M.R. FERREIRA, Oakland, CA (US); Emilie RONIN, Oakland, CA (US); Johanna C. WAGNER, Wuerzburg (DE); Yani PENG, Oakland, CA (US)

(21) Appl. No.: 18/682,764

(22) PCT Filed: Aug. 9, 2022
(86) PCT No.: PCT/US22/74720

§ 371 Date: Feb. 9, 2024

Related U.S. Application Data
(60) Provisional application No. 63/231,615, filed on Aug. 10, 2021.

(51) Int. Cl. A61K 39/00; A61P 37/06; C07K 14/725; C07K 16/28; C12N 5/0783

(52) U.S. Cl. CPC A61K 39/46434; A61K 39/4611; A61K 39/4621; A61K 39/4631; A61P 37/06; C07K 14/7051; C07K 16/2833; C12N 5/0637; C07K 2317/24; C07K 2317/33; C07K 2317/622; C12N 2510/00

(57) ABSTRACT

The present disclosure relates to chimeric-antigen-receptor regulatory T cells (CAR-Tregs) that include an exogenous nucleic acid encoding a chimeric antigen receptor (CAR) comprising the complementarity determining regions (CDRs) of a human anti-HLA-A2 antibody grafted onto an antibody scaffold, a hinge domain, a transmembrane domain; and an intracellular domain, where the CAR-Treg expresses the CAR on the cell surface. Vectors and methods of making and using such CAR-Tregs are disclosed as well.

### CROSS REFERENCE TO RELATED APPLICATIONS

This application claims priority to U.S. Provisional Patent Application No. 63/231,615, filed Aug. 10, 2021, the disclosure of which are incorporated by reference herein in their entireties, including any drawings.

### INCORPORATION OF THE SEQUENCE LISTING

This application contains a Sequence Listing, which is hereby incorporated by reference in its entirety. The accompany Sequence Listing text file, named 048536-706001WO.XML,” was created on Aug. 7, 2022 and is 16 KB.

### FIELD

The invention relates to chimeric antigen receptors (CAR) and their components for the regulation of an immune response, for example for transplantation immunology.

### BACKGROUND

Organ transplantation remains an important, and often the only, therapy option for patients with end-stage organ dysfunction. However, treatment necessitates life-long immunosuppression to prevent allograft rejection. Unfortunately, immunosuppressive regimens can be directly toxic to both host and transplanted organs, and also increase patient susceptibilities to cancer development and infections. Thus, strategies to reduce the burden of immunosuppression would reduce the side effects of the current medication. One mechanism is the induction of immune tolerance.

Regulatory T cells (Tregs) are a small subset of CD4⁺ T cells that are key for maintaining self-tolerance and preventing autoimmune disease (1). A plethora of preclinical models have shown that the infusion of Tregs can suppress graft rejection and promote transplant tolerance (2). Several phase I/II clinical studies using Tregs have been reported (3, 4). The ONE Study is the largest coordinated international study of regulatory cell therapies in kidney transplantation. The study includes 28 patients who received Treg therapy in 4 non-randomized single-arm phase I/IIa trials. The results demonstrated feasibility, safety, and potential benefit of Treg-based therapies to reduce the burden of immunosuppression (5). While a significant fraction of Tregs in the polyclonal pool can react to allogeneic donor antigens, data from preclinical models show that donor-reactive Tregs are more effective than polyclonal Tregs in promoting transplant tolerance (6). Unfortunately, donor alloantigen-reactive Tregs may be functionally altered or induced to migrate out of the peripheral blood following transplantation, thus limiting the frequency of alloantigen-reactive clones within polyclonal Treg products and thereby posing challenges for consistent expansion of donor-reactive Tregs (2).

Redirecting Treg alloantigen reactivity using an alloantigen-specific chimeric antigen receptor (CAR) may present a more reliable approach to enhancing therapeutic Treg donor reactivity and potency (7-9). Previous studies have shown that a CAR, consisting of a mouse anti-HLA-A2 (A2) antibody-derived single chain variable fragment (scFv) coupled to a CD28-ζ signaling domain, could be introduced in human Tregs using lentivirus (10). However, in all these studies, the CAR constructs were introduced into the Tregs via lentivirus and the engineered Tregs also expressed their endogenous TCR. Lentiviral transduction results in random integration of the CAR construct in the genome that can lead to variable levels of CAR expression, transcriptional silencing, or accidental disruption of important genes. In addition, it was recently observed that CAR^{hi} human T cells exhibited a surprisingly robust proliferative response to anti-CD28 stimulation alone, independent of CAR or TCR engagement, whereas CAR^{lo} T cells did not (14). Thus, lentivirally engineered Tregs may result in heterogeneous CAR expression and unexpected properties of the engineered cells. However, it is unclear whether CAR Tregs can function without the endogenous TCR.

Further, despite the demonstration of allograft protection by A2-CAR Tregs, prior studies have focused on the protection of non-vascularized grafts, whereas most transplanted grafts are vascularized in the clinical setting. In addition, evaluations of A2-CAR Treg function have been complicated by several notable limitations of humanized mouse models. First, NSG mice are severely immunodeficient, and the engraftment of human T cells alone does not recapitulate the full network of immune-cell interactions that contribute to allograft rejection. A fraction of human T cells is also xenoreactive against mouse antigens, leading to systemic inflammation that limits experimental duration and confounds the antigraft immune response. While the study of murine A2-CAR Tregs in a syngeneic mouse recipient avoided these caveats, the use of a single A2-mismatched skin graft is rare and non-vascularized clinical transplant.

The disclosure provided here provides solutions to the problems existing with previous attempts to suppress graft rejection and promote immune tolerance in the context of non-vascularized and vascularized transplant through infusion of Tregs and provides additional benefits as well.

### SUMMARY

In at least one embodiment, the present disclosure provides a chimeric-antigen-receptor regulatory T cell (CAR-Treg) comprising: an exogenous nucleic acid encoding a chimeric antigen receptor (CAR) comprising the complementarity determining regions (CDRs) of a human anti-HLA-A2 antibody grafted onto an antibody scaffold; a hinge domain; a transmembrane domain; and an intracellular domain, wherein the CAR-Treg expresses the CAR on the cell surface.

In at least one embodiment, the antibody scaffold is trastuzumab.

In at least one embodiment, the CAR-Treg expresses FOXP3, CD25, HELIOS, a demethylated Treg-specific demethylated region (TSDR), or a combination thereof.

In at least one embodiment, the hinge domain is an IgG4, CD28, or CD8a hinge domain.

In at least one embodiment, the transmembrane domain is a CD28 transmembrane domain or a CD8 transmembrane domain.

In at least one embodiment, the intracellular domain comprises a signaling domain. In at least one embodiment, the signaling domain comprises a CD3 domain.

In at least one embodiment, the signaling domain comprises a costimulatory domain. In at least one embodiment, the signaling domain further comprises a CD3 domain.

In at least one embodiment, the intracellular domain does not comprise a signaling domain.

In at least one embodiment, the CD3 domain is derived from CD3ζ and the costimulatory domain is derived from the CD28 costimulatory domain.

In at least one embodiment, the CAR-Treg does not express an endogenous TCR.

In at least one embodiment, the exogenous nucleic acid is integrated into the TCR alpha constant (TRAC) locus of the CAR-Treg.

In at least one embodiment, the anti-HLA-A2 antibody is directed to an HLA-A allele selected from A*02:01, A*02:02, A*02:03, A*02:04, A*02:05, A*02:06, A*02:07, A*02:08, A*02:09, A*02:10, A*02:11, A*02:12, A*02:13, A*02:14, A*02:16, A*02:17, A*02:18, A*02:20, A*02:21, A*02:22, A*02:24, A*02:25, A*02:26, A*02:27, A*02:28, A*02:29, A*02:30, A*02:31, A*02:33, A*02:34, A*02:35, A*02:36, A*02:37, A*02:38, A*02:40, A*02:41, A*02:42, A*02:45, A*02:46, A*02:47, A*02:48, A*02:49, A*02:50, A*02:51, A*02:52, A*02:54, A*02:55, A*02:56, A*02:57, A*02:58, A*02:59, A*02:60, A*02:61, A*02:62, A*02:63, A*02:64, A*02:66, A*02:67, A*02:68, A*02:69, A*02:70, A*02:71, A*02:72, A*02:73, A*02:74, A*02:75, A*02:76, A*02:77, A*02:78, A*02:80, A*02:81, A*02:84, A*02:85, A*02:87, A*02:89, A*02:90, A*02:91, A*02:92, A*02:93, A*02:95, A*02:96, A*02:97, A*02:99, A*02:101, A*02:102, A*02:103, A*02:104, A*02:105, A*02:106, A*02:107, A*02:108, A*02:109, A*02:110, A*02:111, A*02:112, A*02:114, A*02:115, A*02:116, A*02:117, A*02:119, A*02:120, A*02:121, A*02:122, A*02:123, A*02:124, A*02:126, A*02:127, A*02:128, A*02:129, A*02:130, A*02:131, A*02:132, A*02:133, A*02:134, A*02:136, A*02:137, A*02:138, A*02:139, A*02:140, A*02:141, A*02:142, A*02:143, A*02:144, A*02:145, A*02:146, A*02:147, A*02:148, A*02:150, A*02:151, A*02:153, A*02:154, A*02:155, A*02:156, A*02:157, A*68:01, A*68:02, A*68:03, A*68:04, A*68:05, A*68:06, A*68:07, A*68:08, A*68:09, A*68:10, A*68:13, A*68:14, A*68:15, A*68:16, A*68:17, A*68:19, A*68:20, A*68:21, A*68:22, A*68:23, A*68:24, A*68:25, A*68:26, A*68:27, A*68:28, A*68:29, A*68:30, A*68:31, A*68:32, A*68:33, A*68:34, A*68:35, A*68:36, A*68:37, A*68:38, A*68:39, A*68:40, A*68:41, A*68:42, A*68:43, A*68:44, A*68:45, A*68:46, A*68:47, A*69:01.

In at least one embodiment, the CAR-Treg suppresses in vitro proliferation of T cells in the presence of HLA-A2+ cells.

In at least one embodiment, the CAR-Treg accumulates in HLA-A2 expressing tissue in vivo.

In at least one embodiment, CAR-Treg suppresses the immune response in the presence of HLA-A2+ and HLA-A28+ cells in vivo.

In at least one embodiment, the present disclosure provides a vector comprising: an exogenous nucleic acid encoding a chimeric antigen receptor (CAR) comprising the complementarity determining regions (CDRs) of an anti-HLA-A2 antibody grafted onto an antibody scaffold; a hinge domain; a transmembrane domain; and an intracellular domain.

In at least one embodiment, the antibody scaffold is human trastuzumab.

In at least one embodiment, the CAR-Treg expresses FOXP3, CD25, HELIOS or a combination thereof.

In at least one embodiment, the hinge domain is an IgG4, CD28, or CD8a hinge domain.

In at least one embodiment, the intracellular domain comprises a signaling domain. In at least one embodiment, the signaling domain comprises a CD3 domain.

In at least one embodiment, the signaling domain comprises a costimulatory domain. In at least one embodiment, the signaling domain further comprises a CD3 domain.

In at least one embodiment, the intracellular domain does not comprise a signaling domain.

In at least one embodiment, the CD3 domain is derived from CD3ζ and the costimulatory domain is derived from the CD28 costimulatory domain.

In at least one embodiment, the vector is a viral vector. In at least one embodiment, the viral vector is a retroviral vector. In at least one embodiment, the retroviral vector is a lentiviral vector.

In at least one embodiment, the vector comprises a nucleic acid that encodes the amino acid sequence of SEQ ID NO:12.

In at least one embodiment, the present disclosure provides a method for generating chimeric-antigen-receptor regulatory T cells (CAR-Tregs) comprising: providing a population of regulatory T cells; and contacting the population of regulatory T cells with one or more vectors of the present disclosure under conditions sufficient for transduction of the vector.

In at least one embodiment, the present disclosure provides a method for inducing immune tolerance for organ transplantation comprising: providing a population of CAR-Tregs of the present disclosure; administering the population of CAR-Tregs to a subject having received an organ transplant.

In at least one embodiment, the organ transplant is a non-vascularized transplant. In at least one embodiment, the non-vascularized transplant is selected from skin and pancreatic islets.

In at least one embodiment, the organ transplant is a vascularized transplant. In at least one embodiment, the vascularized transplant is selected from heart, lung, kidney, liver, pancreas, small intestine, or composite tissue allograft.

In at least one embodiment, said administering comprises administering at least 30 million (3×10⁷) to 500 million (5×10⁸) cells to the subject.

In at least one embodiment, said administration induces immune tolerance to transplanted cells and tissue such that a dose of immunosuppressive drug can be reduced or eliminated.

Each of the aspects and embodiments described herein are capable of being used together, unless excluded either explicitly or clearly from the context of the embodiment or aspect.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows phage-displayed SN607D8 scFv binding to HLA-A2- and HLA-A28-expressing cell lines. The scFv from the anti-HLA-A2 hybridoma SN607D8 was cloned into a phage vector and expressed on the surface of the phage. The HLA-A2⁺HLA-A28⁻ THP-1 cells or the HLA-A2⁻HLA-A28⁺ RPMI 8226 cells were incubated with 10⁷ phages displaying the SN607D8 scFv. The cells were then stained with a biotinylated anti-phage antibody, followed by fluorochrome-conjugated streptavidin. Cell-bound phage was then analyzed using flow cytometry. Cells not incubated with phage or incubated with control phage were similarly stained and included as controls.

FIG. 2A shows the grafting strategy comparing the original VH and VL chain sequences of the SN607D8 hybridoma and of the Herceptin (trastuzumab) 4D5 scaffold. The grafted amino acid sequences are shown. The sequences of SN607D8 and 4D5 HER2 were aligned using the software Jalview and the level of conservation (C) and quality (Q) of each amino acid between SN607D8 and 4D5 sequences were compared. Conservation reflects similarity of the physicochemical properties of amino acid residues. Identical residues are shown as light-yellow columns and residues with more dissimilar physicochemical properties are marked with darker column colors. Quality measures the likelihood of observing a mutation in any particular amino acid residue position (45). CDRs were predicted using Paratome (21).

FIG. 2B shows the conformation of the grafted antibody as predicted with ABodyBuilder (31) and displayed using PyMOL Molecular Graphics System (DeLano Scientific, San Carlos, CA).

FIG. 2C shows EGFRt and MYC-tag expression on Day 6 of culture of human Tregs transduced with the grafted A2-CAR-2A-EGFRt lentivirus.

FIG. 2D shows OneLambda FlowPRA Single HLA Antigen bead panels FL1HD01 and FL1HD02. Percentage relative binding of A2-CAR Tregs to each HLA allele was calculated as described in the Examples. Plotted averages of at least 5 independent experiments are shown. Red coloring indicates HLA allele beads surpassing the 25% binding threshold to be considered binders.

FIG. 2E shows that, on Day 9, A2-CAR Tregs were cultured for another 48 hours alone, with anti-CD3/CD28 beads, or with irradiated (4000 rad) parental A2⁻ K562 or A2-expressing K562 cells. CD25, CD71, ICOS, CTLA-4, and FOXP3 expression were analyzed thereafter using flow cytometry. Abbreviations: scFv, single-chain variable fragment; CDR, complementarity-defining region; A2, HLA-A2; UT, untransduced.

FIG. 2F shows that, on Day 9, A2-CAR or UT T cells were cultured for another 48 hours alone or with dissociated islet cells from 4 allogeneic donors. Expression of CD71 was analyzed thereafter using flow cytometry. The HLA-A and B alleles expressed by the 4 allogeneic donors are indicated above the histograms. For donors 1, 3, and 4, A2-CAR and UT Tregs were used in the assay and for donor 2, A2-CAR and UT CD4⁺ Tconvs are used. Results are a summary of 4 independent experiments using T cells from unrelated healthy donors.

FIG. 3A shows a map of the grafted anti-HLA-A2 (A2) CAR gene fused to a luciferase (luc) or an EGFRt reporter gene.

FIG. 3B shows A2-CAR lentivirus titered on 1×10⁴ Jurkat T cells. CAR surface expression was assessed by MYC-tag staining 72 hours post-transduction.

FIG. 3C shows 0.5×10⁴ A2-CAR transduced Jurkat T cells co-cultured with irradiated (4000 rad) GFP⁺ K562 cells expressing HLA-A2 at a 1:1 ratio for 24 hours. CD69 and CD25 expression were analyzed using flow cytometry. As a control, A2-CAR Jurkat T cells were incubated alone. Abbreviations: P, promoter; H, hinge; TM, transmembrane; ICD, intracellular domain; Luc, luciferase; A2, HLA-A2.

FIG. 4A shows experimental design of human T-cell engineering and expansion.

FIG. 4B shows editing efficiency as measured by CD3 and MYC-tag expression on T cells prior to infusion.

FIG. 4C shows WT NSG or HLA-A2 transgenic NSG mouse islets pictured 48 h after co-culture with A2-CAR T cells (100 IEQ with 1×10⁵ T cells).

FIG. 4D shows luciferase activity over time after infusion of 2×10⁶ A2-CAR T cells in mice that received an A2 transgenic murine islet transplant either under the left kidney capsule or into the spleen.

FIG. 4E shows glycemia monitoring after streptozotocin (STZ) injection, islet transplantation, and T-cell infusion (2×10⁶/mouse). An insulin pellet was placed subcutaneously in mice with partial graft function (blood glucose >200 mg/dl) on Day 10 after transplantation.

FIG. 5 shows human CD4⁺ Tconv cell trafficking and accumulation in NSG mice. 5×10⁶ CFSE-labeled human CD4⁺ Tconv cells were injected i.v. into NSG mice. On Day 2, 7, and 14 after cell transfer, their presence (top row) and proliferation (bottom histograms) in the bone marrow (BM), spleen (Spl), liver (Liv), lungs, and kidneys (Kid) of the recipient mice were analyzed using flow cytometry.

FIG. 6 shows engineering human A2-CAR⁺ TCR^{deficient} Tregs. CD4⁺CD25^{high}CD127^{low} Tregs and CD4⁺CD25^{low}CD127^{high} conventional T cells (Tconv) were sorted using FACS. Thereafter, the T-cell receptor (TCR) was deleted using CRISRPR/Cas9 and the cells were activated with anti-CD3/CD28 beads. Two days later, cells were lentivirally transduced with A2-CAR. CD25, FOXP3, CD3, and MYC-tag expression were measured on Day 9.

FIG. 7A shows trafficking of A2-CAR Tregs to islet grafts. 2×10⁶ A2-CAR CD4⁺ Tconv cells or A2-CAR Tregs (harvested on Day 10 after sorting from peripheral blood) were infused into mice that had been stably transplanted with HLA-A2 transgenic mouse islets. Luciferase activity of A2-CAR CD4⁺ Tconv cells or A2-CAR Tregs over time is shown.

FIG. 7B shows trafficking of A2-CAR Tregs to islet grafts. Glycemia was monitored over time after cell infusion.

FIG. 7C shows trafficking of A2-CAR Tregs to islet grafts. Tregs were edited with CRISPR/Cas9 ribonucleoprotein (RNP) complexes targeting the TRBC locus after cell sorting, activated with anti-CD3/28 beads, and transduced with A2-CAR linked to a luciferase reporter gene by a 2A self-cleaving peptide two days later. On Day 9, Tregs were re-stimulated with HLA-A2⁺ stimulated B cells (sBCs) for another 5 days and were thereafter injected in diabetic mice (2×10⁶/animal) transplanted with HLA-A2⁺ human islets. In parallel, polyclonal Tregs were activated with anti-CD3/28 beads, transduced with a lentivirus expressing a luciferase reporter gene alone, and restimulated with anti-CD3/28 beads on Day 9 and injected on Day 14 in diabetic mice (2×10⁶/animal) transplanted with HLA-A2⁺ human islets.

FIG. 7D shows trafficking of A2-CAR Tregs to islet grafts. Editing efficiency was measured by MYC-tag and CD3 surface expression in two independent donors. Treg purity was assessed by FOXP3 and HELIOS expression in the same donors.

FIG. 7E shows trafficking of A2-CAR Tregs to islet grafts. Luciferase activity of A2-CAR Tregs and polyclonal Tregs (transduced with a lentivirus expressing a luciferase reporter alone) over time.

FIG. 8A shows precision engineering of an A2-CAR into the TRAC locus of human Tregs. Homology-directed repair-mediated integration into the TRAC locus. The A2-CAR template was inserted using AAV6 transduction after electroporation of two CRISPR/Cas9 ribonucleoprotein (RNP) complexes targeting the TRAC and HLA-A loci.

FIG. 8B shows precision engineering of an A2-CAR into the TRAC locus of human Tregs. Representative flow cytometry of the editing efficiency measured 10 days later in 3 independent experiments. CD3, MYC-tag, and HLA-A2 surface expression is shown.

FIG. 8C shows precision engineering of an A2-CAR into the TRAC locus of human Tregs. Treg fold-expansion and percentage of MYC-tag⁺ Tregs over time. Fitted line plots are shown.

FIG. 8D shows precision engineering of an A2-CAR into the TRAC locus of human Tregs. Fourteen days after activation, FOXP3 and HELIOS expression were assessed on edited Tregs and compared to that of unedited T cells and unedited Day 9 stimulated Tregs (unEd sTregs).

FIG. 8E shows precision engineering of an A2-CAR into the TRAC locus of human Tregs. The same cells were co-cocultured with or without irradiated (4000 rad) HLA-A2⁺ NALM6 cells. CD71 expression was assessed 48 hours later.

FIG. 8F shows precision engineering of an A2-CAR into the TRAC locus of human Tregs. In vitro suppression assays were performed using HLA-A2⁺ stimulated B cells (sBCs) as stimulator cells, A2-CAR⁺ TCR^{deficient} CD4⁺ T cells as responder cells (0.05×10⁶ cell/96 well) and A2-CAR⁺ TCR^{deficient} Tregs at different ratios. After 3 days, 0.5 uCi/well of 3[H] thymidine (Perkin Elmer, Waltham, MA) was added for the final 16 h of culture. Proliferation was assessed by ³[H] thymidine incorporation (counts per minute—cpm). Two-way ANOVA was used to determine the statistical significance of the difference. Data corresponds to the cells infused in FIG. 9. Similar results were obtained with 3 independent donors. ** p<0.01; ***p<0.001. Abbreviations: Teff; CD4⁺ T effector cells; A2-Treg, A2-CAR⁺TCR^{deficient} Treg; unEd sTreg, unedited Day 9 stimulated Tregs; A2-Teff; A2-CAR⁺TCR^{deficient} Teff sBC, stimulated B cells; CPM, counts per minute.

FIG. 9A shows A2-CAR Tregs confer protection against graft-versus-host disease. 2.5×10⁶ AAV CRISPR-edited A2-CAR⁺ TCR^{deficient} Tregs were co-injected with 5×10⁶ A2 negative or positive PBMC in wild type or A2 transgenic immunodeficient NSG mice. PBMCs and Tregs were injected into contralateral retro-orbital plexus.

FIG. 9B shows A2-CAR Tregs confer protection against graft-versus-host disease. Overall survival of mice that received PBMCs (A2 positive or negative, NSG A2 positive or negative) or PBMCs and A2-CAR⁺ TCR^{deficient} Tregs, excluding the condition where Tregs remained unstimulated (group 1-2).

FIG. 9C shows A2-CAR Tregs confer protection against graft-versus-host disease. Overall survival of mice treated with A2-CAR⁺ TCR^{deficient} Tregs comparing NSG PMBC (group 1.2, round), A2-NSG PBMC (group 2-2, triangle), NSG A2⁺ PBMC (group 3-2, inverted triangle, n=6), and A2-NSG A2⁺ PBMC (group 4-2, square). Log-rank (Mantel-Cox) test was used for statistical analysis * p<0.05, **p<0.01.

FIG. 10A shows in vitro A2-CAR Treg activation by PBMCs from an HLA-A2⁺ donor. HLA-A2 expression on PBMCs from two donors used for immune reconstitution in NSG mice in FIG. 5.

FIG. 10B shows A2-CAR Tregs were co-cultured for 48 hours at 1:1 ratio with the PBMCs of each donor. CD71 expression was analyzed using flow cytometry.

FIG. 11A shows blood monitoring of immune reconstitution in xenogeneic graft versus-host disease. Representative staining of human CD8⁺, human CD4⁺ and hCD4⁺ A2^{neg}FOXP3⁺ subpopulations after dead cell (Ghost fixable viability dye positive) exclusion. Representative example of mice from each group (1-1, 1-2, 2-1, 2-2, 3-1, 3-2, 4-1, 4-2).

FIG. 11B shows blood monitoring of immune reconstitution in xenogeneic graft versus-host disease. Pie charts showing the mean percentage of human CD4⁺ (orange) and CD8⁺ (blue) cells in the peripheral blood of mice 7 (n=4-6), 14 (n=2-5) and 21 days (n=2-4) after injection. The numbers below each pie chart represent the mean±standard deviation of the combined percentage of human T cells (hCD4⁺ and hCD8⁺). Abbreviations: A2, HLA-A2

FIG. 12A shows a schematic of murine anti-human HLA-A2 CAR construct design. scFv, single-chain variable fragment; TMD, transmembrane domain; ICD, intracellular domain.

FIG. 12B shows workflow for CAR-T cell generation and characterization.

FIG. 12C shows Myc MFI of retrovirally transduced Tconv and Tregs assessed on day 6. Plotted data indicate the mean and standard deviation of n=5 (Tconv) and n=15 (Treg) independent experiments (929.0±158.61, n=5 and 1238±732.21, n=15; p=0.3867). Flow cytometry plots for CAR surface expression and FOXP3 expression of stably transduced Tconvs and Tregs sorted based on EGFRt expression. Plots are representative of 5 independent experiments. Statistics were determined via an unpaired t test; n.s., not statistically significant.

FIG. 13A shows A2-specific in vitro activation of CAR+ T cells and A2-specific in vitro suppression of CAR+ Tregs. CD69 and CD25 expression profiles for CAR+ Tconv or CAR+ Tregs as determined by flow cytometry 48 or 72 h following co-incubation with wild-type or A2-expressing K562 cells. Plots are representative of n=4 or n=5 independent experiments for Tconv or Treg, respectively.

FIG. 13B shows A2-specific in vitro activation of CAR+ T cells and A2-specific in vitro suppression of CAR+ Tregs. CellTrace Violet (CTV) dye dilution and percentage of CAR+ Tregs that have undergone at least one cell division by 96 h following co-incubation with irradiated A2-negative or A2-expressing B6 splenocytes. Plotted data indicate the mean and standard deviation of 3 independent experiments. The proliferation significantly increased from 51.57% to 81.13% (**p=0.0047, n=3).

FIG. 13C shows A2-specific in vitro activation of CAR+ T cells and A2-specific in vitro suppression of CAR+ Tregs. A2-CAR Treg-mediated suppression of B6.TCR75+ Tg CD4+ responder cells in the presence of the irradiated A2-expressing B6 splenocytes and absence of TCR stimulation. Data are representative of 2 independent experiments. (****p<0.0001, n=2).

FIG. 13D shows A2-specific in vitro activation of CAR+ T cells and A2-specific in vitro suppression of CAR+ Tregs. TCR-mediated suppression of B6.TCR75+ Tg CD4+ responder cells in the presence of anti-CD3 and A2-negative B6 splenocytes (p=0.6505, n=2). Statistics were determined via Two-Way ANOVA; **p<5E-3, ****p<0.0001.

FIG. 14A shows prolongation of allograft survival in the single A2-mismatch heterotopic heart transplantation model. A schematic of single A2-mismatch heterotopic heart transplant model is shown. A2-expressing hearts from B6.A2 donor mice were transplanted into B6.WT recipient mice 48 h prior to retro-orbital injection of 1×10⁶, 2×10⁶, or 4×10⁶ A2-CAR or 1×10⁶ untransduced, polyclonal Tregs.

FIG. 14B shows prolongation of allograft survival in the single A2-mismatch heterotopic heart transplantation model. Graft survival curve in mice that received syngeneic graft (n=3) or HLA-A2 single mismatched graft with no treatment (n=7) or along with 1×10⁶ A2-CAR (CAR+, n=4) or 1×10⁶ untransduced Tregs (poly, n=4). Significant prolongation of A2-single mismatched grafts when 1×10⁶ A2-CAR Tregs were injected (*p=0.0144) is shown.

FIG. 14C shows prolongation of allograft survival in the single A2-mismatch heterotopic heart transplantation model. Transplanted heart palpation score in the 100 days post transplantation for syngeneic transplants (n=3) or A2-single mismatched heart transplants with no treatment (n=7) or with untransduced, polyclonal (n=4) or A2-CAR Tregs (n=4) is shown.

FIG. 14D shows prolongation of allograft survival in the single A2-mismatch heterotopic heart transplantation model. H&E stain of a syngeneic heart transplant (day 100 post HTx) or on the day of rejection for a single A2-mismatched heart transplant with no treatment (day 15 post HTx), with 1×10⁶ untransduced, polyclonal Tregs (day 25 post HTx) or with 1×10⁶ A2-CAR Tregs (day 100 post HTx).

FIG. 14E shows prolongation of allograft survival in the single A2-mismatch heterotopic heart transplantation model. Graft survival curve following injection of 1×10⁶ (n=4), 2×10⁶ (n=4), or 4×10⁶ (n=4) A2-CAR Tregs (p=0.6617) is shown.

FIG. 14F shows prolongation of allograft survival in the single A2-mismatch heterotopic heart transplantation model. Transplanted heart palpation score in the 100 days post transplantation for A2-single mismatched heart transplants with no treatment or with 1×10⁶ (n=4), 2×10⁶ (n=4), or 4×10⁶ (n=4) A2-CAR Tregs is shown.

FIG. 15A shows prolongation of allograft survival in the haplo-mismatch heterotopic heart transplantation model. Schematic of combined A2- and MHC-mismatch model. A2.F1-expressing hearts from B6.A2×BALB/c donor mice were transplanted into B6.WT recipient mice 24 h prior to retro-orbital injection of 1×10⁶ A2-CAR Tregs is shown. Rapamycin-treated mice received i.p. administration for the first 9 days with or without 1×10⁶ injected on day 7 post transplantation.

FIG. 15B shows prolongation of allograft survival in the haplo-mismatch heterotopic heart transplantation model. Graft survival curve in mice that received a haplo-mismatched graft without treatment (no therapy, n=4), rapamycin only (rapamycin, n=4), A2-CAR Treg infusion only (CAR+ Tregs, n=3), and combination treatment (rapamycin+A2-CAR Tregs, n=4). A2-F1 allografts rejected within 11 days and treatment with 1×10⁶ A2-CAR Tregs prolonged graft survival, however, only by 3 days (*p=0.0207). Treating with rapamycin and A2-CAR Tregs significantly prolonged graft survival both compared to the rapamycin treatment as well as no treatment (*p=0.0266 and *p=0.01, respectively).

FIG. 15C shows prolongation of allograft survival in the haplo-mismatch heterotopic heart transplantation model. Transplanted heart palpation score in the 100 days post transplantation for haplo-mismatched transplants with no treatment (n=4), rapamycin only (n=4), Treg infusion only (n=3), and combination treatment (n=4) is shown.

FIG. 15D shows prolongation of allograft survival in the haplo-mismatch heterotopic heart transplantation model. H&E stain on the day of rejection of a haplo-mismatched heart transplant with no therapy (day 11 post HTx), A2-CAR Tregs (day 13 post HTx), rapamycin treatment (day 26 post HTx) and a combination therapy of A2-CAR Tregs and rapamycin (day 100 post HTx) is shown.

### DETAILED DESCRIPTION

The present disclosure relates generally to regulatory T cells expressing chimeric antigen receptors (CAR-Tregs) containing specific antigen binding domains and signaling components for the regulation of an immune response in the context of organ transplantation. This is because in studies of many existing Treg based therapies, it has been found that donor-reactive Tregs are more effective than polyclonal Tregs in promoting transplant tolerance (6). However, donor alloantigen-reactive Tregs may be functionally altered or induced to migrate out of the peripheral blood following transplantation, thus limiting the frequency of alloantigen-reactive clones within polyclonal Treg products and thereby posing challenges for consistent expansion of donor-reactive Tregs (2). Further, in many studies, the CAR constructs were introduced into the Tregs via lentivirus and the engineered Tregs also expressed their endogenous TCR. This resulted in variable levels of CAR expression, transcriptional silencing, and accidental disruption of important genes. Thus, the CAR constructs of the disclosure generally include an HLA-A2 antigen-specific binding region, a transmembrane region, and an intracellular region and are introduced into Tregs using site-specific integration at the site of the endogenous TCR. Tregs expressing the CAR constructs described herein are demonstrated to be useful in controlling the immune response to both non-vascularized and vascularized transplants.

CAR-Tregs can be used as programmable regulatory cells for adoptive cell therapy, engineered to specifically shape the immune response in the context of organ transplantation. Accordingly, embodiments of the present disclosure are directed to chimeric receptors with antigen binding regions that are capable of inducing immune tolerance, for use in cell therapies, including, but not limited to, antigen-specific cell therapies.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### I. Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Compositions or methods “comprising” or “including,” or any grammatical variant thereof, one or more recited elements can include other elements not specifically recited. For example, a composition that includes antibody can contain the antibody alone or in combination with other ingredients.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

As used herein, and unless otherwise specified, a “therapeutically effective amount” or a “therapeutically effective number” of an agent is an amount or number sufficient to provide a therapeutic benefit in the treatment or management of a disease, e.g., transplant rejection, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208 (2005).

A “transplant,” as used herein, refers to cells, tissue, or an organ that is introduced into a subject. The source of the transplanted material can be cultured cells, cells from another subject, or cells from the same subject (e.g., after the cells are cultured in vitro). Exemplary organ transplants are kidney, liver, heart, lung, and pancreas. An exemplary tissue transplant is islets. An exemplary cell transplant is allogeneic hematopoietic stem cell transplantation.

The term “exogenous” refers to any material introduced from or produced outside an organism, cell, tissue or system.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “linker”, also referred to as a “spacer” or “spacer domain” as used herein, refers to an amino acid or sequence of amino acids that that is optionally located between two amino acid sequences in a fusion protein of the invention.

The term “biological sample” or “sample” refers to any solid or liquid sample isolated from an individual or a subject. For example, it can refer to any solid (e.g., tissue sample) or liquid sample (e.g., blood) isolated from an animal (e.g., human), such as, without limitations, a biopsy material (e.g., solid tissue sample), or blood (e.g., whole blood). Such sample can be, for example, fresh, fixed (e.g., formalin-, alcohol- or acetone-fixed), paraffin-embedded or frozen prior to an analysis. In an embodiment, the biological sample is obtained from a tumor (e.g., a pancreatic cancer). A “test biological sample” is the biological sample that has been the subject of analysis, monitoring, or observation. A “reference biological sample,” containing the same type of biological sample (e.g., the same type of tissues or cells), is a control for the test biological sample.

As used herein, an “individual” or a “subject” includes animals, such as human (e.g., human individuals) and non-human animals. In some embodiments, an “individual” or “subject” is a patient under the care of a physician. Thus, the subject can be a human patient or an individual who has, is at risk of having, or is suspected of having a disease of interest (e.g., graft versus host disease) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### II. Compositions of the Disclosure

As described in greater detail below, one aspect of the present disclosure relates to regulatory T cells (Tregs) engineered to site-specifically express chimeric antigen receptors (CARs) that include an antigen binding domain directed to transplant antigen HLA-A2. Also provided, in other related aspects of the disclosure, are nucleic acids encoding the CARs as disclosed herein, recombinant immune cells expressing the CARs as disclosed herein, and pharmaceutical compositions containing the nucleic acids and/or recombinant cells as disclosed herein.

### Chimeric Antigen Receptors (CARs)

The present disclosure is based, inter alia, on redirected human Tregs to transplant antigen HLA-A2 using a chimeric antigen receptor (CAR), while deleting the Treg's endogenous T cell receptor (TCR), thus creating Tregs with a single specificity to HLA-A2 on transplanted tissue. Tregs expressing these CARs are shown to be useful in the context of both non-vascularized and vascularized transplant immune responses.

The recombinant receptors generally comprise an antigen-specific binding region, a transmembrane region, and an intracellular region. The antigen-specific binding region of the CAR of the disclosure includes the complementarity determining regions (CDRs) of a human anti-HLA-A2 antibody grafted on to an antibody scaffold.

HLA-A2 is a group of human leukocyte antigen (HLA) proteins, including cell surface proteins, encoded by the HLA-A*02 allele family at the HLA-A locus of the HLA gene complex. HLA proteins encompassed by the terms “HLA-A2” include HLA proteins identified as belonging to the HLA-A*02 antigen type by serological testing or genotyping. Additional names for the HLA-A*02 antigen type include “HLA-A2”, HLA-A02” and “HLA-A*2”. Different naming systems have been developed which identify HLA proteins encoded by this family of alleles including the HLA naming system developed in 2010 by the WHO Committee for Factors of the HLA System. The terms “HLA-A2” and “A2” refer to HLA proteins encoded by alleles having designations according to this naming system which begin with “HLA-A*02:”, including but not limited to designations which begin with “HLA-A*02:01”, “HLA-A*02:02”, “HLA-A*02:03”, “HLA-A*02:04”, “HLA-A*02:05”, “HLA-A*02:06”, “HLA-A*02:07”, “HLA-A*02:08”, “HLA-A*02:09”, “HLA-A*02:10”, and “HLA-A*02:11”. In addition to the numerical digits which follow “HLA-A*02:”, the allele designations may also contain an upper case letter, including but not limited to upper case letters “P” and “G” (e.g., HLA-A*02:01P or HLA-A*02:01:01G). The allele designations which begin with “HLA-A*02:” followed by 2, 3, or 4 additional numerical digits may constitute the complete designation or a beginning portion of the designation. The allele designations may be italicized. The terms “HLA-A2” and “A2” also refer to HLA proteins identified with designations which begin with “HLA-A*02:” according to this naming system, including but not limited to the designations “HLA-A*02:01”, “HLA-A*02:02”, “HLA-A*02:03”, “HLA-A*02:04”, “HLA-A*02:05”, “HLA-A*02:06”, “HLA-A*02:07”, “HLA-A*02:08”, “HLA-A*02:09”, “HLA-A*02:10”, and “HLA-A*02:11”.

In certain embodiments, the anti-HLA-A2 antibody is directed to an HLA-A allele selected from A*02:01, A*02:02, A*02:03, A*02:04, A*02:05, A*02:06, A*02:07, A*02:08, A*02:09, A*02:10, A*02:11, A*02:12, A*02:13, A*02:14, A*02:16, A*02:17, A*02:18, A*02:20, A*02:21, A*02:22, A*02:24, A*02:25, A*02:26, A*02:27, A*02:28, A*02:29, A*02:30, A*02:31, A*02:33, A*02:34, A*02:35, A*02:36, A*02:37, A*02:38, A*02:40, A*02:41, A*02:42, A*02:45, A*02:46, A*02:47, A*02:48, A*02:49, A*02:50, A*02:51, A*02:52, A*02:54, A*02:55, A*02:56, A*02:57, A*02:58, A*02:59, A*02:60, A*02:61, A*02:62, A*02:63, A*02:64, A*02:66, A*02:67, A*02:68, A*02:69, A*02:70, A*02:71, A*02:72, A*02:73, A*02:74, A*02:75, A*02:76, A*02:77, A*02:78, A*02:80, A*02:81, A*02:84, A*02:85, A*02:87, A*02:89, A*02:90, A*02:91, A*02:92, A*02:93, A*02:95, A*02:96, A*02:97, A*02:99, A*02:101, A*02:102, A*02:103, A*02:104, A*02:105, A*02:106, A*02:107, A*02:108, A*02:109, A*02:110, A*02:111, A*02:112, A*02:114, A*02:115, A*02:116, A*02:117, A*02:119, A*02:120, A*02:121, A*02:122, A*02:123, A*02:124, A*02:126, A*02:127, A*02:128, A*02:129, A*02:130, A*02:131, A*02:132, A*02:133, A*02:134, A*02:136, A*02:137, A*02:138, A*02:139, A*02:140, A*02:141, A*02:142, A*02:143, A*02:144, A*02:145, A*02:146, A*02:147, A*02:148, A*02:150, A*02:151, A*02:153, A*02:154, A*02:155, A*02:156, A*02:157, A*68:01, A*68:02, A*68:03, A*68:04, A*68:05, A*68:06, A*68:07, A*68:08, A*68:09, A*68:10, A*68:13, A*68:14, A*68:15, A*68:16, A*68:17, A*68:19, A*68:20, A*68:21, A*68:22, A*68:23, A*68:24, A*68:25, A*68:26, A*68:27, A*68:28, A*68:29, A*68:30, A*68:31, A*68:32, A*68:33, A*68:34, A*68:35, A*68:36, A*68:37, A*68:38, A*68:39, A*68:40, A*68:41, A*68:42, A*68:43, A*68:44, A*68:45, A*68:46, A*68:47, A*69:01.

In certain embodiments, the CAR recognizes the 142T144K145H eplet in HLA-A alleles including A*02:01, A*02:02, A*02:03, A*02:04, A*02:05, A*02:06, A*02:07, A*02:08, A*02:09, A*02:10, A*02:11, A*02:12, A*02:13, A*02:14, A*02:16, A*02:17, A*02:18, A*02:20, A*02:21, A*02:22, A*02:24, A*02:25, A*02:26, A*02:27, A*02:28, A*02:29, A*02:30, A*02:31, A*02:33, A*02:34, A*02:35, A*02:36, A*02:37, A*02:38, A*02:40, A*02:41, A*02:42, A*02:45, A*02:46, A*02:47, A*02:48, A*02:49, A*02:50, A*02:51, A*02:52, A*02:54, A*02:55, A*02:56, A*02:57, A*02:58, A*02:59, A*02:60, A*02:61, A*02:62, A*02:63, A*02:64, A*02:66, A*02:67, A*02:68, A*02:69, A*02:70, A*02:71, A*02:72, A*02:73, A*02:74, A*02:75, A*02:76, A*02:77, A*02:78, A*02:80, A*02:81, A*02:84, A*02:85, A*02:87, A*02:89, A*02:90, A*02:91, A*02:92, A*02:93, A*02:95, A*02:96, A*02:97, A*02:99, A*02:101, A*02:102, A*02:103, A*02:104, A*02:105, A*02:106, A*02:107, A*02:108, A*02:109, A*02:110, A*02:111, A*02:112, A*02:114, A*02:115, A*02:116, A*02:117, A*02:119, A*02:120, A*02:121, A*02:122, A*02:123, A*02:124, A*02:126, A*02:127, A*02:128, A*02:129, A*02:130, A*02:131, A*02:132, A*02:133, A*02:134, A*02:136, A*02:137, A*02:138, A*02:139, A*02:140, A*02:141, A*02:142, A*02:143, A*02:144, A*02:145, A*02:146, A*02:147, A*02:148, A*02:150, A*02:151, A*02:153, A*02:154, A*02:155, A*02:156, A*02:157, A*68:01, A*68:02, A*68:03, A*68:04, A*68:05, A*68:06, A*68:07, A*68:08, A*68:09, A*68:10, A*68:13, A*68:14, A*68:15, A*68:16, A*68:17, A*68:19, A*68:20, A*68:21, A*68:22, A*68:23, A*68:24, A*68:25, A*68:26, A*68:27, A*68:28, A*68:29, A*68:30, A*68:31, A*68:32, A*68:33, A*68:34, A*68:35, A*68:36, A*68:37, A*68:38, A*68:39, A*68:40, A*68:41, A*68:42, A*68:43, A*68:44, A*68:45, A*68:46, A*68:47, A*69:01 (Fernandez-Vina et al, in Immunobiology of the Human MHC vol. I, J. A. Hansen (Ed), pp 890-931, 2006; Mulder et al, Human Immunology 56: 106-113, 1997).

In certain embodiments, the CAR is directed specifically against the eplets 142T144K145H and 145KHA in HLA-A alleles including A*02:01, A*02:02, A*02:03, A*02:04, A*02:05, A*02:06, A*02:07, A*02:08, A*02:09, A*02:10, A*02:11, A*02:12, A*02:13, A*02:14, A*02:16, A*02:17, A*02:18, A*02:20, A*02:21, A*02:22, A*02:24, A*02:25, A*02:26, A*02:27, A*02:28, A*02:29, A*02:30, A*02:31, A*02:33, A*02:34, A*02:35, A*02:36, A*02:37, A*02:38, A*02:40, A*02:41, A*02:42, A*02:45, A*02:46, A*02:47, A*02:48, A*02:49, A*02:50, A*02:51, A*02:52, A*02:54, A*02:55, A*02:56, A*02:57, A*02:58, A*02:59, A*02:60, A*02:61, A*02:62, A*02:63, A*02:64, A*02:66, A*02:67, A*02:68, A*02:69, A*02:70, A*02:71, A*02:72, A*02:73, A*02:74, A*02:75, A*02:76, A*02:77, A*02:78, A*02:80, A*02:81, A*02:84, A*02:85, A*02:87, A*02:89, A*02:90, A*02:91, A*02:92, A*02:93, A*02:95, A*02:96, A*02:97, A*02:99, A*02:101, A*02:102, A*02:103, A*02:104, A*02:105, A*02:106, A*02:107, A*02:108, A*02:109, A*02:110, A*02:111, A*02:112, A*02:114, A*02:115, A*02:116, A*02:117, A*02:119, A*02:120, A*02:121, A*02:122, A*02:123, A*02:124, A*02:126, A*02:127, A*02:128, A*02:129, A*02:130, A*02:131, A*02:132, A*02:133, A*02:134, A*02:136, A*02:137, A*02:138, A*02:139, A*02:140, A*02:141, A*02:142, A*02:143, A*02:144, A*02:145, A*02:146, A*02:147, A*02:148, A*02:150, A*02:151, A*02:153, A*02:154, A*02:155, A*02:156, A*02:157, A*68:01, A*68:02, A*68:03, A*68:04, A*68:05, A*68:06, A*68:07, A*68:08, A*68:09, A*68:10, A*68:13, A*68:14, A*68:15, A*68:16, A*68:17, A*68:19, A*68:20, A*68:21, A*68:22, A*68:23, A*68:24, A*68:25, A*68:26, A*68:27, A*68:28, A*68:29, A*68:30, A*68:31, A*68:32, A*68:33, A*68:34, A*68:35, A*68:36, A*68:37, A*68:38, A*68:39, A*68:40, A*68:41, A*68:42, A*68:43, A*68:44, A*68:45, A*68:46, A*68:47, A*69:01 (Fernandez-Vina et al, in Immunobiology of the Human MHC vol. I, J. A. Hansen (Ed), pp 890-931, 2006; Mulder et al, Human Immunology 56: 106-113, 1997).

In some embodiments, the CAR is not directed against the eplets 44RME, 105S, and 127K, as described in the Examples herein.

In some embodiments, the CAR does not recognize HLA that do not contain the 144TKH eplet.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The antibody scaffold can include naturally-occurring amino acid sequences or can be engineered, designed, or modified so as to provide desired and/or improved properties, e.g., binding affinity. Generally, the binding affinity of an antigen-binding domain, e.g., an antibody, for a target antigen (e.g., CD19 antigen) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen is an antigen-binding domain that binds the antigen with high affinity and does not significantly bind other unrelated antigens.

In some embodiments, the antibody scaffold is a humanized antibody or fragments thereof. A “humanized” antibody is an antibody in which all or substantially all CDR amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody, refers to a variant of the non-human antibody that has undergone humanization, in some cases to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the heavy and light chains of an antibody can be full-length or can be an antigen-binding portion (a Fab, F(ab′)₂, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that includes a portion of an intact antibody that binds the antigen to which the intact antibody binds. Examples of antibody fragments include, but are not limited to, Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments including a variable heavy chain region and/or a variable light chain region, such as scFvs.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain including four conserved framework regions (FRs) and three CDRs. (See, e.g., Kindt et al. “Kuby Immunology”, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J Immunol (1993) 150:880-87; Clarkson et al., Nature (1991) 352:624-28.

Single-domain antibodies are antibody fragments including all or a portion of the heavy chain variable domain or all or a portion of the light chain variable domain of an antibody. In some embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments including arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the CAR's antibody scaffold (i.e., extracellular antigen-binding domain) includes (e.g., is composed of) a single chain variable fragment (scFv) derived from a fusion protein of the variable regions of the heavy and light chains of an antibody. In some embodiments, also useful are scFvs derived from Fab fragments (instead of from an antibody, e.g., obtained from Fab libraries). In some embodiments, the CAR of the disclosure includes a scFv fused to the TMD and then to the intracellular domain. “First-generation” CARs include those that solely provide CD3-chain induced signal upon antigen binding. “Second-generation” CARs include those that provide both CD3-chain induced signal upon antigen binding and co-stimulation, such as one including an intracellular signaling domain from a costimulatory receptor (e.g., CD28 or 4-1BB). “Third-generation” CARs include those that include multiple co-stimulatory domains of different costimulatory receptors. A fourth generation of CAR-T cell includes CAR-T cells redirected for cytokine killing (TRUCK), where the vector containing the CAR construct includes a cytokine cassette.

The antigen binding domain of the CAR of the disclosure includes the CDRs of a human anti-HLA-A2 antibody grafted on to an antibody scaffold.

In some embodiments, the antibody scaffold is a single chain antibody. Preferably, the antibody is a humanized antibody. Particularly, such antigen binding domain is an antibody fragment selected from fragment antigen binding (Fab) fragments, F(ab′)2 fragments, Fab′ fragments, Fv fragments, recombinant IgG (rIgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antibodies are single-chain antibody fragments comprising a variable heavy chain region and/or a variable light chain region, such as scFv.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that may not be produced by enzyme digestion of a naturally-occurring intact antibody. In some aspects, the antibody fragments are scFvs.

In embodiments wherein the antibody scaffold is a scFv, the scFv can be derived from the variable heavy chain (VH) and variable light chain (VL) regions of an antigen-specific mAb linked by a flexible linker. The scFv retains the same specificity and a similar affinity as the full antibody from which it was derived (Muniappan et al. (2000) Cancer Gene Ther. 7: 128-134). Various methods for preparing an scFv can be used including methods described in U.S. Pat. No. 4,694,778; Bird et al. (1988) Science 242:423-442; Ward et al. (1989) Nature 334:54454; and Skerra et al. (1988) Science 242: 1038-1041. In certain embodiments, the scFv may be a humanized or is a fully human scFv.

In some embodiments, the antibody scaffold is an anti-HLA-A2 scFv produced from human B-cell derived hybridoma clone SN607D8. This hybridima produces an IgG1κ monoclonal antibody that recognizes HLA serotypes A2 and A28 as described in Mulder et al., Identification, isolation, and culture of HLA-A2-specific B lymphocytes using MHC class I tetramers. J Immunol. (2003) 171:6599-603.

In some embodiments, the antibody scaffold contains the CDRs of anti-HLA-A2 as follows:

| Complementarity | | SEQ ID |
|---|---|---|
| Determining Region (CDR) | Sequence | NO |
| CDR H1 | YTFTDYYIH | 1 |
| CDR H2 | WMAWISPHTGGTIY | 2 |
| CDR H3 | RGPDDWNDGDAFDI | 3 |
| CDR L1 | QSVLYSSNNENFLA | 4 |
| CDR L2 | LLIYWASTRES | 5 |
| CDR L3 | QQYYSTPI | 6 |

In some embodiments, the antibody scaffold is human trastuzumab.

In one embodiment, the heavy chain of trastuzumab contains the amino acid sequence of SEQ ID NO: 7 below:

EVQLVESGGGLVQPGGSLRLSCAASGFNIKDTYIHWVRQAAPGKGLEWV
ARIYPTNGYTRYADSVKGRFTISADTSKNTAYLQMNSLRAEDTAVYYCS
RWGGDGFYAMDYWGQGTL

In one embodiment, the light chain of trastuzumab contains the amino acid sequence of SEQ ID NO: 8 below:

DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEI

As described supra, antigen targeting or antigen recognition by CAR molecules most commonly involves the use of a single chain variable fragment (scFv) that has been assembled from a monoclonal antibody. However, alternative targeting moieties include ligands (Altenschmidt et al. (1996) Clin. Cancer Res. 2: 1001-8; Muniappan, et al. (2000) Cancer Gene Ther. 7: 128-134), peptides (Pameijer et al. (2007) Cancer Gene Ther. 14:91-97), chimeric ligands (Davies et al. (2012) Mol. Med. 18:565-576), receptor derivatives (Zhang et al. (2012) J Immunol. 189:2290-9), and single domain antibodies (Sharifzadeh et al. (2012) Cancer Res. 72: 1844-52). Any desired antibody or antibody fragment thereof that specifically recognizes and binds a target antigen, in particular HLA-A2 isoforms as defined above, may be incorporated in a CAR according to the disclosure.

In some aspects, the antibody scaffold may be derived from an antibody or fragment thereof that has one or more specified functional features, such as binding properties, including binding to particular epitopes, such as epitopes that are similar to or overlap with those of other antibodies, the ability to compete for binding with other antibodies, and/or particular binding affinities. In some embodiments, the antigen binding domain, the CARs comprising such, and the cells comprising such CARs display a binding preference for target antigen-expressing cells as compared to target antigen-negative cells. In some embodiments, the binding preference is observed where a significantly greater degree of binding is measured to the antigen-expressing, as compared to the non-expressing cells. In some cases, the total degree of observed binding to the target antigen or to the antigen-expressing cells is approximately the same, at least as great or greater than that observed for non-antigen specific domains, CARs, or cells. In any of the provided embodiments, comparison of binding properties, such as affinities or competition, may be via measurement by assays known in the art such as mentioned above.

The CAR of the disclosure also includes a hinge domain, a transmembrane domain, and an intracellular domain.

The hinge domain refers to a flexible polypeptide connector region providing structural flexibility and spacing to flanking polypeptide regions. The hinge can consist of natural or synthetic polypeptides.

The transmembrane domain can include a sequence derived from IgG subclass IgG1, IgG4, IgD or CD8. In certain embodiments, the transmembrane domain is a CD28 or a CD8 transmembrane domain. The transmembrane domain can have any length. In some embodiments, the transmembrane domain includes 1 amino acid or 10 amino acids or 20 amino acids or 50 amino acids or 60 amino acids or 70 amino acids or 80 amino acids or 100 amino acids or 120 amino acids or 140 amino acids or 160 amino acids or 180 amino acids or 200 amino acids or 250 amino acids or 300 amino acids or any number therebetween.

In some embodiments, the intracellular domain includes a primary signaling domain, optionally derived from a CD3 chain domain. The CD3 chain can be selected from the group consisting of: a CD3 zeta (CD3ζ) chain, a CD3 gamma (CD3γ) chain, a CD3 delta (CD3δ) chain, a CD3 epsilon (CD3ε) chain, derivatives, mutants, variants, fragments and combinations thereof. In certain embodiments, the primary signaling domain optionally further includes an Fc domain from the immunoglobulin superfamily, such as for example, FcγRI (CD64), FcγRIIA (CD32), FcγRIIB (CD32), FcγRIIIA (CD16a), FcRIII1B (CD16b), FcaRI (CD89), FcRI, FcRII (CD23), Fca, Fc R, derivatives, mutants, variants, fragments and combinations thereof. In some embodiments, the Fc domain is an Fc7 domain, derivatives, mutants, variants, fragments and combinations thereof. As used herein, an “Fc7 domain” includes FcγRI (CD64), FcγRIIA (CD32), FcγRIIB (CD32), FcγRIIIA (CD16a), FcγRIIIB (CD16b) derivatives, mutants, variants, and fragments thereof.

The intracellular domain can also comprise a costimulatory domain of a protein selected from CD28, ICOS, CTLA4, 41BB, CD27, CD30, CD132, OX-40, TACI, GITR, HVEM, TIM3, other TNFR superfamily members, and derivatives, mutants, variants, fragments and combinations thereof.

In certain embodiments, an intracellular domain can be CD28.

In some embodiments, the intracellular domain includes both a costimulatory domain and a CD3 domain, In one embodiment, the costimulatory domain is derived from CD28 and the CD3 domain is derived from CD3ζ.

In certain embodiments, the intracellular domain does not comprise a signaling domain.

In some embodiments, a CAR can further comprise a linker region. The linker may be rich in glycine, serine, and/or threonine for solubility. The linker region can connect to N-terminus of variable heavy (VH) chain with the C-terminus of the variable light (VL) chain or vice versa.

### Regulatory T Cells (Trees)

Tregs are important in the maintenance of immune cell homeostasis as evidenced by undesirable consequences of genetic or physical ablation of the Treg population. Treg cells generally maintain order in the immune system by enforcing a dominant negative regulation on other immune cells. Broadly classified into natural or adaptive (induced) Tregs; natural Tregs are CD4⁺CD25⁺ T-cells which develop, and emigrate from the thymus to play a role in immune homeostasis. Adaptive Tregs are non-regulatory CD4⁺ T-cells which acquire CD25 (IL-2R alpha) expression outside of the thymus, and may be induced by inflammation and disease processes, such as autoimmunity and cancer.

There is increasing evidence that Tregs acquire their function through a myriad of mechanisms that may include the secretion of immunosuppressive soluble factors such as IL-9, IL-10 and TGF beta, cell contact mediated regulation via the high affinity TCR and other costimulatory molecules such as CTLA-4, GITR, and cytolytic activity. Under the influence of TGF beta, adaptive Treg cells mature in peripheral sites, including mucosa-associated lymphoid tissue (MALT), from CD4⁺ Treg precursors, where they acquire the expression of markers typical of Tregs, including CD25, CTLA4 and GITR/AITR. Upon up-regulation of the transcription factor Foxp3, Treg cells begin their suppressive effect. This includes the secretion of cytokines including IL-10 and TGF beta which may induce cell-cycle arrest or apoptosis in effector T cells, and blocking co-stimulation and maturation of dendritic cells.

In general, T regulatory cells have been identified as a CD4⁺CD25⁺ T cell population capable of suppressing an immune response. The identification of Foxp3 as a “master-regulator” of Tregs helped define Tregs as a distinct T cell lineage. The identification of additional antigenic markers on the surface of Tregs has enabled identification and FACS sorting of viable Tregs to greater purity, resulting in a more highly-enriched and suppressive Treg population. For example, the transcription factor, HELIOS, is expressed in a large subset of Foxp3+ Tregs (Thornton et al., Helios+ and Helios− Treg subpopulations are phenotypically and functioanlly distinct and express dissimilar TCR repertoires, Eur. J. Immunol. 2019 49(3):398-412). In addition to CD4 and CD25, both mouse and human Tregs express GITR/AITR, CTLA-4, and express low levels of CD127 (IL-7Ra). Moreover, Tregs can exist in different states which can be identified based on their expression of surface markers. Tregs which develop in the thymus from CD4⁺ thymocytes are known as “natural” Tregs, however Tregs can also be induced in the periphery from naïve CD4⁺ T cells in response to low-dose engagement of the TCR, TGF beta and IL-2. These “induced” Tregs secrete the immunosuppressive cytokine IL-10. The phenotype of Tregs changes again as they become activated, and markers including GARP in mouse and human, CD45RA in human, and CD103 in mouse have been shown to be useful for the identification of activated Tregs.

Accordingly, in some embodiments, an isolated regulatory T cell (Treg) is modified to express a chimeric antigen receptor (CAR) comprising complementarity determining regions (CDRs) of an anti-HLA-A2 antibody grafted onto an antibody scaffold, a hinge domain, a transmembrane domain, and an intracellular domain. The intracellular domain can comprise a primary signaling domain, optionally derived from a CD3 chain domain, and a second signaling domain which is a costimulatory domain such as CD28.

In certain embodiments, the primary signaling domain is or comprises a CD3 chain domain, wherein the CD3 chain is selected from the group consisting of: a CD3 zeta (CD3ζ) chain, a CD3 gamma (CD3γ) chain, a CD3 delta (CD3δ) chain, a CD3 epsilon (CD3ε) chain, derivatives, mutants, variants, fragments and combinations thereof.

In certain embodiments, the intracellular domain comprises only a CD3 domain.

In certain embodiments, the intracellular domain comprises only a costimulatory domain.

In some embodiments, the intracellular domain does not include a signaling domain.

In certain embodiments, a Treg costimulatory signaling domain comprises CD28.

In certain embodiments, the Treg cell is CD25⁺, FOXP3⁺ HELIOS⁺, and contains a demethylated Treg-specific demethylated region (TSDR).

In certain embodiments, the CAR-Treg suppresses in vitro proliferation of T cells in the presence of HLA-A2+ cells. Methods of measuring in vitro proliferation of T cells are well known in the art.

In certain embodiments, the CAR-Treg accumulates in HLA-A2 expressing tissue in vivo.

In certain embodiments, the CAR-Treg suppresses the immune response in the presence of HLA-A2+ and HLA-A28+ cells in vivo.

### Nucleic Acids

In another aspect, some embodiments of the disclosure relate to exogenous nucleic acid molecules including nucleotide sequences encoding the chimeric antigen receptors of the disclosure, including expression cassettes, and expression vectors containing these nucleic acid molecules operably linked to heterologous nucleic acid sequences such as, for example, regulatory sequences which allow in vivo expression of the receptor in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric antigen receptor (CAR) including, from N-terminus to C-terminus: a) an antigen-binding domain comprising the complementarity determining regions (CDRs) of an anti-HLA-A2 antibody grafted onto an antibody scaffold; b) a hinge domain; c) a transmembrane domain; and d) an intracellular domain.

Nucleic acids of the disclosure can further include one or more polynucleotides of interest. The polynucleotide of interest can be a regulatory or signaling nucleic acid, or can encode any protein that can be expressed by the engineered cell. In some embodiments, the protein is a detectable label. In some embodiments, the detectable label is a fluorescent protein or a chromogenic protein. Suitable fluorescent proteins include, without limitation, GFP, mCherry, mTomato, mStrawberry, and others.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric receptor as disclosed herein, which is operably linked to expression control elements, such as a promoter, and optionally, any or a combination of other nucleic acid sequences that affect the transcription or translation of the coding sequence.

Additional promoter elements, e.g., enhancers, can regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the exogenous nucleic acid encoding the CAR is codon-optimized. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell. In some embodiments, the exogenous nucleic acid is codon optimized for expression in a mammalian cell. In some embodiments, the exogenous nucleic acid is codon optimized for expression in a human cell.

As described supra, in some embodiments, the disclosure provides one or more vectors for use in modifying a Treg into a chimeric-antigen-receptor regulatory T cell (CAR-Treg cell). In some embodiments, the CAR-Treg is for therapeutic use in a transplant patient.

In some embodiments, the or more vectors contain an exogenous nucleic acid encoding a chimeric antigen receptor having complementarity determining regions (CDRs) of an anti-HLA-A2 antibody grafted onto an antibody scaffold, a hinge domain, a transmembrane domain, and an intracellular domain as described herein.

In some embodiments, the one or more vectors contain an exogenous nucleic acid encoding a chimeric antigen receptor having the amino acid sequence of SEQ ID NO: 12 as follows:

MALPVTALLLPLALLLHAARPEQKLISEEDLEISEVQLVESGGGLVQPG
GSLRLSCAASGYTFTDYYIHWVRQAPGKGLEWMAWISPHTGGTIYADSV
KGRFTISADTSKNTAYLQMNSLRAEDTAVYYCARGPDDWNDGDAFDIWG
QGTLVTVSSGGGGSGGGGSGGGGSSDIQMTQSPSSLSASVGDRVTITCK
SSQSVLYSSNNENFLAWYQQKPGKAPKLLIYWASTRESGVPSRFSGSRS
GTDFTLTISSLQPEDFATYYCQQYYSTPITFGQGTKVEIKESKYGPPCP
PCPMFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIG
MKGERRRGKGHDGLYQGLSTATKDTYDALHMQALPPR

It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

### Engineered Regulatory T Cells

Exemplary methods of isolating cells and engineering these cells with a CAR are described in the Examples section which follows.

In some embodiments, preparation of the engineered cells includes one or more culture and/or preparation steps. The cells for introduction of the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In certain embodiments, a biological sample is obtained from one or more sources comprising: autologous, allogeneic, haplotype matched, haplotype mismatched, haplo-identical, xenogeneic, cell lines or combinations thereof.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be useful where no antibody is available that specifically identifies a cell type in a heterogeneous population, such that separation is carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing one or more markers, e.g., CD4⁺CD25⁺, FOXP3⁺ and Helios⁺.

T cells, are isolated by positive or negative selection techniques. For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker 1″) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD 14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some aspects, a CD4 expression-based selection step is used to generate the CD4⁺ cell population or sub-population, such that both the positive and negative fractions from the CD4-based separation are retained and used in subsequent steps of the methods, optionally following one or more further positive or negative selection steps.

In one example, a sample of PBMCs or other white blood cell sample is subjected to selection of CD4⁺ cells, where both the negative and positive fractions are retained. The negative fraction then is subjected to negative selection based on expression of, for example, CD 14 and CD45RA, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naïve CD4⁺ T lymphocytes are CD45RO⁺, CD45RA⁺, CD62L⁺, or CD4⁺ T cells. In some embodiments, central memory CD4⁺ cells are CD62L⁺ and CD45RO⁺.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 edited by: S. A. Brooks and U. Schumacher © Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen, U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto are capable of being attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9):651-60, Terakura et al. (2012) Blood 1:72-82, and Wang et al. (2012) Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-73; and Godin et al. (2008) J Biophoton. 1(5):355-76. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al.; Klebanoff et al. (2012) J Immunother. 35(9): 651-60, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25° C., generally at least about 30° C., and generally at or about 37° C. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

The nucleic acids of the disclosure can be introduced into a host cell, such as, for example, a regulatory T cell, to produce a recombinant engineered cell containing the nucleic acid molecule. Accordingly, some embodiments of the disclosure relate to methods for making recombinant cells, including the steps of: (a) providing a cell capable of protein expression and (b) contacting the provided cell with at least one of the recombinant nucleic acids described herein.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In some embodiments, the nucleic acid molecules are delivered by viral or non-viral delivery vehicles known in the art. For example, the nucleic acid molecule can be stably integrated in the host genome, or can be episomally replicating, or present in the recombinant host cell as a mini-circle expression vector for transient expression. In some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques, or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In order to assess the expression of a CAR polypeptide of the disclosure, or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al, 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the exogenous nucleic acid of the Treg is integrated into the TCR alpha constant (TRAC) locus of the CAR-Treg. An exemplary method is described in the Examples herein. This can also be performed using methods known in the art such as those described in U.S. Patent Application Publication No. US2019/0119638 and Eyquem et al., “Targeting a CAR to the TRAC Locus with CRISPR/Cas9 Enhances Tumour Rejection,” Nature 2017 543(7643):113-117. A previous study has shown that site-specific integration of a CD19-CAR into the TCR alpha constant region (TRAC) of T cells results in a more uniform distribution and TCR-like regulation of CAR surface expression, thereby mitigating T-cell exhaustion and enhancing anti-tumor activity (13).

Briefly, to generate the CAR-Treg described herein, the exogenous nucleic acid can be cloned into a targeting construct, which provides for targeted integration of the transgene at a site within the genome. In a preferred embodiment, a nucleic acid encoding a CAR is cloned into a targeting construct, which provides for targeted integration of the nucleic acid sequence encoding the CAR at a site within the genome, in a particular embodiment, a site that disrupts expression of a gene encoding a protein required for expression of a functional TCR complex in the cell. For example, a transgene, for example, a polynucleotide encoding a CAR, of the invention can be cloned into a suitable targeting construct, or a suitable vector such as a retroviral vector, and introduced into the T cell using well known molecular biology techniques (see Ausubel et al., Current Protocols in Molecular Biology, John Wiley and Sons, Baltimore, Md. (1999)).

Any suitable targeting construct suitable for expression in a cell of the present disclosure, particularly a human Treg, can be employed. In a particular embodiment, the targeting construct is compatible for use with a homologous recombination system suitable for targeted integration of the nucleic acid sequence (transgene) at a site within the genome of the cell. Exemplary homologous recombination systems are well known in the art and include, but are not limited to, technologies utilizing a nuclease, for example, transcription activator-like effector nucleases (TALENs), Zinc-finger nucleases (ZFNs), clustered regularly interspaced short palindromic repeats (CRISPRs) systems such as and CRISPR associated protein 9 (Cas9) and Cpf1, and/or Meganuclease or a Mega-Tal (fusion of a Tal domain and a Meganuclease) and the like, which provide for homologous recombination, for example, a desired target site within the genome of the cell (see Examples; see also U.S. Pat. No. 8,697,359; US publication 20140068797; Gaj et al., Trends Biotechnol. 31:397-405 (2013); Gersbach et al., Nucl. Acids Res. 39:7868-7878 (2011); Vasileva, et al. Cell Death Dis. 6:e1831. (Jul. 23, 2015); Sontheimer, Hum. Gene Ther. 26(7):413-424 (2015); Osborn et al., Mol. Ther. 24(3):570-581 (2016))). Such methods are well known in the art and commercially available (ThermoFisher, Carlsbad, Calif; GenScript, Piscataway, N.J.; Clontech, Mountain View, Calif.). Other CRISPR based systems include pyrogen and Aureus. Such methods can be used to carry out or promote homologous recombination.

As discussed above, some embodiments of the disclosure relate to methods for making a recombinant cell, including (a) providing a cell capable of protein expression and (b) contacting the provided cell with a recombinant nucleic acid of the disclosure. In some embodiments, the method includes providing a plurality of cells capable of protein expression and (b) contacting the plurality of cells with a plurality of recombinant nucleic acids of the disclosure,

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the chimeric receptors, nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., a carrier.

Accordingly, one aspect of the present disclosure relates to pharmaceutical compositions that include a pharmaceutically acceptable carrier and one or more of the following: (a) a chimeric antigen receptor of the disclosure; (b) a recombinant nucleic acid of the disclosure; and/or (c) a recombinant cell of the disclosure. In some embodiments, the composition includes (a) a recombinant nucleic acid of the disclosure and (b) a pharmaceutically acceptable carrier. In some embodiments, the composition is formulated for introducing the recombinant nucleic acid into a cell. In some embodiments, the recombinant nucleic acid is encapsulated in a lipid nanoparticle (LNP), liposome, or viral particle. In some embodiments, the composition includes (a) a recombinant cell of the disclosure and (b) a pharmaceutically acceptable carrier.

In certain embodiments, the pharmaceutical compositions in accordance with some embodiments disclosed herein include cell cultures that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying which yields a powder of the active ingredient plus any additional desired ingredient from a previously sterile-filtered solution thereof.

### III. Methods

As described supra, solid organ transplantation remains an important therapy option for patients with end-stage organ dysfunction. However, treatment necessitates life-long immunosuppression to prevent allograft rejection. Unfortunately, immunosuppressive regimens can be directly toxic to both host and transplanted organs, and also increase patient susceptibilities to cancer development and infections. Thus, strategies to reduce the burden of immunosuppression would reduce the side effects of the current medication. One mechanism is the induction of immune tolerance. As such, the present disclosure provides a method for inducing immune tolerance for organ transplantation that involves providing a population of CAR-Tregs of the present disclosure and administering the population of CAR-Tregs to a subject having received an organ transplant. Administration of any one of the therapeutic compositions described herein, e.g., chimeric antigen receptors, nucleic acids, recombinant cells, and pharmaceutical compositions, can be used to treat subjects, e.g., organ transplant patients. In some embodiments, the chimeric receptors, nucleic acids, recombinant cells, and pharmaceutical compositions described herein can be incorporated into therapeutic agents for use in methods of treating an individual who has, who is suspected of having, or who may be at high risk for developing one or more transplant-related health conditions, such as transplant rejection and graft versus host disease.

Immune tolerance can involve rendering the immune system unable to reject the transplant. Tolerance can be measured in various ways including, but not limited to, loss of cytotoxic T cells that recognize transplant antigens, unresponsiveness of T cells to transplant antigen, and lack of anti-donor antibody production. In addition to unresponsiveness, tolerance can also manifest as deviation of the donor-reactive T cells to less pathogenic the more tolerogenic type, e.g. reduction in Th1 cytokine production, increase in Th2 cytokines, IL-10, and TGFb, increase of donor reactive Tregs.

The CAR-T cells, once they have been expanded ex vivo, can be reinfused into the subject in a therapeutically effective amount. The term “therapeutically effective amount” as used herein means the amount of CAR T cells when administered to a mammal, in particular a human, in need of such treatment, is sufficient to induce immune tolerance and/or treat organ rejection etc.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric patients. As T cells replicate and expand after transfer, the administered cell dose may not resemble the final steady-state number of cells.

For use in the various embodiments described herein, an effective amount or number of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, at least 1×10⁷ cells, at least 2×10⁷ cells, at least 3×10⁷ cells, at least 4×10⁷ cells, at least 5×10⁷ cells, at least 6×10⁷ cells, at least 7×10⁷ cells, at least 8×10⁷ cells, at least 9×10⁷ cells, at least 1×10⁸ cells, at least 2×10⁸ cells, at least 3×10⁸ cells, at least 4×10⁸ cells, at least 5×10⁸ cells, at least 6×10⁸ cells, at least 7×10⁸ cells, at least 8×10⁸ cells, at least 9×10⁸ cells, at least 1×10⁹ cells, at least 2×10⁹ cells, at least 3×10⁹ cells, at least 4×10⁹ cells, at least 5×10⁹ cells, at least 6×10⁹ cells, at least 7×10⁹ cells, at least 8×10⁹ cells, at least 9×10⁹ cells, or multiples thereof. The recombinant cells can be derived from one or more donors, or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. In some embodiments, a sign or symptom or marker of a disease is ameliorated when the treatment is found to decrease, suppress, attenuate, diminish, arrest, or stabilize the development or progression of a disease or symptom or marker. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with transplant rejection.

Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. An exemplary method is described in the Examples section which follows.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, Cas12a (Cpf1), Cas13a (C2c2), Cas13b, Cas13d, CasF, CasG, CasH, Csy1, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

In some embodiments, a recombinant adeno-associated virus (rAAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

The CAR-T cells, once they have been expanded ex vivo can be reinfused into the subject in a therapeutically effective amount.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the CAR T cells of the present disclosure may be administered at a dosage of 10⁴ to 10¹⁰ total cells. In another embodiment, a pharmaceutical composition including the CAR T cells of the present disclosure may be administered at a dosage of 10³ to 10⁸ cells/kg body weight, including all integer values within those ranges.

Compositions including the CAR T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

In some embodiments, administration of any of the compositions embodied herein, for the treatment of, for example, transplant rejection, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, including a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR-T cells. In general, the compositions are prepared by uniformly and intimately bringing into association any active ingredients with liquid carriers.

In some embodiments, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The invention also contemplates the combination of the composition of the present invention with other drugs and/or in addition to other treatment regimens or modalities such as immunosuppressive drugs. In some embodiments, administration of the CAR-Tregs of the present disclosure induces immune tolerance to transplanted cells and tissue such that a dose of immunosuppressive drug can be reduced or eliminated.

In certain embodiments, the organ transplant is a non-vascularized transplant. Exemplary non-vascularized transplants include, without limitation, skin and pancreatic islets.

In some embodiments, the organ transplant is a vascularized transplant. Exemplary vascularized transplants include, without limitation, heart, kidney, liver, pancreas, small intestine, or a composite tissue allograft.

### IV. Systems and Kits

Also provided herein are systems and kits including the chimeric antigen receptors, recombinant nucleic acids, recombinant cells, or pharmaceutical compositions provided and described herein as well as written instructions for making and using the same. For example, provided herein, in some embodiments, are systems and/or kits that include one or more of the following: a chimeric receptor as described herein, a recombinant nucleic acid as described herein, a recombinant cell as described herein, or a pharmaceutical composition as described herein.

In some embodiments, the systems and/or kits of the disclosure further include one or more syringes (including pre-filled syringes) and/or catheters (including pre-filled syringes) used to administer one any of the provided chimeric receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

Also provided herein are systems and kits including the chimeric receptors, recombinant nucleic acids, recombinant cells, or pharmaceutical compositions provided and described herein as well as written instructions for making and using the same. For example, provided herein, in some embodiments, are systems and/or kits that include one or more of the following: a chimeric receptor as described herein, a recombinant nucleic acid as described herein, a recombinant cell as described herein, or a pharmaceutical composition as described herein.

In some embodiments, the systems and/or kits of the disclosure further include one or more syringes (including pre-filled syringes) and/or catheters (including pre-filled syringes) used to administer one any of the provided chimeric receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

Although the present disclosure has been described in some detail by way of illustration and example for purposes of clarity and understanding, it will be apparent that certain changes and modifications can be practiced within the scope of the appended claims.

### EXAMPLES

Transplantation restores the function of diseased organs and cells, but transplant recipients must commit to lifelong immunosuppressive treatments that blunt protective immunity. Research in the past two decades has revealed that regulatory T cells (Tregs) restrain immune activities to avoid the destruction of normal tissue and cells. When directed to the transplanted tissue, Tregs can also protect them from immune-mediated rejection. Presented herein, are results from human Tregs redirected to transplant antigen HLA-A2 using a chimeric antigen receptor (CAR), while deleting the Treg's endogenous T cell receptor (TCR), using CRISPR/Cas9 genome editing to create Tregs with a single specificity to HLA-A2 on transplanted tissue. These engineered mono-specific anti-HLA-A2 CAR Tregs suppressed conventional T cell proliferation in vitro and trafficked to HLA-A2-expressing pancreatic islets transplanted in immunodeficient mice. Moreover, anti-HLA-A2 CAR Tregs prevented graft-vs-host disease, a devastating disease that arises when transplanted immune cells attack the recipient, in humanized mice. Altogether, it is demonstrated that it is feasible to engineer human Tregs with exquisite precision and specificity to direct their potent immunosuppressive function locally to the transplanted tissue.

### Materials and Methods

Human peripheral blood products and T cell isolation and expansion: Human peripheral blood from de-identified healthy donors was purchased from STEMCELL Technologies (Vancouver, Canada), which collects and distributes de-identified human blood products with consent forms, according to protocols approved by the Institutional Review Board (IRB). Peripheral blood mononuclear cells (PBMCs) were isolated by Ficoll (GE Healthcare, Chicago, IL) density gradient centrifugation. T cells were further enriched using the EasySep Human T Cell Isolation Kit (STEMCELL Technologies), as per the manufacturer's instructions. Enriched CD3⁺ T cells, or CD4⁺CD127⁺CD25^{low} conventional T cells (Tconv) or CD4⁺CD127^{low}CD25^{high} regulatory T cells (Tregs) purified by fluorescence-assisted cell sorting (FACS) using a BD FACS Aria II Cell Sorter (Beckton Dickinson, Franklin Lakes, NJ) were used for experiments. Tregs were expanded as previously described (15). Antibodies utilized for flow cytometry are summarized in Table 1 below.

TABLE 1
Flow cytometry antibodies used in the study.

| Antigen | Fluorophore | Reactivity | Clone | Company | Location |
|---|---|---|---|---|---|
| MYC-tag | A647 | Human | 9B11 | Cell Signaling Technologies | Danvers, |
| | | | | | MA |
| CD69 | PE/Cy7 | Human | FN50 | Biolegend | San |
| | | | | | Diego, |
| | | | | | CA |
| CD71 | FITC | Human | CY1G4 | Biolegend | San |
| | | | | | Diego, |
| | | | | | CA |
| CD4 | PE/Cy7 | Human | SK3 | Biolegend | San |
| | | | | | Diego, |
| | | | | | CA |
| ICOS | BV711 | Human | DX29 | BD Biosciences | San Jose, |
| | | | | | CA |
| FOXP3 | e450 | Human | PCH101 | eBioscience | San |
| | | | | | Diego, |
| | | | | | CA |
| CTLA4 | PerCP-e710 | Human | 14D3 | eBioscience | San |
| | | | | | Diego, |
| | | | | | CA |
| EGFR | PE | Human | AY13 | Biolegend | San |
| | | | | | Diego, |
| | | | | | CA |
| CD4 | PE | Human | SK3 | Biolegend | San |
| | | | | | Diego, |
| | | | | | CA |
| CD8 | PerCP | Human | SK1 | Biolegend | San |
| | | | | | Diego, |
| | | | | | CA |
| CD45 | PE/Cy7 | Mouse | 30-F11 | Biolegend | San |
| | | | | | Diego, |
| | | | | | CA |
| HLA-A2 | APC | Human | BB7.2 | Biolegend | San |
| | | | | | Diego, |
| | | | | | CA |
| HELIOS | FITC | Human | 22F6 | Biolegend | San |
| | | | | | Diego, |
| | | | | | CA |
| CD45 | e450 | Human | HI30 | eBioscience | San |
| | | | | | Diego, |
| | | | | | CA |
| Ghost | BV510 | | Viability dye | Tonbo Biosciences | San |
| | | | | | Diego, |
| | | | | | CA |

Cloning and specificity verification of an anti-HLA-A2 scFv: A human B-cell derived hybridoma (clone SN607D8) was used as source material to produce an anti-HLA-A2 scFv. This hybridoma produces an IgG1κ monoclonal antibody that recognizes HLA serotypes A2 and A28 (16). RNA from the SN607D8 hybridoma was used as template for RT-PCR amplification of the VL and VH chains of the IgG. The scFv gene was then constructed in a VH-(GGGS)₃linker-VL format and incorporated into the pHEN1 phage display vector (17). The binding activity of phage-displayed scFv was assessed using two tumor cell lines, THP-1 (HLA-A*02:01/02:01, HLA-B*15:11/15:11 (18)) and RPMI 8226 (HLA-A*30:01/68:02, HLA-B*15:03/15:10 (19)). Binding to these cell lines was measured using sequential staining with a biotinylated anti-phage antibody and fluorochrome-conjugated streptavidin followed by flow cytometric analysis.

Grafting of the anti-HLA-A2 scFv: The CDR regions of the anti-HLA-A2 scFv from hybridoma SN607D8 were grafted onto the 4D5 human antibody scaffold used in herceptin (trastuzumab) by pairwise alignment of amino acid residues using the software Jalview (20). The specific CDR3 regions of the anti-HLA-A2 scFv were predicted using the software Paratome (21). The grafted scFv was constructed in the VH-(GGGGS)₃linker-VL format.

Lentivirus production: The A2-specific CAR was created by generating a chimeric DNA sequence encoding a MYC-tag upstream of the grafted anti-HLA-A2 scFv, an IgG4 hinge, CD28 transmembrane domain, and a CD28− CD3zeta tandem signaling domain (purchased as gblocks from Integrated DNA Technologies, IDT, Coralville, IA). The resulting DNA fragment was subcloned into a pCDH lentiviral vector containing an EF 1 a promoter (addgene-plasmid-64874 (22)). The CAR construct was linked to a truncated EGFR (EGFRt) or a luciferase gene via a 2A self-cleaving peptide sequence. All constructs used in subsequent experiments were confirmed by Sanger sequencing. Lentivirus was produced as previously described (23). Briefly, HEK293T cells were seeded at 3×10⁶ cells on 10 cm cell culture dishes 24 hours prior to transfection with 4 μg of plasmid DNA, 2 μg of the packaging vector pCMV-dR8.9, 2 μg of VSV envelope vector pMD2.G and 15 nmol linear 25 kDa polyethylenimine (Millipore Sigma, Burlington, MA). Media was replaced 24 hours later and ViralBoost Reagent (Alstem, Richmond, CA) was added. The supernatant was collected 24 and 48 hours later. Virus was concentrated using LentiX concentrator (Takara, Shiga, Japan).

AAV6 production: A pAAV-MCS plasmid containing inverted terminal repeats (ITRs) from AAV serotype 2 (Agilent Technologies, Santa Clara, CA) was utilized as backbone for AAV6 plasmid construction (naturally occurring AAV6 has an AAV2 ITR (24)). Cloning was performed with in-fusion cloning tools and protocols provided by Takara. Large scale DNA preparation was performed using a Zymopure plasmid maxiprep kit (Zymo Research, Irvine, CA). All constructs used in subsequent experiments were confirmed by Sanger sequencing. For AAV production, 30 μg of pDGM6 helper plasmid (a gift from Dr. YY Chen, University of California, Los Angeles), 40 μg of pAAV helper, and 15 nmol linear polyethylenimine were used. AAV6 vector production was carried out by iodixanol gradient purification (25). After ultracentrifugation, AAVs were extracted by puncture and further concentrated using a 50 ml Amicon column (Millipore Sigma) and titrated directly on primary human T cells.

HLA allele cross-reactivity assay: HLA allele cross-reactivity of the A2-CAR-expressing Tregs was determined based on a previously reported method (26). In brief, 2.5×10⁴ FACS-purified A2-CAR Tregs, as well as 2.5×10⁴ control untransduced polyclonal Tregs, were incubated with 0.5 μl to 5 μl PE-labeled FlowPRA Single HLA Antigen bead panels (FL1HD01 and FL1HD02, OneLambda, Los Angeles, CA), a fixable viability dye (Ghost Dye BV510, Tonbo Biosciences, San Diego, CA), and anti-CD45 e450 (clone HI30, eBioscience, San Diego, CA) for 30 minutes at 37° C. After incubation, the suspensions were washed with DPBS, fixed with 0.5% neutral buffered formalin (VWR International, West Chester, PA), washed again with DPBS, and run in a BD LSRII flow cytometer. Single antigen beads decorated with different HLAs fluoresce in the PE channel with distinct intensity, allowing one to discern the individual HLA alleles. The abundance of unbound beads was quantified in the presence of either A2-CAR Tregs or untransduced Tregs for each single HLA antigen group. Percentage relative binding of A2-CAR Tregs to each HLA allele was then calculated using the following formula

$$\frac{100 \times ( norm . number beads in UT Treg - norm . number beads in CAR Treg )}{norm . number beads in UT Treg}$$

i.e. by dividing the normalized (norm.) number of beads in the untransduced (UT) Treg condition for a specific HLA minus the normalized number of beads in the A2-CAR Treg condition for that same HLA by the normalized number of beads in the untransduced Treg condition, multiplied by 100. HLA antigen bead numbers were normalized using the following formula

$$\frac{200 \times number beads}{number negative control beads}$$

i.e. by multiplying the number of beads of interest in each HLA peak by 200, divided by the number of negative control beads in the sample, to correct for variations in the absolute number of negative control beads acquired in each sample.

Genome engineering: CRISPR/Cas9 genome editing in Tregs and bulk T cells was carried out using ribonucleoprotein (RNP) electroporation as previously described (27). Briefly, RNPs were produced by complexing a two-component guide RNA (gRNA) to Cas9. crRNAs and tracrRNAs were chemically synthesized (Dharmacon, IDT) and Cas9-NLS (nuclear localization signal) was recombinantly produced and purified (QB3 Macrolab). Lyophilized RNA was resuspended at a concentration of 160 μM, and stored in single use aliquots at −80° C. crRNA and tracrRNA aliquots were thawed, mixed 1:1 by volume, and annealed at 37° C. for 30 min. 40 μM recombinant Cas9 was mixed 1:1 by volume with the 80 μM gRNA (2:1 gRNA to Cas9 molar ratio) at 37° C. for 15 min to form an RNP complex at 20 μM. RNPs were electroporated immediately after complexing into Tregs and T cells resuspended in supplemented P3 buffer (Lonza). Guide RNA sequences used for gene editing were:

TRAC:
(SEQ ID NO: 9)
CAGGGTTCTGGATATCTG
TRBC:
(SEQ ID NO: 10)
CCCACCAGCTCAGCTCCACG
HLA-A2:
(SEQ ID NO: 11)
CCTCGTCCTGCTACTCTCGG

Following electroporation, Tregs and T cells were replated for expansion together with the AAV6 containing the A2-CAR homology-directed repair (HDR) template. Alternatively, two days after activation, Tregs were transduced with a lentivirus at a multiplicity of infection (MOI) of 1 by spinoculation for 30 min at 1200 G. The next day, the cells were washed to remove residual virus from the medium and further expanded with recombinant human IL-2 (300 IU/ml). In some instances, A2-CAR⁺ cells were FACS-purified on Day 9 based on MYC-tag expression and the TCR was deleted by electroporating a CRISPR/Cas9 RNP complex targeting the constant region of the TCR beta chain (TRBC).

A2-CAR Treg trafficking to transplant pancreatic iselts: Female or male NSG mice were rendered diabetic by a single intraperitoneal (i.p.) injection of streptozotocin (STZ) at 220 mg/kg and islets were transplanted 72-96 hours later. Blood glucose levels were monitored 2-3 times per week using a glucometer (Nova Max Plus Blood glucose and Ketone Monitor, Nova Diabetes care, Billerica, MA). Only mice with blood glucose levels above 300 mg/dl were used for transplantation. Pancreatic islets from NSG.HLA-A2 transgenic mice (A2-NSG, NOD.Cg-Prkdc^{scid} Il2rg^{tm1Wj1} Tg(HLA-A/H2-D/B2M)1Dvs/SzJ, Jackson Laboratories, Bar Harbor, ME, Stock number 014570) were isolated as previously described (28). Human pancreata were procured from deceased multi-organ donors with research use consents and approval from UCSF institutional review board. Human research islets were isolated by the UCSF Diabetes Center Islet Core following standard protocols (29). A total of either 500 mouse islets or 3000 human islet equivalents (IEQs) were transplanted under the kidney capsule or into the spleen. Blood glucose levels of <200 mg/dl on two consecutive days were defined as successful islet engraftment. Mice that only attained partial graft function (blood glucose range 200-500 mg/dl) by 10 to 14 days after transplant were given subcutaneous insulin pellets (Linbit, LinShin Canada) to support graft function. Luciferase-expressing A2-CAR Tregs or A2-CAR T cells were infused intravenously in STZ-induced diabetic mice transplanted with mouse HLA-A2⁺ islets. Luciferase activity was monitored 2-3 times per week. These animals were anesthetized in an isofluorane chamber, injected i.p. with 100 μl of 15 mg/ml D-Luciferin (Biosynth, Staad, Switzerland) and, 7 min later, imaged in a Xenogen IVIS Spectrum Imaging System (PerkinElmer, Richmond, California). Luciferase data analysis was performed using Living Image software (PerkinElmer).

Xenogeneic graft-vs-host disease (GvHD): NOD.Cg-PrkdC^{scid} Il2rg^{tm1Wj1}/SzJ (NSG) and NOD.Cg Prkdc^{scid} Il2rg^{tm1Wj1}/Tg(HLA-DRB1)31Dmz/SzJ/H2-Ab1tm1Gru×NOD.Cg-Tg(HLA-A/H2-D/B2M)1Dvs/SzJ (A2-NSG) were obtained from Jackson Laboratories. For GvHD induction, animals were irradiated (2.5 Gy) 24 hours prior to retroorbital intravenous (i.v.) infusion of 5×10⁶ freshly isolated PBMCs from either an HLA-A2-positive or an HLA-A2-negative donor with or without 2.5×10⁶ ex vivo expanded third-party A2-CAR Tregs. All mouse experiments were performed according to a UCSF Institutional Animal Care and Use Committee (IACUC) approved protocol.

### Example 1. Development of an HLA-A2-Specific CAR

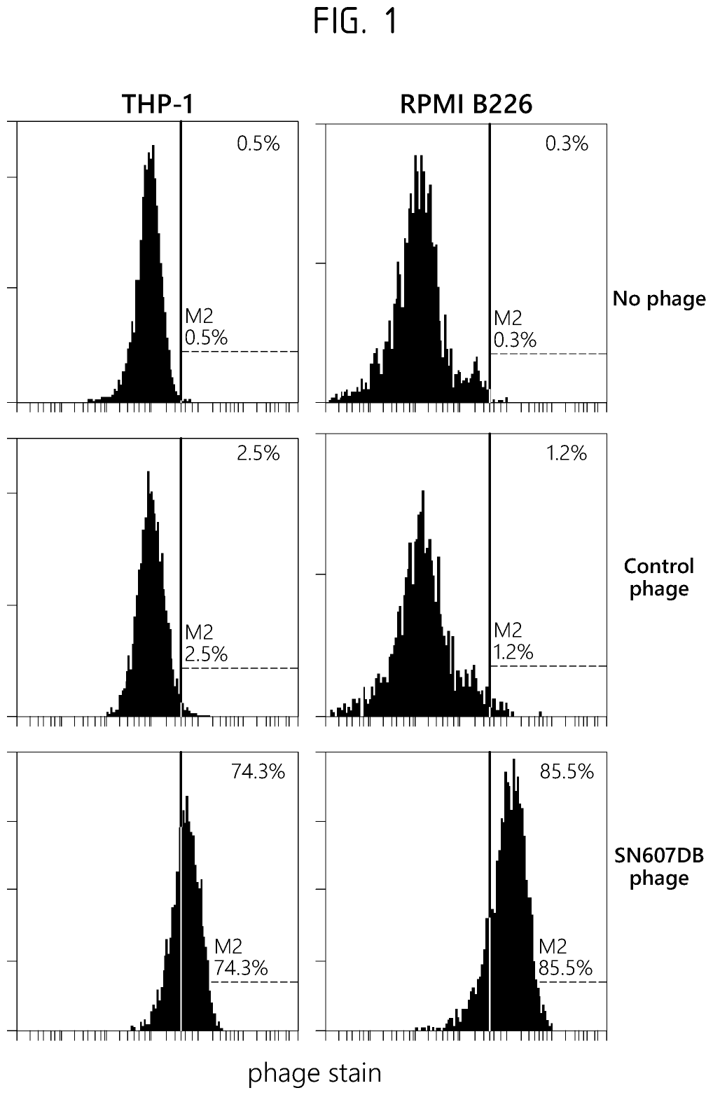

To engineer an anti-A2 CAR, the variable regions of the heavy (V_{H}) and light (V_{L}) chains of an A2-specific IgG1 κ antibody from a hybridoma (SN607D8) produced using B cells isolated from a previously described sensitized donor (16) were first cloned. This antibody was reported to bind to HLA serotypes A2 and A28, which includes HLA-A68 and A69 alleles. After cloning the SN607D8 scFv from the hybridoma, phage-displayed SN607D8 scFv binding to two human tumor cell lines was evaluated. The THP-1 monocytic cell line expresses HLA-A2, but not A28, whereas the RPMI 8226 myeloma cell line is HLA-A2⁻ but has a genotype of HLA-A*6802 and is thus HLA-A28⁺. The results showed that the SN607D8 scFv indeed binds to both cell lines (FIG. 1), demonstrating the retention of the original specificity of the antibody.

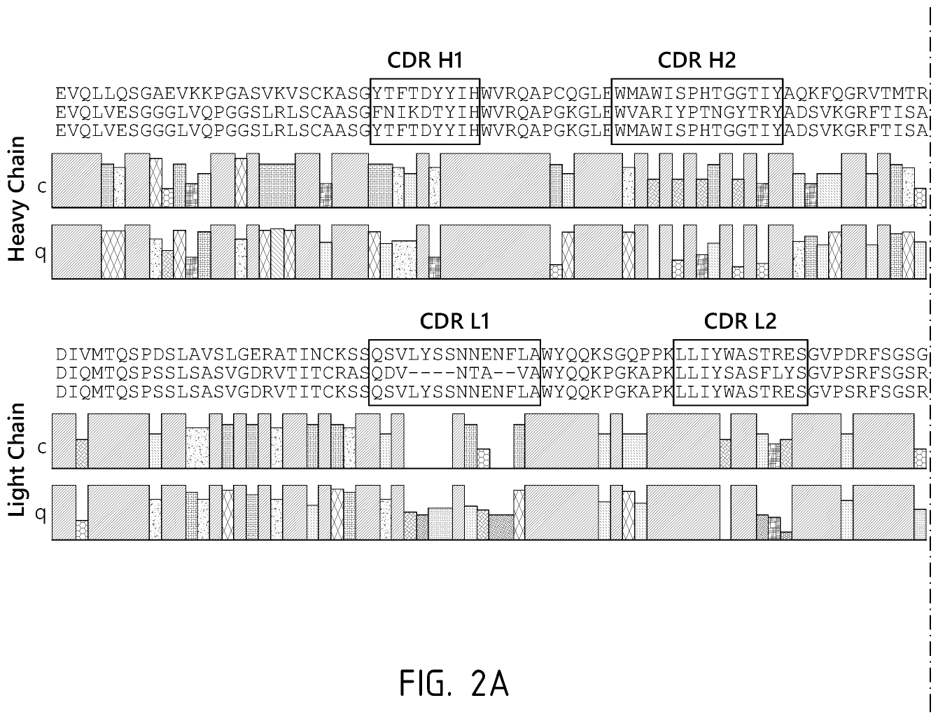

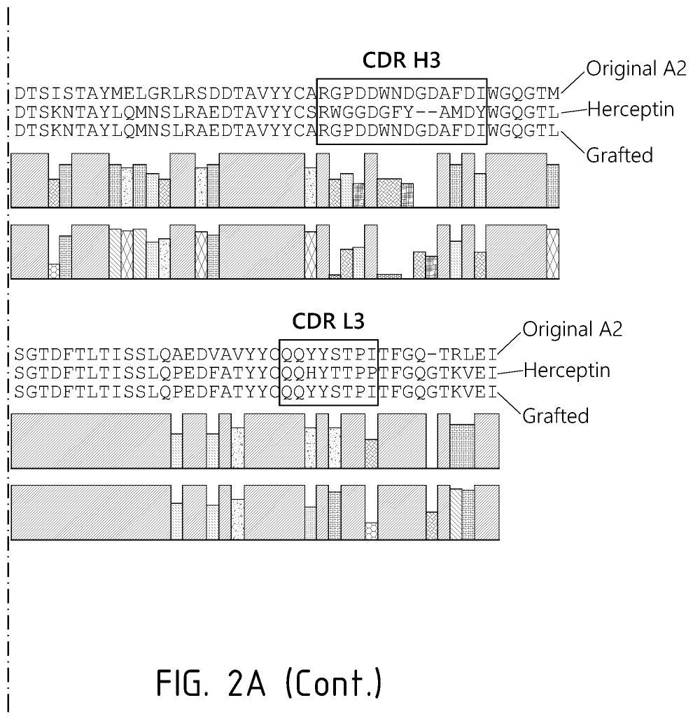

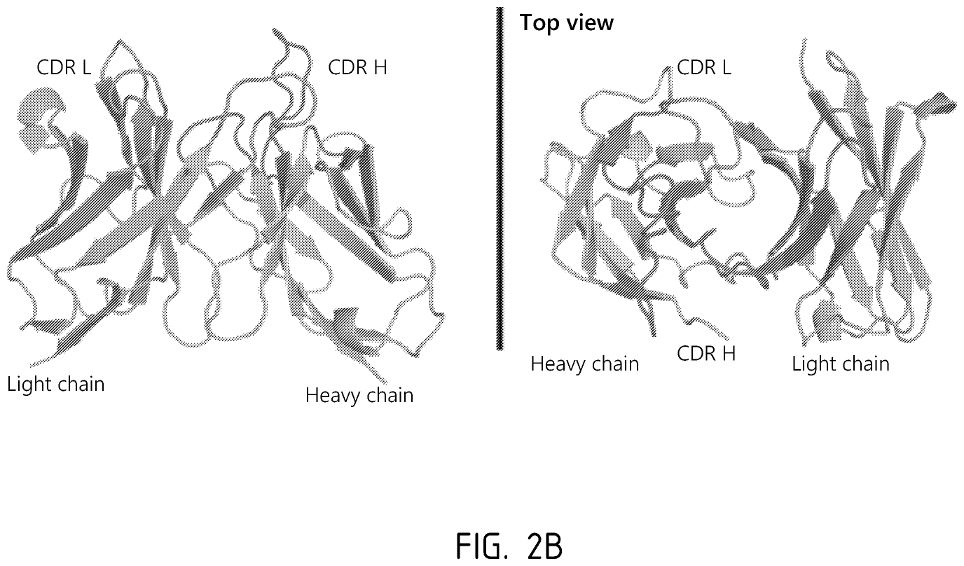

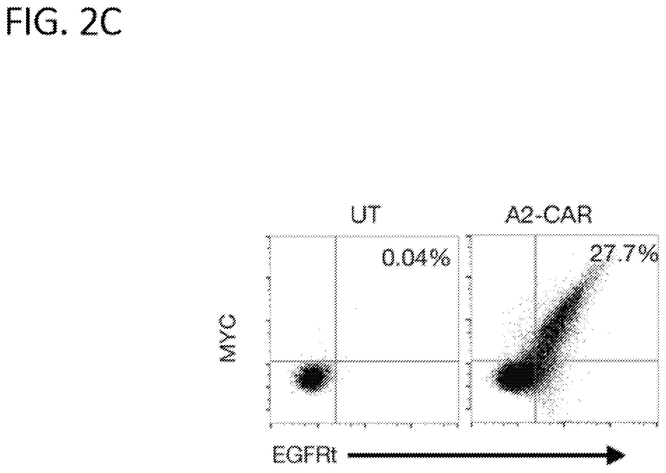

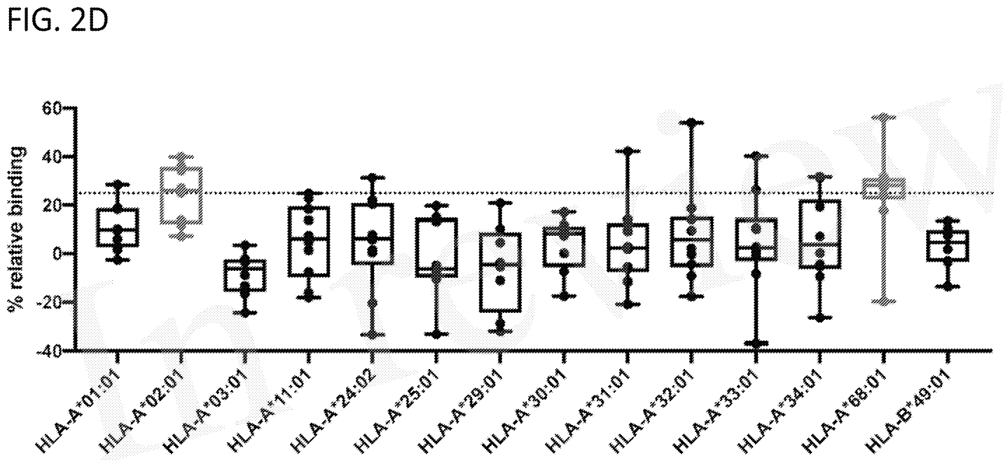

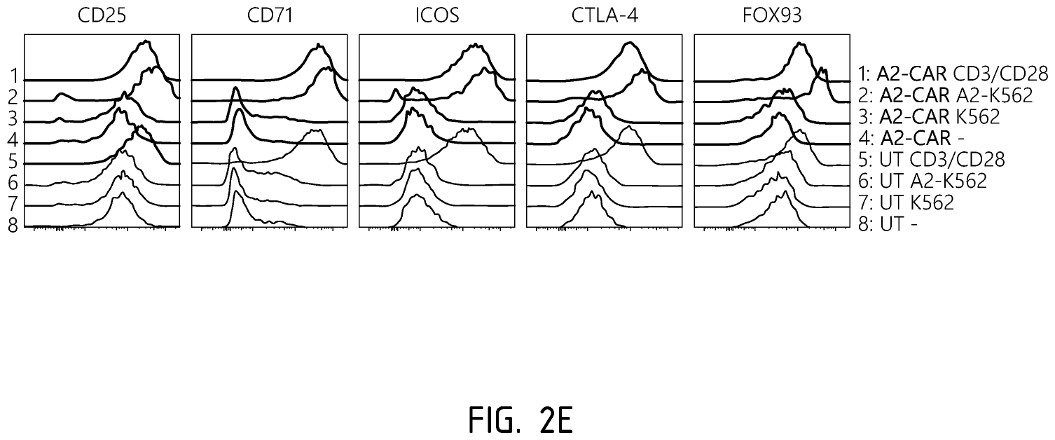

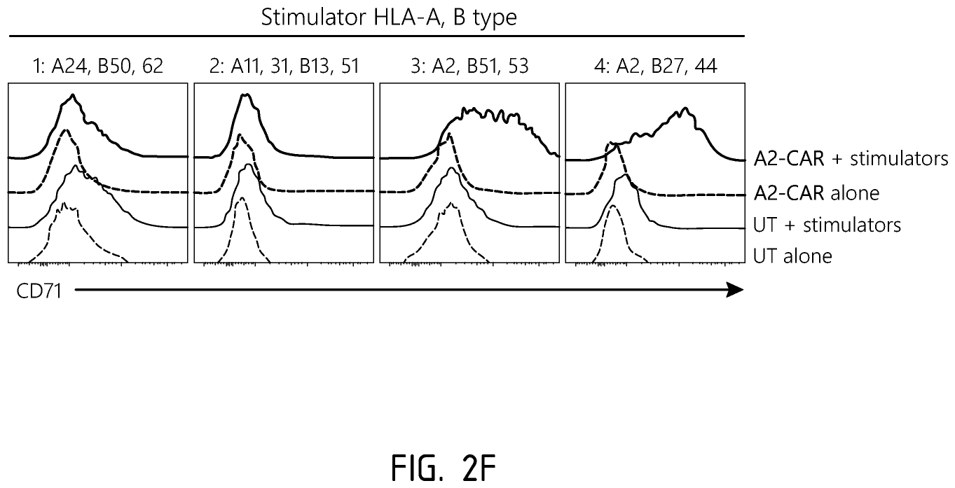

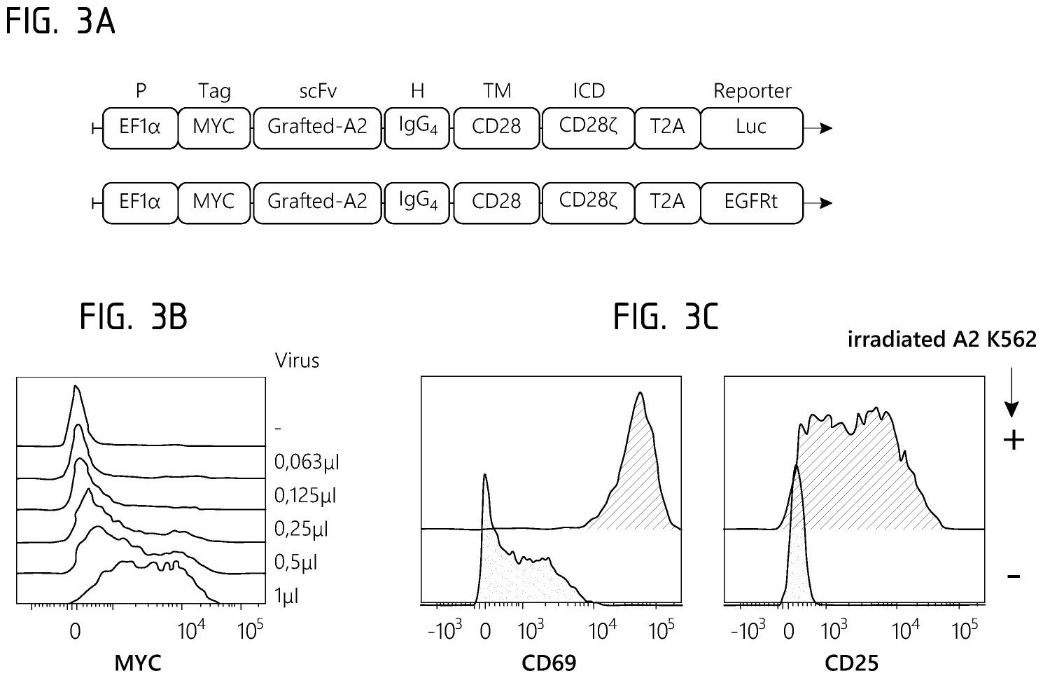

The SN607D8 scFv was then cloned into a construct that contained an IgG4 hinge, the CD28 transmembrane domain, and a signaling domain composed of the CD28 and CD3ζ intracellular domains. Unexpectedly, the CAR failed to express on the surface of human T cells (data not shown). To rescue the expression, the complementarity-determining regions (CDRs) of the heavy and light chains of the SN607D8 scFv were grafted into the framework regions of an scFv derived from the anti-HER2 antibody Herceptin (trastuzumab), which is known to be compatible with CAR surface expression (30). The resulting grafted heavy and light chains FIG. 2A) were connected via a 15 amino acid linker (GGGGS)₃ to form a new grafted scFv, termed QT007YL. Automated computer modeling with an antibody structure prediction tool, ABodyBuilder (31), showed that the grafted scFv folds as expected (FIG. 2B). A new A2-CAR was then generated for expression in human T cells by fusing the QT007YL scFv to an IgG4 hinge, the CD28 transmembrane domain, and a CD28-ζ signaling domain. The resulting A2-CAR was cloned with an N-terminal MYC-tag into a pCDH lentiviral vector behind an EF1α promoter (22). A truncated EGFR (EGFRt) was cloned in-frame behind the CAR separated by a 2A self-cleaving peptide to enable facile evaluation of lentiviral transduction using expression of EGFRt. To enable in vivo tracking of CAR-expressing cells, a version of the lentiviral construct with a luciferase gene behind the CAR separated by a 2A peptide (FIG. 3A) was also generated. HLA-A2-negative Jurkat T cells were first transduced to assess the expression and function of the grafted A2-CAR. Detection of the MYC-tag via flow cytometry verified efficient surface expression of the A2-CAR (FIG. 3B). Lentiviral transduction of primary human Tregs with the grafted A2-CAR also resulted in co-detection of the MYC-tag and EGFRt on the cell surface (FIG. 2C). Moreover, A2-CAR Jurkat T cells upregulated CD69 and CD25 expression specifically when co-cultured with irradiated HLA-A2⁺ K562 tumor cells (FIG. 3C), suggesting that the grafted A2-CAR is able to activate T-cell signaling in response to HLA-A2.

The HLA-A2 molecule contains many polymorphic eplets that are shared with other HLA class I molecules (Table 2).

TABLE 2
Polymorphic eplets shared between HLA-A2 and other HLA class I alleles.

| | Amino acid | | |
|---|---|---|---|
| Eplet | residues | Shared with | Reference |
| 44RME | 44R, 45M, 46E | A3, A11, A23, A24, A25, A26, A29, A30, | (16) |
| | | A31, A32, A33, A34, A43, A66, A68, A69, | |
| | | A74, A80 | |
| 62GE | 62G63E | B57, B58 | (66) |
| 105S | 105S | A3, A23, A24, A29, A30, A31, A33, A34, | (16) |
| | | A68, A69, A80 | |
| 127K | 127K | A23, A24, A68, A69 | (46) |
| 144TKH | 142T, 144K, 145H | A68, A69 | (32) |
| 145KHA | 144K, 145H, 149A | A68, A69 | (46) |

The parental monoclonal antibody SN607D8 used to generate the grafted A2-CAR has a specificity for the eplet 144TKH (142T, 144K, and 145H residues), which is shared between HLA-A2, -A68, and -A69, but not with HLA-A3, -A11, or -A24 (32). To verify that the grafted A2-CAR retained the specificity for 144TKH, binding of grafted A2-CAR transduced Tregs to a FlowPRA Single Antigen bead panel was tested. In this assay, CAR specificity is defined as an increase in binding to HLA-bearing beads over control beads of at least 25%, a threshold used to define the binding specificity of a previously reported A2-CAR (26). The grafted A2-CAR reacted with HLA-A2 and HLA-A68, but not HLA-A3, -11, or -24 (FIG. 2D). To determine if A2-CAR can mediate in vitro activation of human Tregs, A2-CAR Tregs were co-cultured with either HLA-A2⁺ or HLA-A2⁻ target cells (FIG. 2E). A2-CAR Tregs upregulated CD25, CD71, ICOS, FOXP3, and CTLA4 48 hours after stimulation with HLA-A2⁺, but not with HLA-A2-target cells, demonstrating the HLA-A2 reactivity of the QT007YL A2-CAR. Finally, in vitro response of A2-CAR T cells to cells expressing HLA-A2, HLA-A11, HLA-A24, or HLA-A31 were evaluated. Only HLA-A2⁺ cells, not HLA-A11, 24, or 31⁺ cells, were able to activate A2 CAR, QT007YL A2-CAR did not recognize 44RME, 105S, or 127K eplets (FIG. 2F). Together these results show that the specificity of the parental antibody was preserved in the QT007YL scFv after grafting.

### Example 2. In Vivo Trafficking of Monospecific A2-CAR T Cells in an Islet Transplantation Model

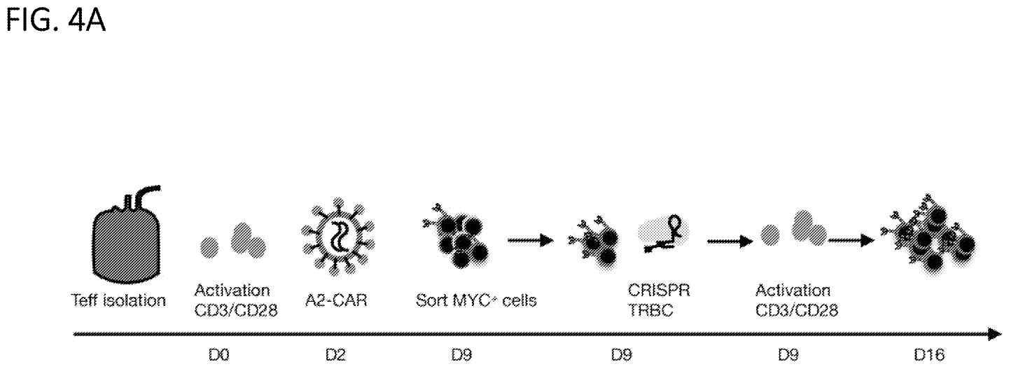

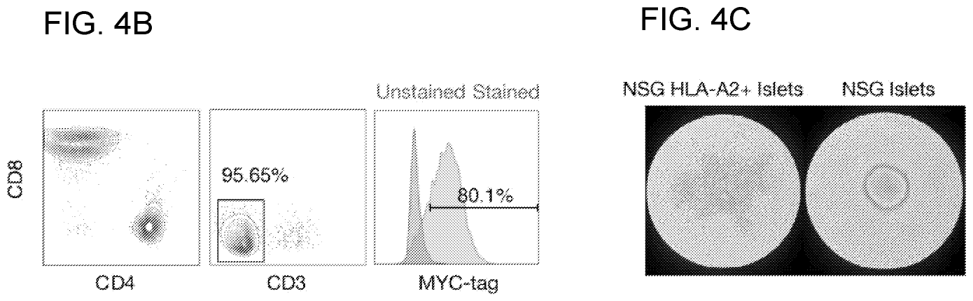

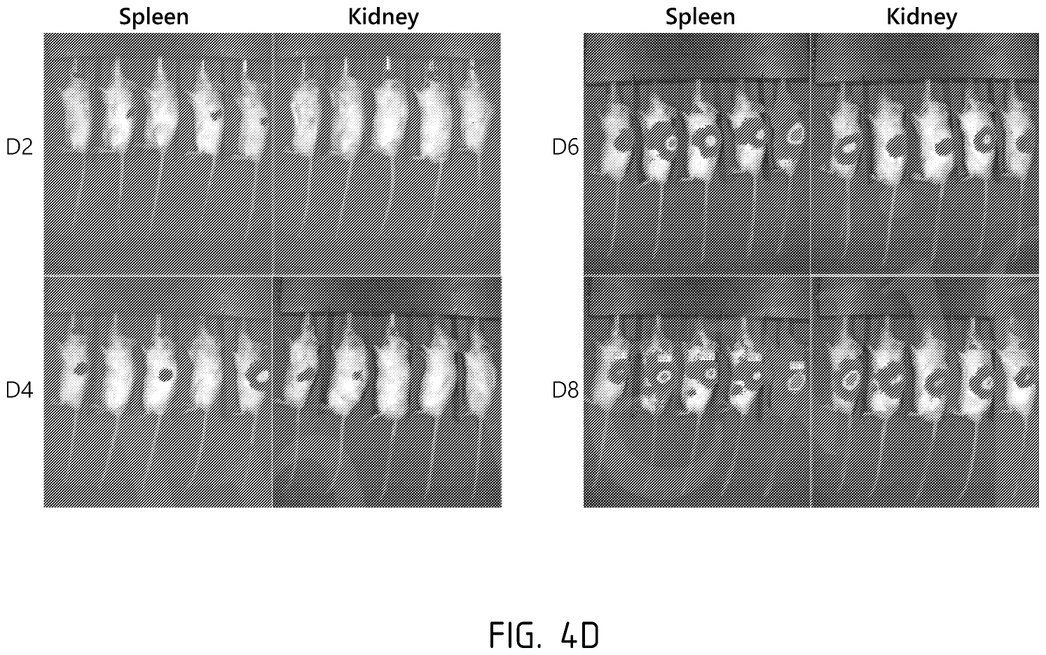

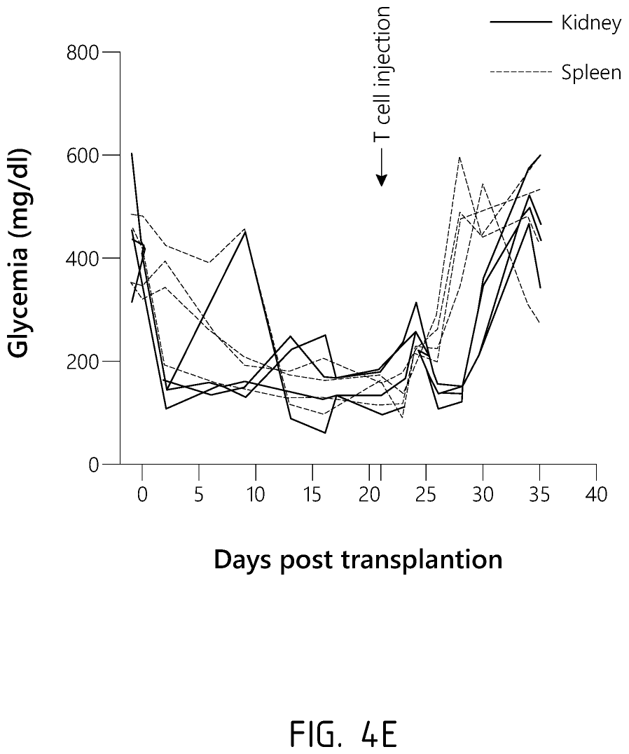

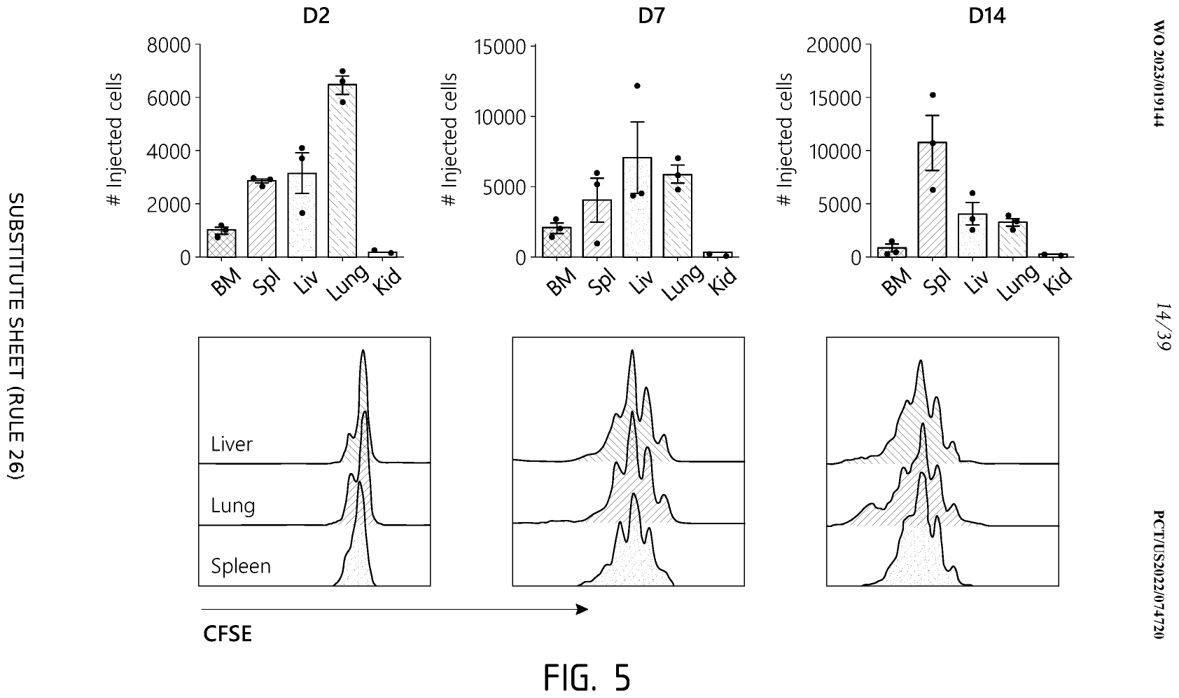

Next, QT007YL A2-CAR-expressing T cells were injected in NSG mice to determine whether CAR expression could redirect T cells to HLA-A2-expressing tissues in vivo. A significant fraction of human T cells can recognize mismatched HLA and trigger rejection of transplanted allogeneic human tissue in NSG mice (33). Additionally, human T cells have conspicuous reactivity against xenogeneic antigens expressed in the mouse host, with the potential to divert T cells away from human grafts and also eventually cause GvHD (34). To avoid these confounding issues, primary human T cells were first lentivirally transduced to express the A2-CAR. A₂-CAR^{+TCRdeficient} T cells were subsequently generated by CRISPR/Cas9-mediated knockout of endogenous TCR expression from Day 9 FACS-purified A2-CAR⁺ cells (FIG. 4A). Because pre-existing TCRs were still expressed on the cell surface after gene deletion, the edited cells were able to be expanded by restimulation with anti-CD3/CD28 beads for another 7 days, resulting in 95.65% CD3⁻ and 80.1% MYC-tag⁺ cells (FIG. 2B). Co-culturing these ^{TCRdeficient} A2-CAR T cells with islets from HLA-A2 transgenic NSG (A2-NSG) or WT NSG mice for 48 hours resulted in the selective destruction of A2-NSG transgenic mouse islets (FIG. 4C), demonstrating that the grafted A2-CAR can be specifically activated by HLA-A2 molecules expressed on islet tissue in vitro. To determine if ^{TCRdeficient} A2-CAR T cells can recognize A2-expressing islets in vivo, transplanted HLA-A2 transgenic mouse islets were first transplanted into STZ-induced diabetic NSG mice, and then the mice were infused with 2×106 ^{TCRdeficient} A2-CAR T cells after the islet grafts had been established. Kidney capsule is a standard site for islet transplantation. However, human CD4⁺ T cells efficiently trafficked to the lungs, livers and spleens, but not kidneys of NSG recipients (FIG. 5). Thus the islets were transplanted either into the spleen or under the left kidney capsule and luciferase-expressing A2-CAR T cell migration was monitored using bioluminescence imaging. A progressive increase in luciferase signal was observed in the recipient mice, although it was difficult to discern the accumulation of A2-CAR T cells in the spleen versus the left kidney (FIG. 4D). However, A2-NSG islets were rejected in both groups, with a median survival of 6 days in the spleen and 11 days under the kidney capsule (FIG. 4E). This demonstrated that the A2-CAR was able to direct the trafficking and accumulation of human T cells to sites of antigen deposition outside the route of T cell migration.

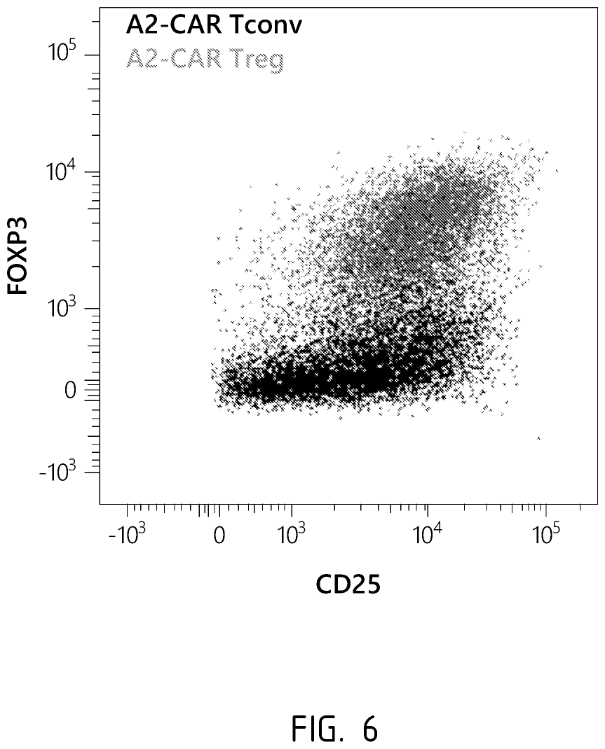

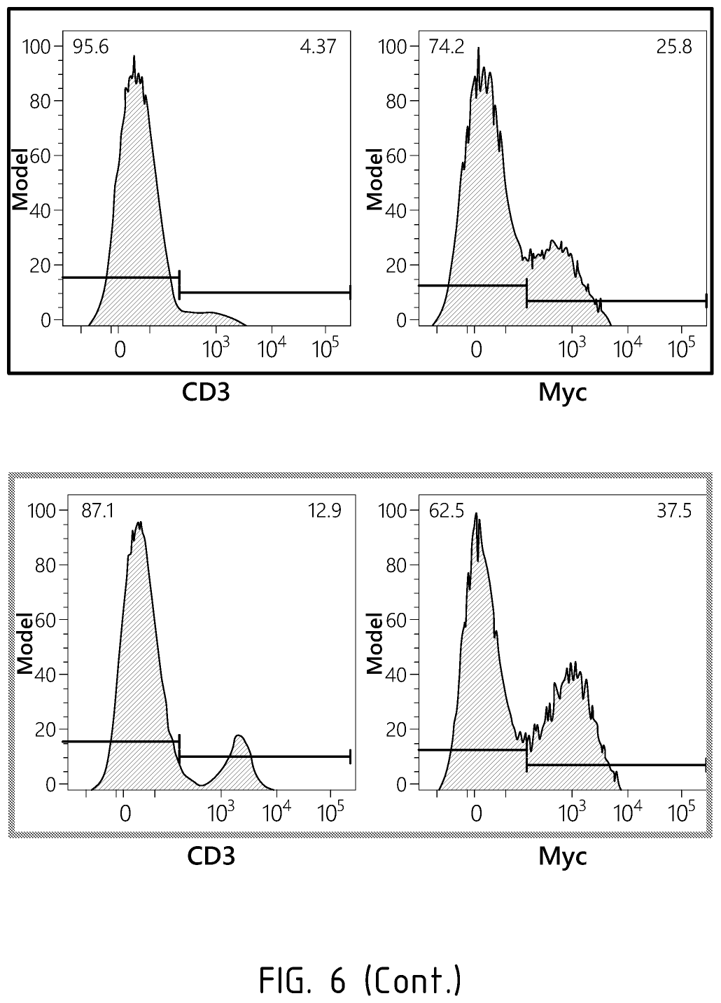

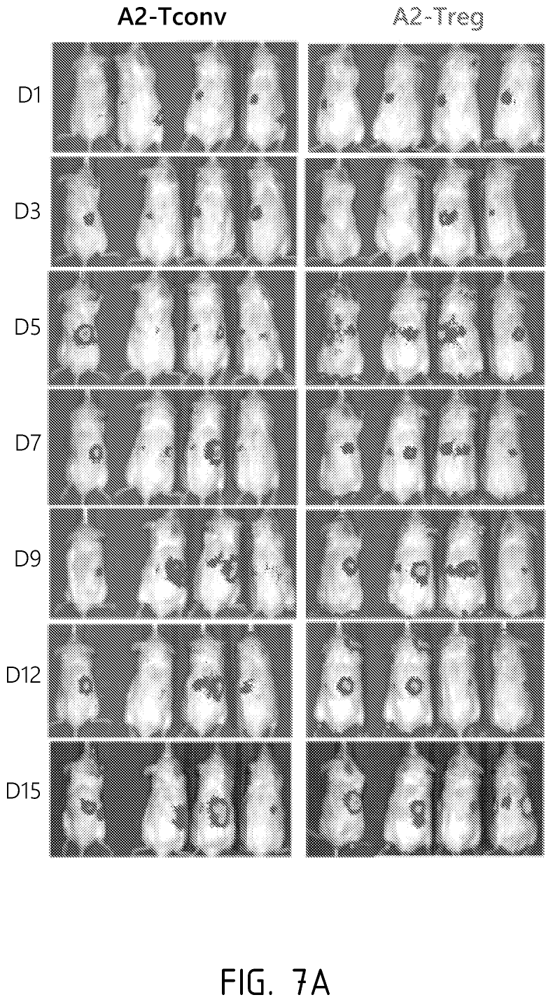

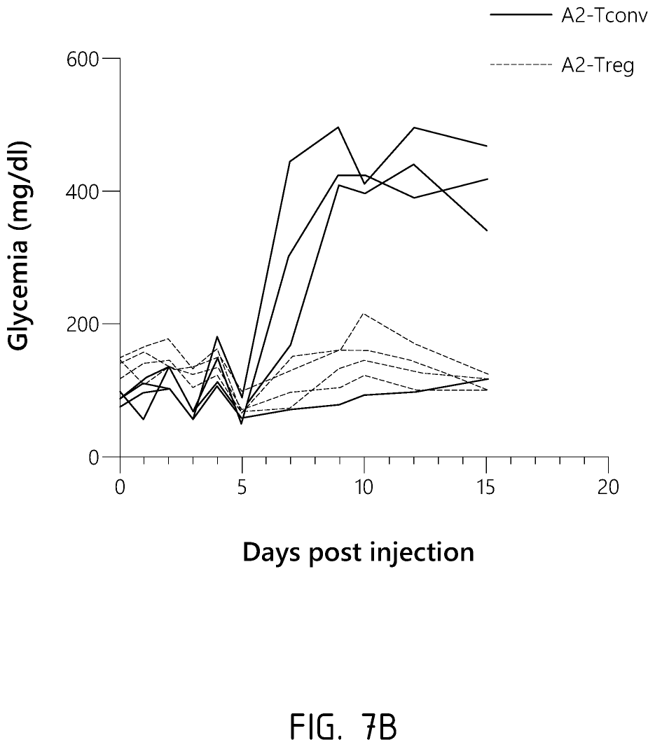

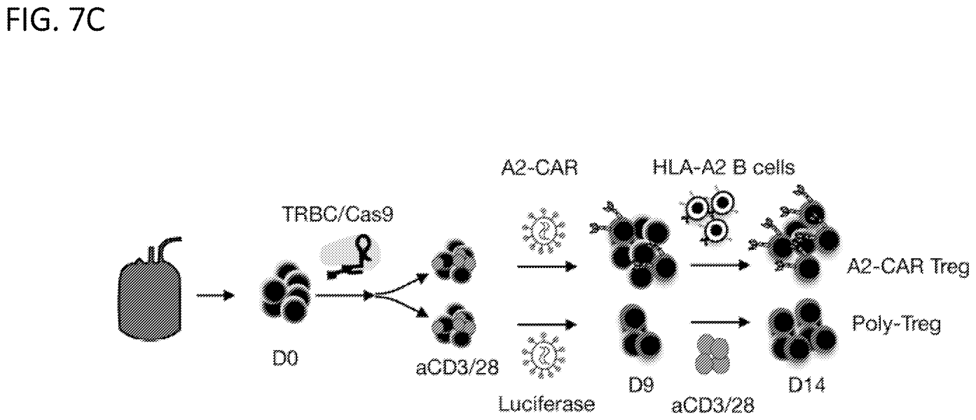

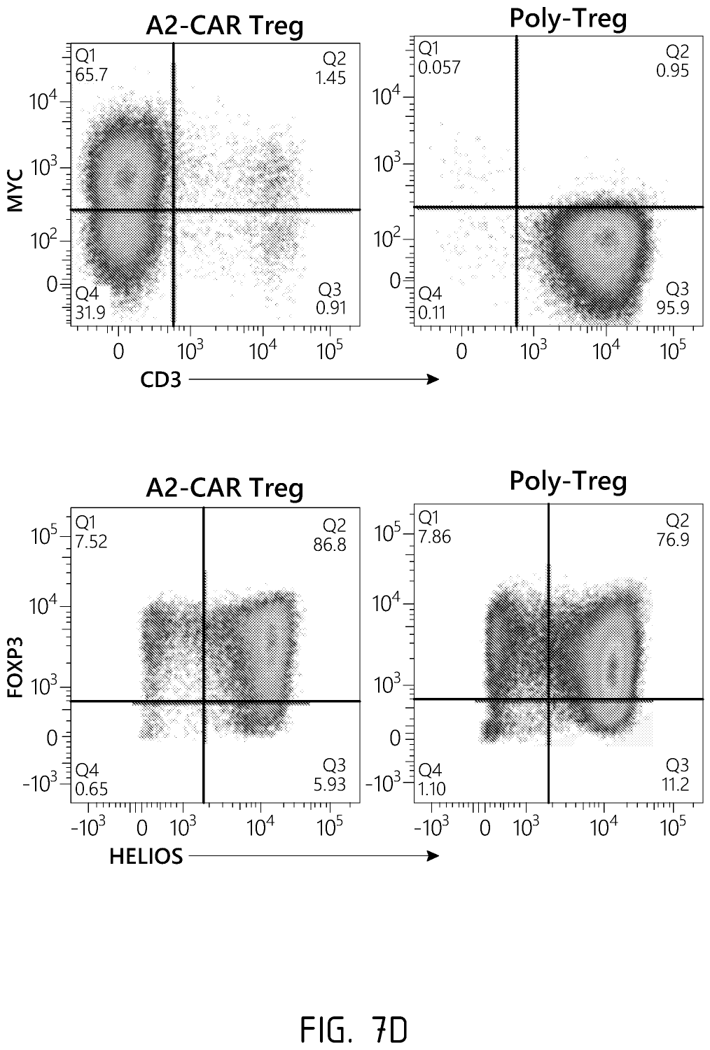

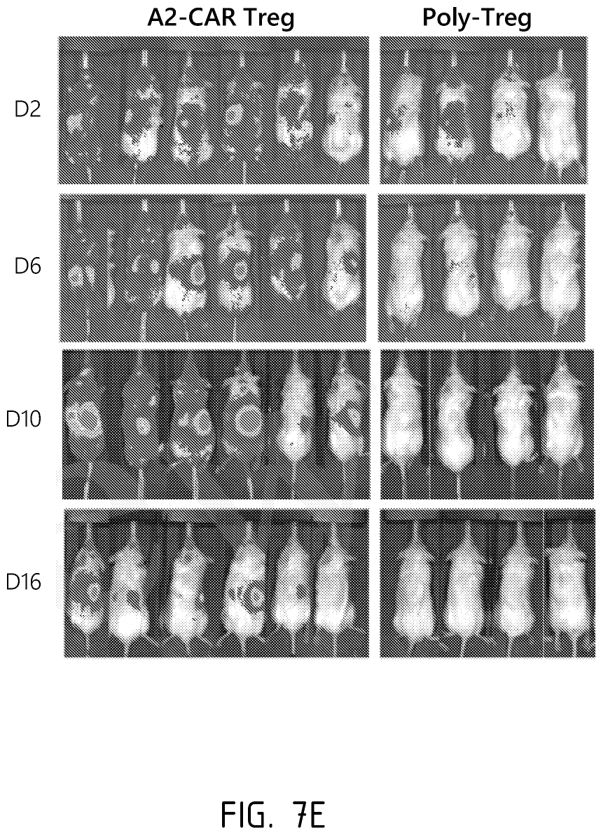

The kidney capsule islet transplantation model was then used to evaluate the in vivo trafficking of A2-CAR Tregs. Luciferase-labeled human ^{TCRdeficient} A2-CAR Tregs were generated as described in FIG. 4A and the resulting cells expressed Treg lineage markers FOXP3 and CD25 (FIG. 6). These cells were then infused into STZ-induced diabetic animals transplanted with A2-NSG transgenic islets. In this experiment, the islets were transplanted under the right kidney capsule to enable greater spatial separation from the spleen during bioluminescence imaging. To support Treg persistence in the absence of human IL-2-producing cells, mice infused with 2×10⁶ A2-CAR Tregs received daily i.p. injections of recombinant human IL-2 (50,000 IU/day/mouse). For comparison, a cohort of mice was separately infused with 2×10^{6 TCRdeficient} A2-CAR conventional CD4⁺ T cells (Tconv), without IL-2 injection. In both groups, luciferase activity was observed first in the spleen and 3-7 days later in the right kidney (FIG. 7A), demonstrating that both A2-CAR Treg and Tconv cells can traffic to the A2⁺ grafts. Importantly, islet rejection was observed in 3/4 mice that received A2-CAR CD4⁺ Tconv cells, but 0/4 mice that received A2-CAR Tregs (FIG. 7B). Despite accumulation within the grafts, the lack of mouse islet destruction by the human A2-CAR Tregs suggests that CAR Tregs do not have overt toxicity against islets.

To validate that A2-CAR Tregs can traffic to human A2+ islets, this experiment was repeated with human HLA-A2⁺ islets transplanted under the right kidney capsule. In this experiment, human Tregs were first treated with CRISPR/Cas9 ribonucleoprotein (RNP) complexes targeting the TCR beta constant (TRBC) locus to eliminate endogenous TCR expression prior to lentiviral transduction with an A2-CAR-2A-luciferase construct. To expand sufficient numbers of ^{TCRdeficient} A2-CAR Tregs, cells were re-stimulated with HLA-A2⁺ stimulated B cells (sBCs) on Day 9 of culture for an additional 5 days (FIG. 7C). As a control, TCR-unedited polyclonal Tregs were transduced with a luciferase-only construct and restimulated with anti-CD3/28 beads on Day 9. Tregs maintained FOXP3 and HELIOS expression prior to infusion (FIG. 7D). Mice received 2×10⁶ Tregs intravenously with subsequent daily i.p. IL-2 injections. A2-CAR-expressing, but not polyclonal Tregs, trafficked from the spleen to the right kidney (FIG. 7E). Together, these results demonstrate that TCR^{deficient}A2-CAR Tregs efficiently traffic to and accumulate in human HLA-A2⁺ islets in vivo.

### Example 3. Knocking the A2-CAR into the TRAC Locus of Tregs

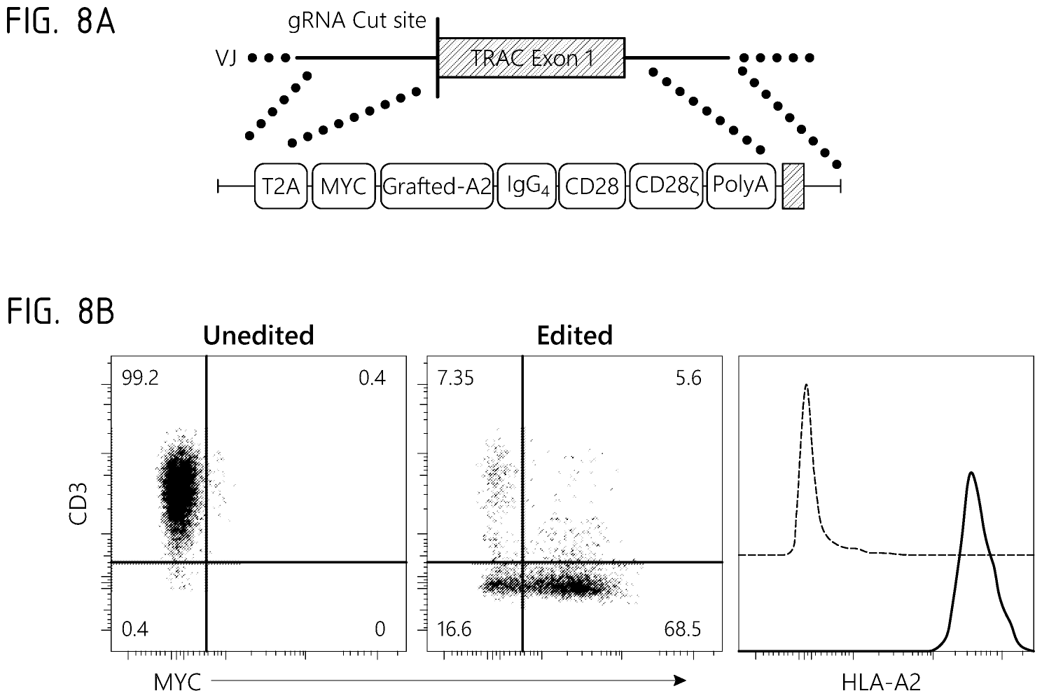

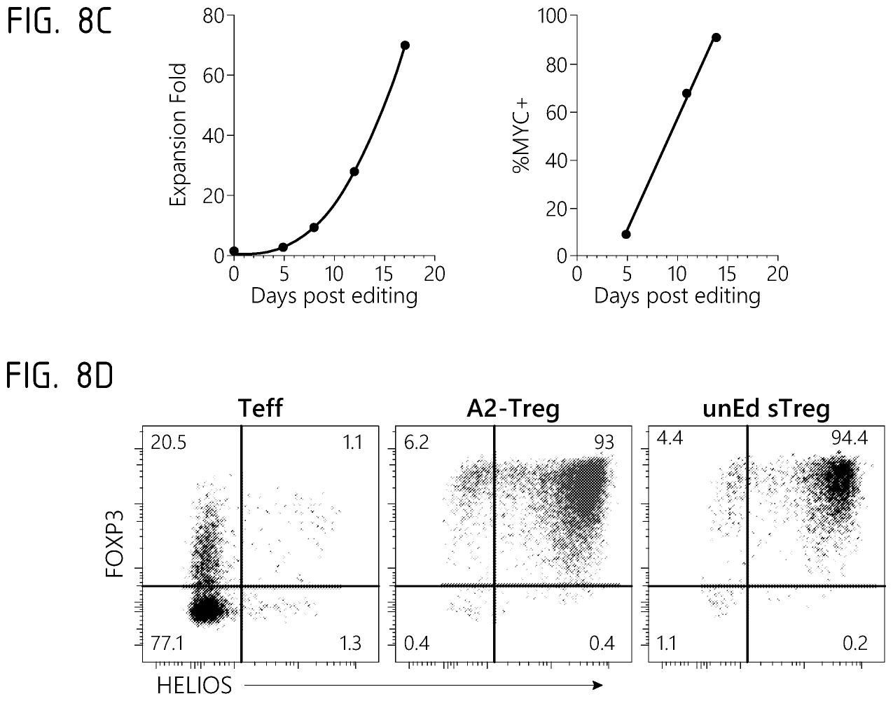

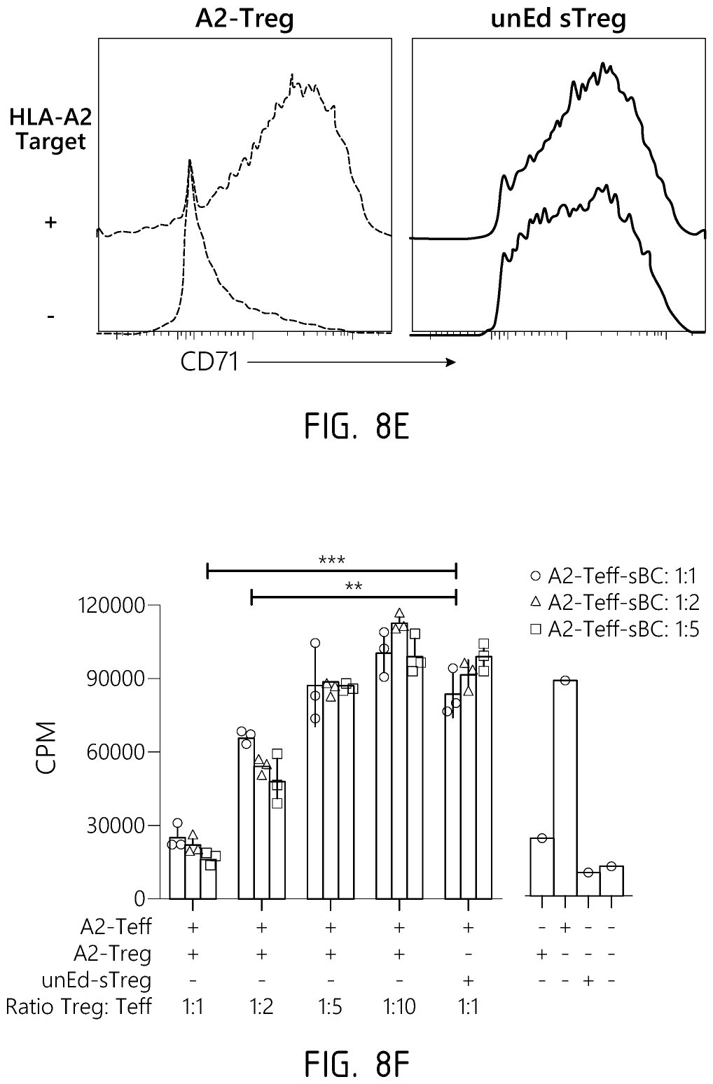

To achieve more uniform A2-CAR expression and investigate its function independently of the endogenous TCR, homology-directed repair (HDR) was employed to site-specifically integrate the A2-CAR into the TCR alpha constant (TRAC) locus, replacing expression of the endogenous TCR with expression of the A2-CAR (FIG. 8A). CD4⁺CD25^{high}CD127^{low} Tregs were FACS-purified and activated with anti-CD3/CD28 beads in the presence of IL-2 (300 IU/ml). Two days later, anti-CD3/CD28 beads were magnetically removed, and the cells were electroporated with Cas9-gRNA ribonucleoprotein (RNP) complexes targeting the TRAC and transduced with AAV6 encoding the QT007YL A2-CAR HDR template. Because this blood donor happened to be HLA-A2⁺, we also included Cas9-gRNA RNP designed to target the HLA-A2 gene locus. TRAC and HLA-A2 gene knockout efficiencies were approximately 85% and 95%, respectively. A minor (5%) population of MYC-tag⁺TCR⁺ cells was observed, likely resulting from incomplete TRAC inactivation and either monoallelic A2-CAR genomic integration into the other TRAC locus or off-target integration (FIG. 8B). Importantly, while the percentage of A2-CAR-expressing MYC-tag⁺ (edited) Tregs was initially low (9%), A2-CAR^{+TCRdeficient} Tregs preferentially expanded in vitro, presumably due to activation by residual HLA-A2 surface expression shortly after CRISPR/Cas9-mediated HLA-A2 gene knockout. In the absence of further exogenous stimulation, 91% of cells were MYC-tag⁺ after 14 days (FIG. 8C).

On Day 14 of culture, FOXP3 and HELIOS expression was evaluated among edited A2-CAR^{+TCRdeficient} Tregs, unedited CD4⁺ Tconv, and unedited Tregs (unEd sTreg, stimulated by anti-CD3/CD28 beads on Days 0 and 9, as per standard protocol for polyclonal Treg expansion (35)). Over 99% of A2-CAR^{+TCRdeficient} Tregs were FOXP3 positive and 93% were HELIOS and FOXP3 double positive (FIG. 8D). Co-culture of the A2-CAR^{+TCRdeficient} Tregs with NALM6, an HLA-A2-positive B cell-derived leukemia cell line, led to a marked and specific upregulation of surface CD71 expression, demonstrating the antigen-driven activation of the edited cells (FIG. 4E). Finally, the suppressive function of the A₂-CAR^{+TCRdeficient} Tregs was evaluated in vitro by co-culturing them with A₂-CAR^{+TCRdeficient} T cells and HLA-A2⁺ irradiated sBCs and assessing T cell proliferation. A2-CAR^{+TCRdeficient} Tregs suppressed the proliferation of A2-CAR^{+TCRdeficient} T cells in the presence of HLA-A2⁺ sBCs, whereas unedited polyclonal Tregs did not (FIG. 8F).

### Example 4. Monospecific A2-CAR Treg Function In Vivo in Xenogeneic GvHD Models

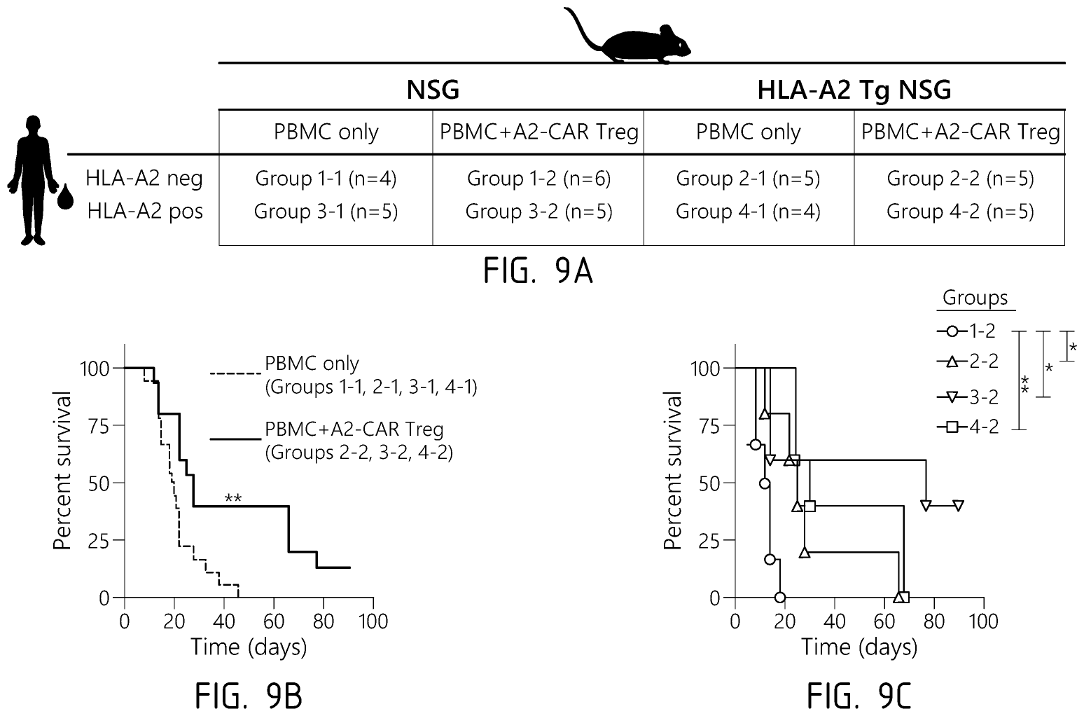

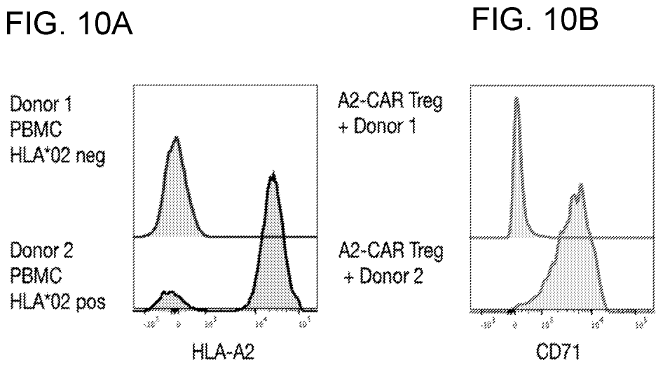

The in vivo functionality of A2-CAR^{+TCRdeficient} Tregs was next tested in vivo within models of xenogeneic GvHD, induced by human PBMCs in sub-lethally irradiated NSG mice. In these experiments, GvHD was induced by infusing PBMCs from an HLA-A2⁺ or an HLA-A2-donor into HLA-A2-transgenic or wild-type (i.e. lacking HLA-A2 expression) NSG mice (FIG. 9A, FIG. 10A). This created 4 experimental groups with regards to the expression of HLA-A2: 1 absent; 2 expressed by the NSG recipients, 3 expressed by the infused PBMCs; and 4 expressed by both NSG recipients and PBMCs. A subset of mice in each of the 4 groups also received A2-CAR^{+TCRdeficient} Tregs at the time of PBMC infusion. It was first confirmed in a mixed lymphocyte reaction (MLR) that A₂-CAR^{+TCRdeficient} Tregs upregulated CD71 only in the presence of the PBMCs from the HLA-A2⁺ donor used for the immune reconstitution (FIG. 10B). To avoid direct contact between the PMBCs and Tregs during infusion, the cells were injected separately into contralateral retro-orbital plexus. A₂-CAR^{+TCRdeficient} Tregs delayed GvHD in mice that had HLA-A2 expressed by the PBMCs, the NSG recipients, or both (FIG. 9B) and failed to confer any protection against GvHD in wild-type NSG animals reconstituted with HLA-A2⁻ PBMC, with a median survival of 13 days (FIG. 9C). This demonstrates that A2-CAR Tregs can function in the absence of the endogenous TCR and that A2-CAR Treg-mediated protection from GvHD depends on the presence of the HLA-A2 antigen.

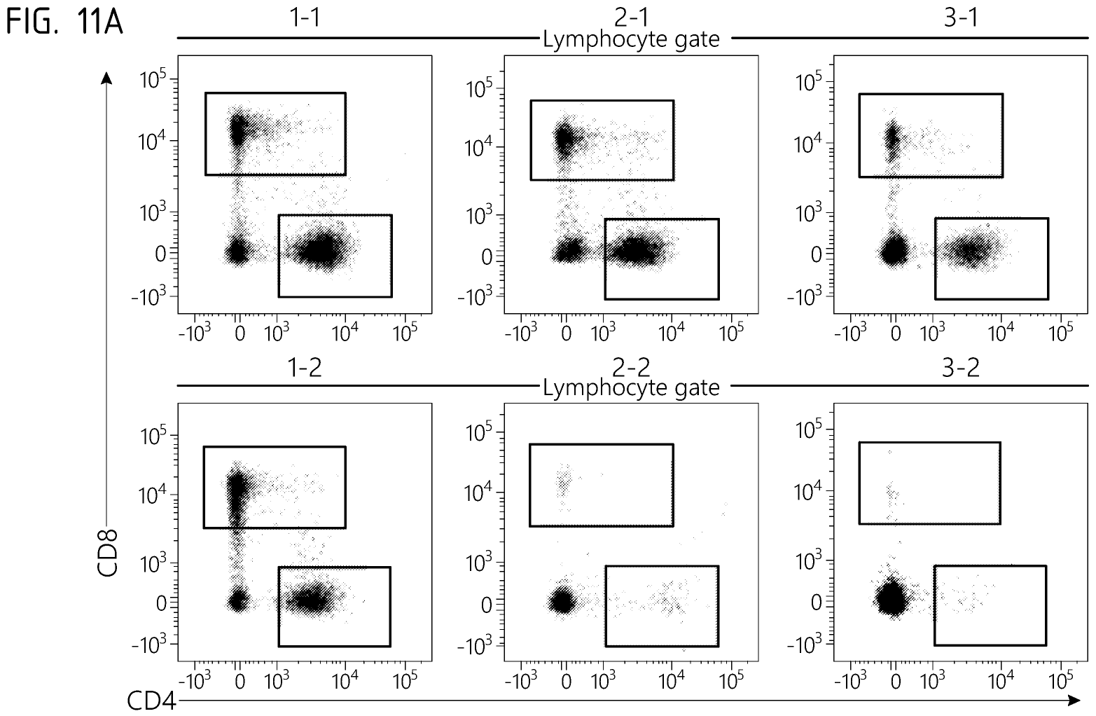

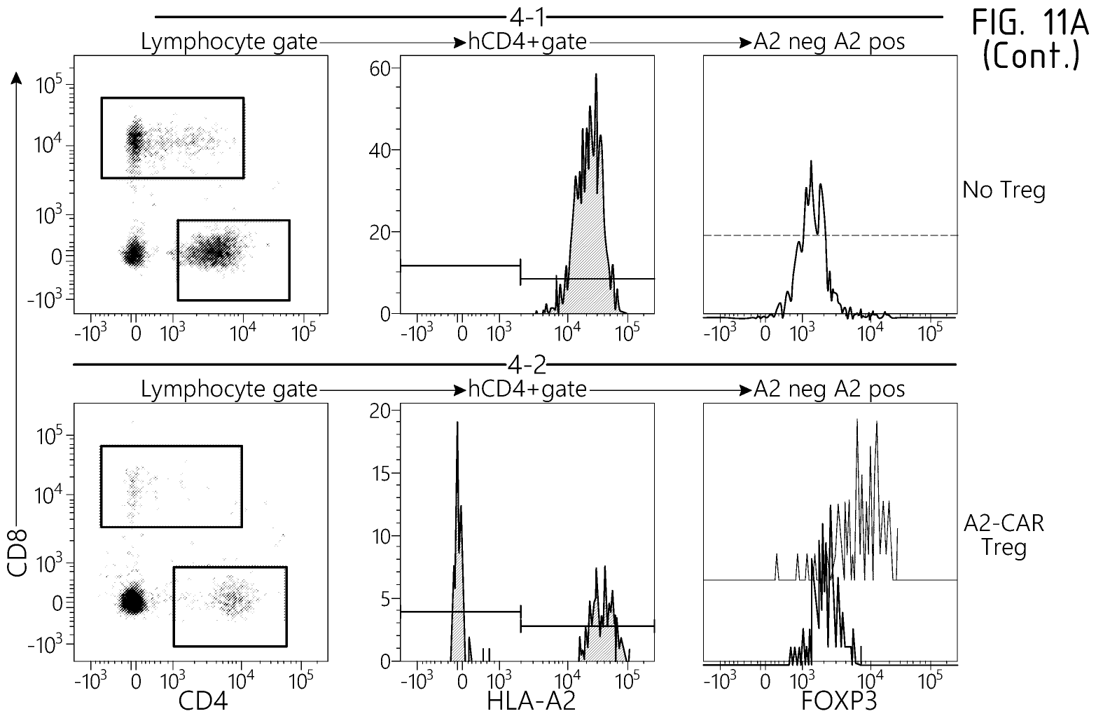

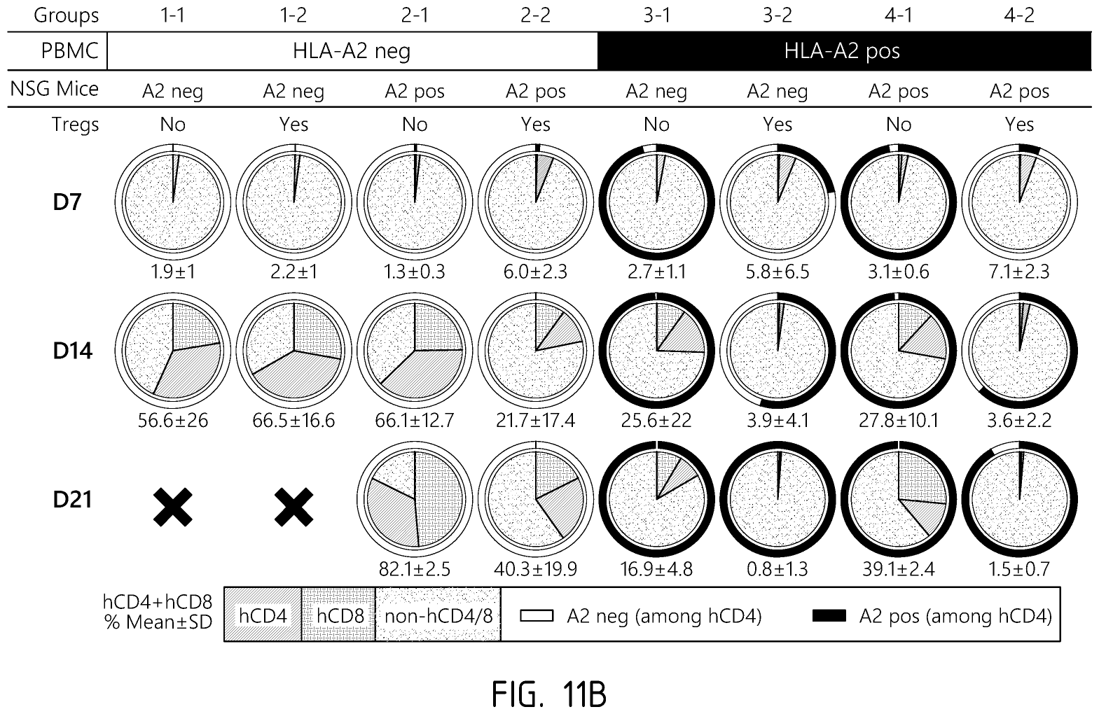

To further investigate the mechanism of A₂-CAR^{+TCRdeficient} Treg-mediated protection, the percentage of circulating human CD4⁺ and CD8⁺ T cells in the peripheral blood of treated animals at Days 7, 14, and 21 following cell injection was determined (FIG. 11A). As previously reported (10), it was observed that HLA-A2⁺ T cells failed to engraft in mice that also received A2-CAR^{+TCRdeficient} Tregs, irrespective of HLA-A2 expression by the host mice (FIGS. 11A-I1B). Meanwhile, the frequency of engrafted HLA-A2⁻ T cells was vastly reduced, but not completely eliminated, when co-injected with A₂-CAR^{+TCRdeficient} Tregs in HLA-A2⁺ transgenic host mice. In HLA-A2 transgenic mice reconstituted with HLA-A2⁺ PBMCs (groups 4-1 and 4-2), it was found that the circulating HLA-A2⁻ cells, i.e. the engineered A2-CAR^{+TCRdeficient} Tregs, remained FOXP3⁺ (FIG. 11A), albeit with limitations in the number of acquired events due to the marked decrease in the number of CD4⁺ and CD4⁺HLA-A2⁻ cells over time (FIG. 11B).

### DISCUSSION

Here, the successful development of a novel human anti-HLA-A2 CAR is reported. Two other human and humanized A2-CARs have been previously described: one by the group of Megan Levings, where a mouse anti-A2 BB7.2 hybridoma was humanized (26), and a second by the groups of Giovanna Lombardi and Elmar Jaekel, generated from a previously published anti-HLA-A2 antibody sequence (36) (clone 3PB2 V_{H} and DPK1 V_{L}) derived from a sensitized blood transfusion patient (11, 12). The original anti-HLA-A2 hybridoma (SN607D8), first described in 2003, was isolated from a woman sensitized during her pregnancies (16). Its HLA specificity was determined by complement-dependent cytotoxicity on a large panel (n>230) of HLA-typed peripheral blood lymphocytes (37), where it was found to only cross-react with HLA-A28, a split antigen that encompasses HLA-A68 and HLA-A69 alleles. The epitope responsible for this cross-reactivity has also been pinpointed, being defined by the amino acids 142T/145H (38). Such extensive characterization was instrumental for us to confirm the preservation of the specificity of the original and the grafted A2-CAR scFv.

The initial failure to express an A2-CAR constructed with an scFv derived from the original SN607D8 hybridoma on the surface of human T cells suggests possible conformational instability. The scFv CDR regions were then grafted into an scFv framework (trastuzumab) known to be compatible with CAR surface expression (30). Thus, the trastuzumab framework may confer greater stability to scFvs for CAR protein folding and expression. However, it should be noted that this grafting strategy has not been tested with other scFvs, and thus cannot be certain of the broader applicability of this CDR-grafting approach. Nevertheless, success in grafting the specificity of the SN607D8 antibody shows that this approach may be useful when designing CARs for new targets.

It is currently unknown whether the endogenous TCR impacts the function of CAR Tregs. Thymically derived Tregs have a highly diverse TCR repertoire that is skewed towards recognizing autoantigens (39), and work in mice has demonstrated that Tregs require continuous TCR signaling to maintain normal immune homeostasis (40). Thus, retaining the TCR in CAR Tregs might support their homeostasis in vivo. However, in the context of solid organ transplantation, A2-CAR Tregs traffic to the HLA-A2-expressing graft, as shown in previous work (11, 12) and in this study, thus receiving continuous signaling via the CAR, ultimately inducing bystander suppression and supporting homeostasis independently from their endogenous TCR. The islet transplantation experiments show that A2-CAR Tregs with or without endogenous TCR efficiently traffic to the site of antigen expression. Moreover, the finding that TCR-deficient A2-CAR⁺ Tregs suppress GvHD, in an HLA-A2-dependent manner, shows that CAR Tregs can function without their endogenous TCR.

The results are consistent with previous work that has shown efficient GvHD prevention by A2-CAR Tregs in NSG mice infused with HLA-A2⁺ PBMCs (10). By analyzing PBMC engraftment, it was apparent that the protection from GvHD was a result of preventing T cell engraftment, possibly due to direct recognition of HLA-A2⁺ PBMCs by A2-CAR Tregs. Low T cell engraftment is not a desirable outcome of Treg therapy for GvHD, as immune reconstitution and subsequent recovery of protective immunity are needed to safeguard bone marrow transplant recipients suffering from cancer against infectious agents and residual cancer cells that escaped chemotherapy (41, 42). The results show that A2-CAR Tregs can delay GvHD not only when the PBMCs themselves express HLA-A2, but also when HLA-A2 is expressed by the recipients and not by the PBMCs.

One potential benefit of eliminating endogenous TCR expression is to more precisely control Treg specificity, especially in the context of universal CAR Tregs for off-the-shelf use. Creating and banking such universal CAR Tregs may circumvent the challenge of expanding Tregs from immunosuppressed transplant recipients and the long production time that precludes their use in acute conditions. In this vein, it is shown in this study that one can simultaneously ablate HLA-A2 and TCR expression at high efficiency in human Tregs, while maintaining stable FOXP3 and HELIOS expression, as well as antigen-specific suppressive function in vitro and in vivo. Recently, progress has been made towards the generation of universal human pluripotent stem cells, which portend potential inexhaustible sources of universally compatible cells, tissues, and organs for therapy (43, 44). These data support the feasibility of developing universal engineered Tregs with precisely controlled specificity while evading host immune destruction to induce immune tolerance. Future experiments aimed at further characterizing the suppressive potency and longevity of CAR Tregs in vivo will shed additional light on the efficiency, safety, and feasibility of this strategy for Treg-based cell therapy.

In conclusion, it is demonstrated that it is feasible to engineer a grafted CAR directly into the TRAC locus of human Tregs. This strategy is highly efficient, does not cause Treg destabilization, and allowed for the generation of Tregs with CAR-restricted specificity that delayed GvHD in a target antigen-dependent manner. This strategy can be applied for precision engineering of therapeutic Tregs.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Materials and Methods for Examples 5-7

Animals: C57BL/6J mice (Jackson, stock number 000664), BALB/cJ mice (Jackson, stock number 000651), B6.Cg-Immp2l^{Tg(HLA-A/H2-D)2Enge}/J mice (Jackson, stock no. 004191, referred to as B6.A2 hereafter), B6.A2×BALB/c F1 mice, and TCR75 Tg mice [60, 61] were housed and bred under specific pathogen-free conditions at the University of California San Francisco Animal Barrier Facility. All mouse experiments were performed according to a UCSF Institutional Animal Care and Use Committee (IACUC) approved protocol.

Antibodies and reagents: The following antibodies were used for phenotyping and proliferation assays: anti-CD4 BUV496 (clone GK1.5, BD), anti-CD25 PE (clone PC61, Biolegend), anti-CD62L APC (clone MEL-14, BD), anti-CD69 PE-Cy7 (clone H1.2F3, BD), anti-Foxp3 AF488 (clone FJK-16s, Invitrogen), anti-myc tag AF488 or AF647 (clone 9B11, Cell signaling), anti-EGFRt PE or BV711 (clone AY13, Biolegend), anti-Thy1.1 PE-Cy7 (clone HIS51, eBioscience), anti-CD45.1 PE-Cy7 (clone A20, Biolegend), CellTrace Violet (Thermofischer), Live dead e780 (Thermofischer).

Cell Sorting andFlow Cytometry: CD4+ T cells were enriched from pooled LNs and spleens by negative selection using the EasySep™ Mouse CD4+ T Cell Isolation Kit (STEMCELL, Vancouver, Canada, Cat. #19852). The cells were then stained with anti-CD4-APC, anti-CD25-PE, and anti-CD62L-FITC, and the Tregs (CD4+CD25+CD62L^{hi}) and Tconv (CD4+CD25−CD62L^{hi}) were sorted on a FACS Aria II cytometer to >98% purity. Flow cytometric analyses were performed on a Fortessa or LSRII flow cytometer and FlowJo software.

CAR generation and retrovirus production: The construction of an anti-HLA-A2 single-chain fragment variable (scFv) was described previously (Muller Y D et al). Briefly, DNA sequences of the heavy and light chains of an anti-hHLA-A2 mAb was obtained from a human anti-HLA-A2 hybridoma clone SN607D8. The complementary determining regions (CDR) of the SN607D8 heavy and light chains were grafted onto the single-chain fragment variable (scFv) of the 4D5 human antibody scaffold of Herceptin. The chimeric antigen receptor (CAR) was then constructed by fusing a myc epitope tag, mouse CD8 hinge and transmembrane domains, and CD28 and CD3ζ intracellular domains. The resulting anti-HLA-A2-CAR (A2-CAR) was cloned into a murine stem cell virus (MSCV)-based retroviral vector in front of a truncated EGFR (EGFRt) separated by a P2A peptide. Viral particles were produced by using the Platinum-E (Plat-E) Retroviral Packaging Cell Line according to the manufacturer recommendations (Cell Biolabs, San Diego, CA).

In vitro expansion and transduction of T cells: Fluorescence-assisted cell sorting (FACS)-purified T cells were stimulated with anti-CD3/CD28 Dynabeads™ (Thermo Fisher Scientific (ThermoFisher), Waltham, MA, Cat. #11452D) supplemented with 2000 rhIL-2 IU/ml (Proleukin, Prometheus NDC 65483-116-07, San Diego, CA) for Tregs and 200 IU/ml rhIL-2 for the Tconv, in complete medium consisting of 10% heat-inactivated fetal bovine serum (Omega scientific, Tarzana, CA), nonessential amino acids, 0.5 mM sodium pyruvate, 5 mM HEPES, 1 mM glutaMax and 50 μM β-mercaptoethanol in DMEM high glucose medium. The cultures were monitored daily and maintained at 0.7-1.2×10⁶/ml by diluting with IL-2-supplemented complete medium for 8-12 days. On day 1 or 3 of the expansion culture, Tconv and Tregs, respectively, were transduced with retrovirus. On RetroNectin-precoated plates, retroviral particles were centrifugated for 2 h at 2000×g and spun again for 15 min at 2000×g after adding the T cells at 1×10⁶/ml. On day 8, T cells were sorted to purify transduced cells based on surface EGFRt-expression and rested overnight in complete medium containing no or 200 IU/ml IL-2 for the Tconv or Tregs, respectively, before using in subsequent experiments.

In vitro activation and suppression assay: A2-CAR+ T cells were co-cultivated with irradiated HLA-A2-expressing K562, parental HLA-A2-negative K562, splenocytes from B6 or B6.A2 mice for 2-3 days and activation markers or proliferation assessed by CTV-dilution were measured using flow cytometry. Tconv were maintained in medium without IL-2 and Tregs were maintained in 200 IU/ml IL-2 for activation assays and 2000 IU/ml IL-2 to assess proliferation. For the in vitro suppression assay, 25,000 CTV-labelled B6.TCR75 Tg+CD4+ responder cells were cultured with 175,000 irradiated splenocytes from B6.A2×BALB/c F1 or B6×BALB/c F1 and anti-CD3 and different ratios of polyclonal or A2.CAR+ Tregs in complete medium without IL-2. On day 4, cells were harvested, stained and analyzed using flow.

Heterotopic heart transplantation and Treg administration: Heterotopic, intra-abdominal heart transplantation was performed by transplanting heart from B6.A2 (single A2 mismatch) or B6.A2×BALB/c F1 (haplo mismatch) or B6 (syngeneic) mice into B6 mice as previously described [62]. Polyclonal or A2-CAR+ Tregs were administered intravenously one or two days after heart transplantation for the A2 F1 and single A2 mismatch model, respectively. Rapamycin treated mice received a therapeutic regimen of 1 mg/kg/day i.p. rapamycin from days 0 to 9 with or without an A2-CAR+ Treg i.v. injection on day 7 post heart transplantation as described previously [63]. Transplant survival was assessed by transabdominal palpation and graft function was scored on scale of 0 to 4 (0=non-beating, rejected graft; 1=contraction barely palpable, muscle fibrillation only; 2=obvious decrease in contraction strength but both ventricles still contracting in a coordinated manner; 3=strong, coordinated contraction of both ventricles but with noticeable decrease in strength or rate; 4=normal amplitude and frequency). Palpation scores of 0 and 1 were considered as rejection, which was confirmed histologically.

### Example 5. Generation of Murine Anti-HLA-A2-CAR Tconvs and Tregs

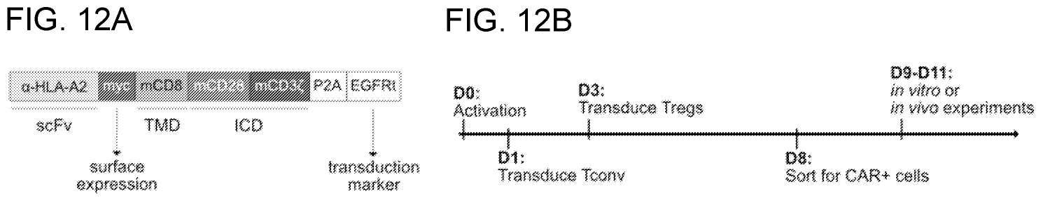

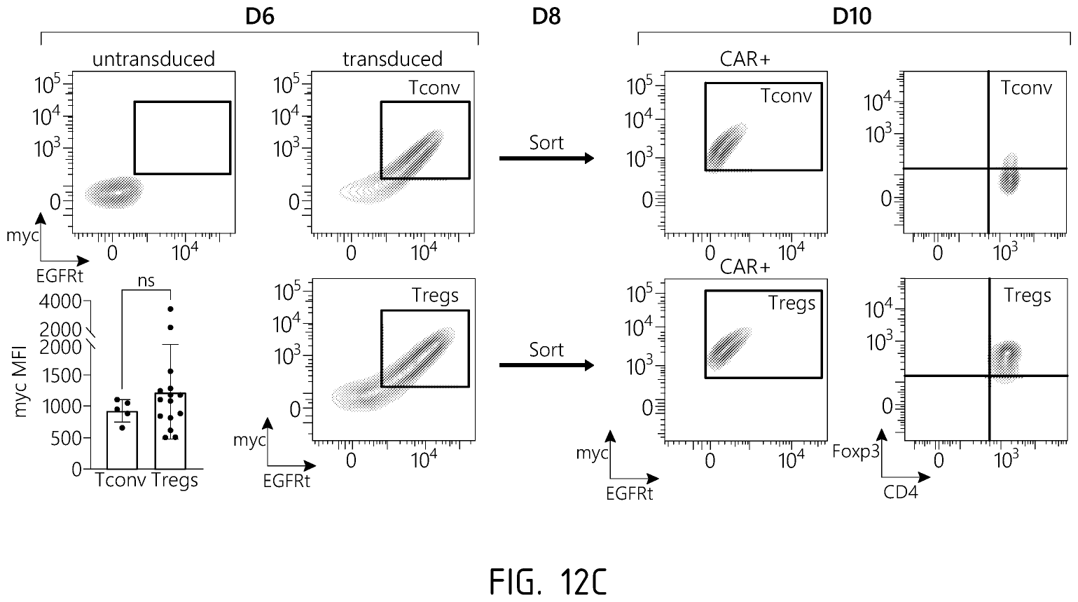

A murine anti-HLA-A2 CAR was designed by fusing an scFv recognizing human HLA-A2 to the mouse CD8 transmembrane domain, followed by a tandem arrangement of mouse CD28 and mouse CD3ζ intracellular domains (FIG. 12A). The scFv was generated by grafting the CDR of an anti-HLA-A2 antibody onto the framework of the Herceptin scFv (Muller Y D et al). A myc tag was used to detect surface expression and EGFRt was used as a transduction marker. Tconvs and Tregs were FACS purified from lymph nodes and spleens of B6.Thy1.1 mice and activated with anti-CD3 and anti-CD28-conjugated (aCD3/28) beads prior to retroviral transduction with the CAR construct on day 1 or 3 after initial activation, respectively. On day 8, expanded Tconvs and Tregs were sorted for CAR-positive cells based on EGFRt-expression (FIG. 12B). The transduction efficiency determined by myc- and EGFRt-expression as well as the myc MFI (mean fluorescent intensity) was comparable in Tconvs and Tregs (transduction efficiency: 67.0±2.0%, n=5 and 56.96±3.48%, n=15; p=0.14, data not shown; myc MFI: 929.0±158.61, n=5 and 1238±732.21, n=15; p=0.3867, FIG. 12C). After activation, transduction, and sort for CAR-positive cells, the Tregs remained Foxp3-positive.

### Example 6. A2-CAR Treg Activation and Suppression Function in Response to HLA-A2 Antigen

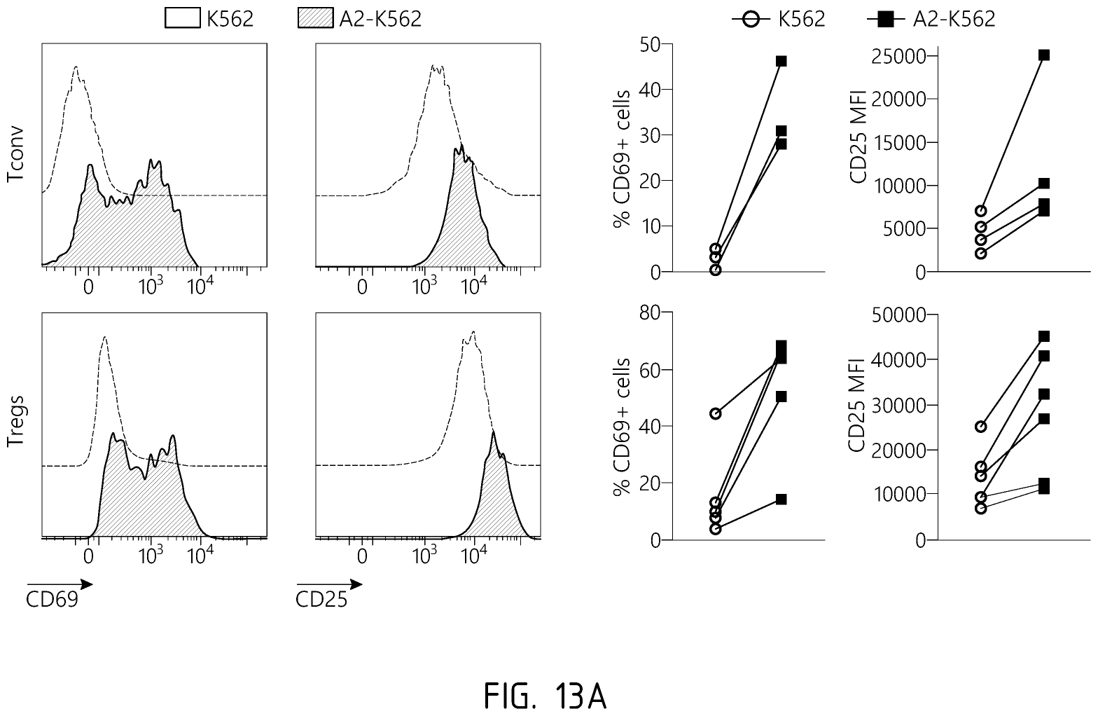

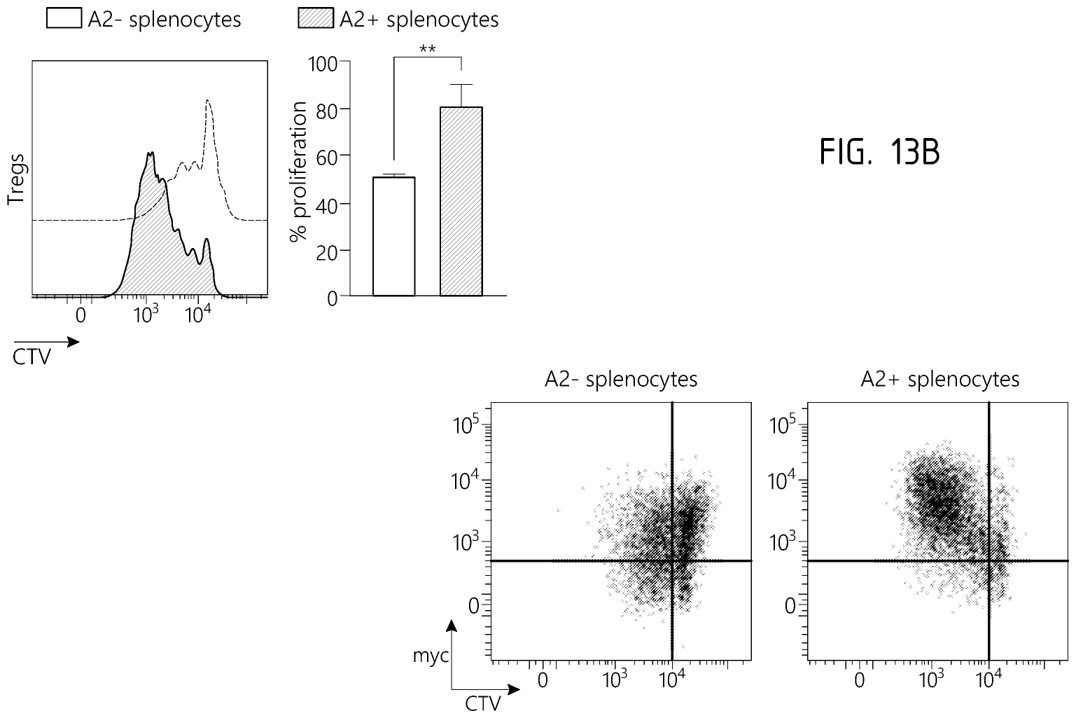

To validate the HLA-A2-reactivity of A2-CAR T cells, A2-CAR-expressing Tconvs or Tregs were co-incubated with parental K562 or K562 cells that have been genetically modified to express HLA-A2. Tconvs and Tregs both upregulated T-cell activation markers including CD25 and CD69 when co-incubated with A2-expressing K562 cells but not parental K562 cells (FIG. 13A). Similarly, irradiated splenocytes from B6.A2 mice induced a significantly higher rate of proliferation in A2-CAR Tregs than that induced by splenocytes from B6 mice (FIG. 2B).

Next, the ability of A2-CAR Tregs to suppress the proliferation of CD4+ from B6.TCR75 transgenic mice was evaluated in vitro. TCR75 TCR is specific for a H2-K^{d}-derived peptide presented by H2-Ia^{b} [61], and thus recognizes BALB/c-derived alloantigen presented by B6 antigen presenting cells. In the absence of Tregs, CTV-labeled TCR75 transgenic T cells proliferated robustly in response to stimulation with irradiated splenocytes from B6.A2×BALB/c F1 (FIG. 13C). Addition of A2-CAR Tregs strongly inhibited the proliferation of TCR75 T cells, exhibiting 8 to 16-fold greater suppressive activity than polyclonal Tregs (p<0.0001, FIG. 13C). This difference in suppressive activities is most likely due to the relatively low frequency of naturally A2-reactive clones within the untransduced Treg population. Importantly, Treg stimulation via anti-CD3 elicited comparable suppressive capacity between A2-CAR Tregs and polyclonal, untransduced Tregs, indicating that A2-CAR expression does not alter TCR-mediated mechanisms of suppression (p=0.6506, FIG. 13C). Together, these results demonstrate that the A2-CAR triggers Treg activation and suppressive function specifically in response to HLA-A2 antigen.

### Example 7. A2-CAR Tregs Prolong Graft Survival in Vascularized Transplantation Models

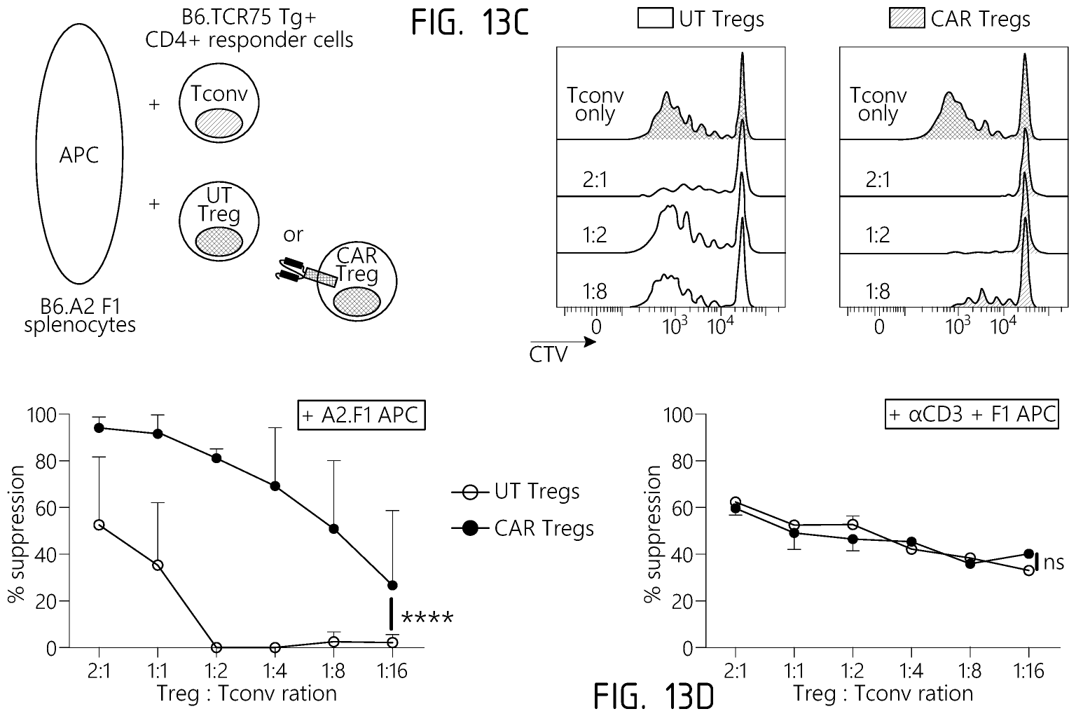

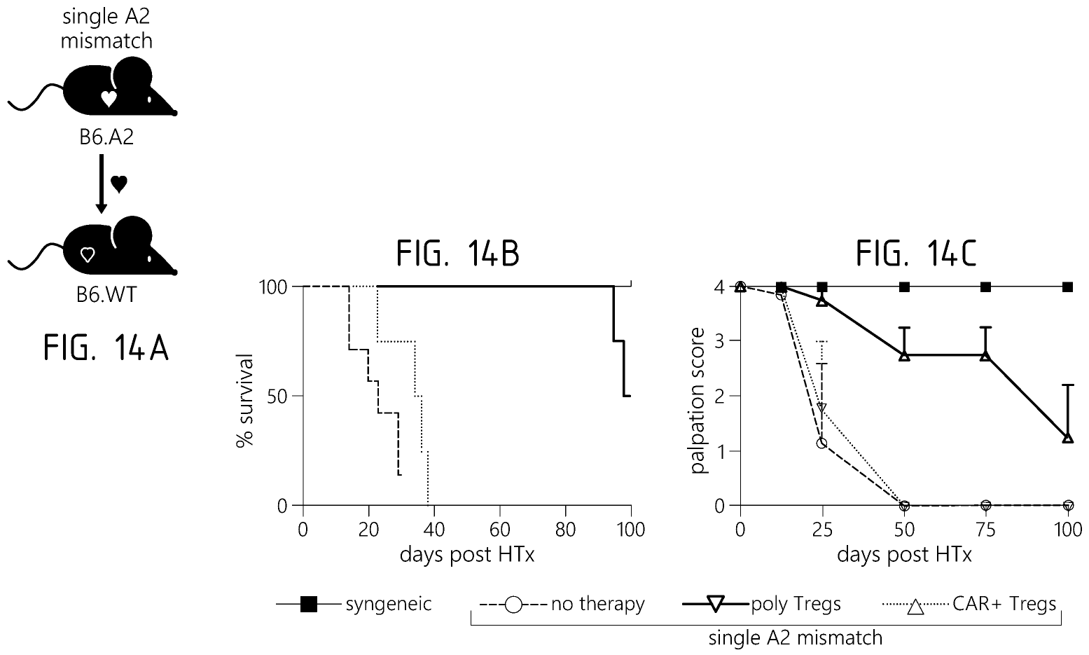

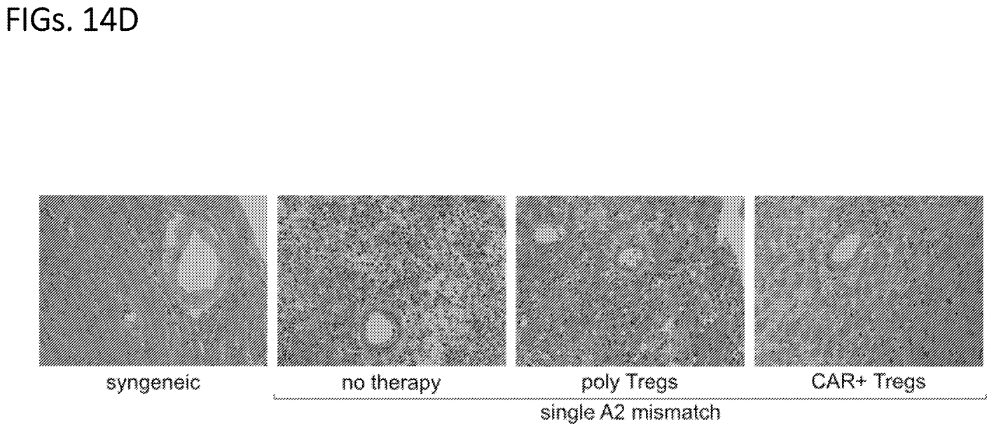

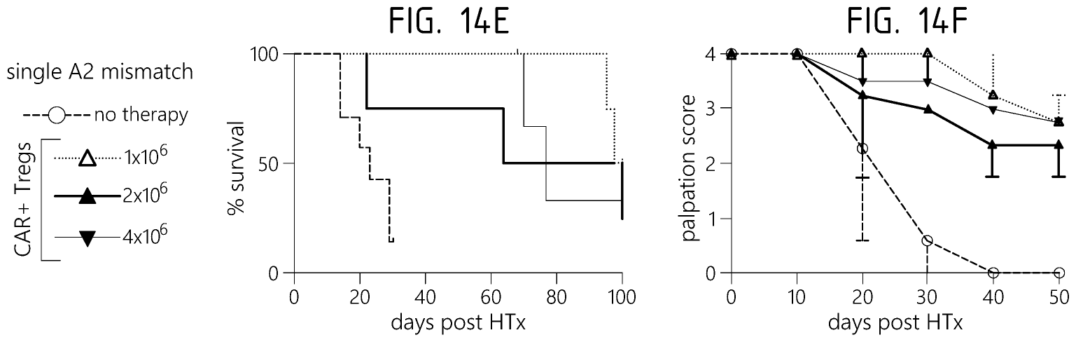

To further assess the in vivo function of A2-CAR Tregs, a single A2-mismatch transplantation model was developed in which hearts from B6.A2 mouse were transplanted heterotopically into B6 recipients (FIG. 14A). The heart transplant in this single A2-mismatch model had a median survival of 23 days (n=7). Injecting 1×10⁶ polyclonal Tregs on day 2 after transplantation led to a slight prolongation of graft survival (median survival 35 days, n=4, p=0.0754, FIG. 14B). By comparison, 1×10⁶ A2-CAR-Tregs significantly prolonged the median graft survival to 99 days (n=4, p=0.0144, FIG. 14B). Despite the significant improvement in graft survival, alloimmune responses to the graft were not completely controlled, reflected by the progressive decline of the palpation score over time (FIG. 14C). Histological analysis of the grafts that survived to day 100 showed lymphocytic infiltrates in the graft tissue signs of cell-mediated rejection (FIG. 14D).

Since 1×10⁶ A2-CAR-Tregs did not completely prevent graft rejection, it was determined if higher doses A2-CAR-Tregs could provide better graft protection. Injection of either 2×10⁶ or 4×10⁶ A2-CAR-Tregs did not lead to better prolongation of graft function (FIG. 14E) or better graft function by palpitation score (FIG. 14F). There was no significant difference between the three tested A2-CAR Treg-doses (p=0.6617).

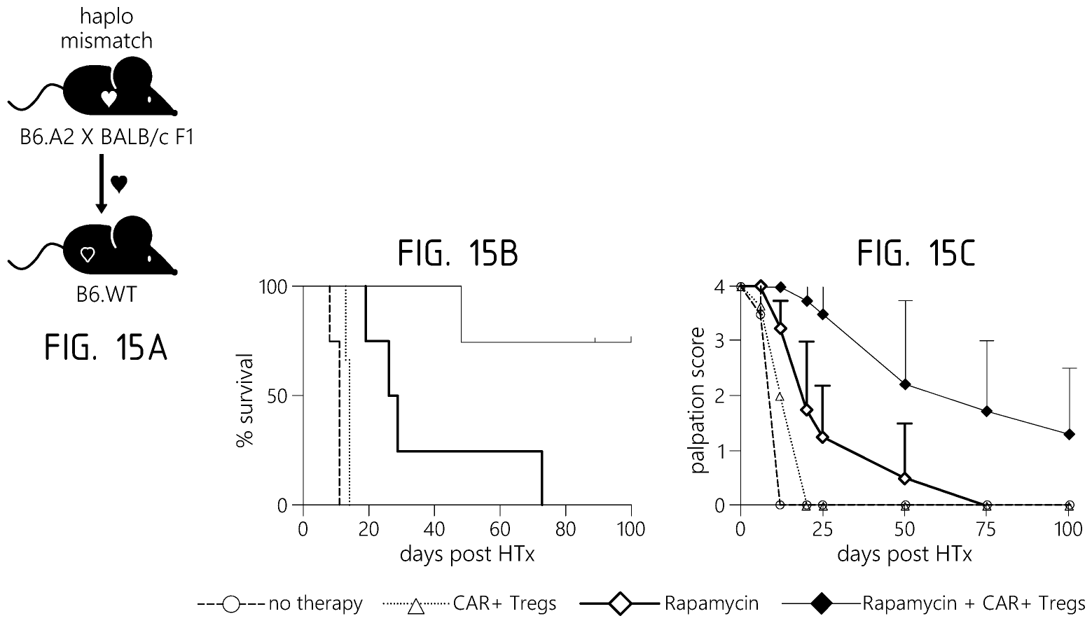

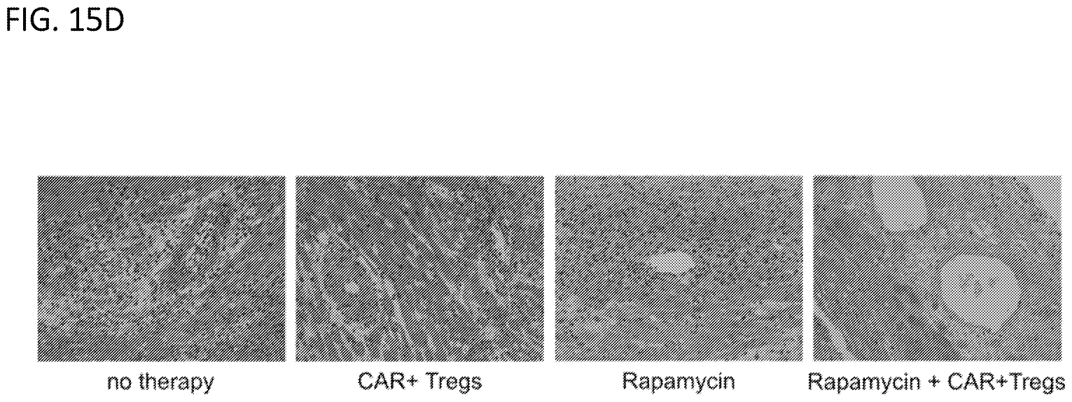

To determine if A2-CAR Tregs could provide protection to more immunogenic grafts, a haplo-mismatched model was used by transplanting hearts from B6.A2×BALB/c F1 mice heterotopically into B6 recipients (FIG. 15A). The median survival of the allografts was only 11 days (n=4). Injecting 1×10⁶ A2-CAR Tregs on day 1 after transplantation significantly prolonged graft survival, albeit by only 3 days (median survival 14 days, n=4, p=0.0207, FIG. 15B). A previous study reported a synergistic effect on mouse heart allograft survival by combining alloantigen specific Tregs and rapamycin [63]. The combined treatment of rapamycin and A2-CAR Tregs was thus investigated. It was observed that treatment of Rapamycin for 9 days postoperatively led to a significant prolongation of graft survival (median survival 27.5 days, n=4, p=0.01, FIG. 15B) when compared to the untreated group. Adding 1×10⁶ A2-CAR Tregs on d7 of the rapamycin regimen significantly prolonged graft survival (n=4, p=⁰0.0266 compared to rapamycin-only treatment group and p=0.01 to the untreated group, FIG. 15B). However, similar to the single A2-mismatch model, A2-CAR+ Tregs in combination with rapamycin were not able to completely prevent graft rejection, observed in the reduction of palpation score (FIG. 15C) as well as lymphocytic infiltrates in the graft tissue (FIG. 15D).

### DISCUSSION

To date, studies characterizing the impact of CAR Tregs in transplantation have primarily focused on the prevention of skin graft rejection or systemic GvHD in humanized mouse models. Despite demonstrating the therapeutic potential of antigen-specific CAR Tregs within these contexts [54-58], the lack of full immune-cell reconstitution and graft vascularization greatly limit the applicability of the findings to a clinical setting. While a recent study was able to show prolongation of skin graft survival in immunocompetent mice [59], the evaluation of CAR-Treg function within a vascularized transplantation model has yet to be reported. In this study, it is shown that the infusion of only 1×10⁶ CAR+ Tregs in a vascularized, heterotopic heart transplantation model was able to significantly prolong allograft survival in immunocompetent mice.

Although a significant prolongation of allograft survival was observed, the grafts exhibited increased lymphocyte infiltration and correspondingly reduced graft function over time, indicating that a single dose of 1×10⁶ CAR Tregs was insufficient to induce complete tolerance. Similarly, in a skin transplantation model in immunocompetent mice, Sicard et al. reported prolongation but eventual rejection of the graft with the same dose of CAR Tregs [59]. As the safety of CAR Treg therapy is paramount for translation into the clinical setting, we increased the initial dose to 2×10⁶ or 4×10⁶ CAR Tregs and closely monitored the graft function by palpation score. One of the mechanisms by which Tregs suppress is mediated by direct cytotoxicity to antigen-presenting cells (APCs) via delivery of perforin and granzyme B [64]. As Tregs naturally interact with APCs expressing class II MHC molecules, direct cytotoxicity reduces the availability of presented antigen, and thus restricts effector T-cell activation and subsequent graft rejection. However, CAR-mediated targeting of Tregs to parenchymal and vascular endothelial cells expressing class I MHC molecules avoids direct toxicity that may otherwise result in the loss of graft function. Previous in vitro experiments have shown that the short-term culture of A2-CAR Tregs with HLA-A2-positive K562 cells [54] or HLA-A2-positive primary epithelial layers [58] did not lead to obvious cell death. Similarly, in the present in vivo model, doubling or quadrupling the effective dose of A2-CAR Tregs did not show toxic effects on the graft function after injection. However, since increased single-dose administrations also could not prevent graft infiltration and rejection over time, multi-dose administration of CAR Tregs should be evaluated in future studies for potential enhancement of allograft protection.

For the transplant setting in humans, typically more than one HLA is mismatched, raising the question of whether CAR Tregs targeting one of the mismatched HLAs would be sufficient for preventing allograft rejection. All studies thus far have focused on single A2-mismatch models. To evaluate A2-CAR function in a more clinically relevant setting, the heterotopic heart transplantation model was used in which hearts from B6.A2×BALB/c F1 donors are transplanted into B6 mice. In this setting, the donor tissue expresses multiple MHC mismatches that are not targeted by the A2-CAR. While the combination of rapamycin administration and infusion of 1×10⁶ A2-CAR Tregs significantly prolonged graft survival, the treatment was ultimately insufficient for inducing tolerance and preventing allograft rejection. Previously, it has been shown that the injection of alloantigen-specific Tregs in combination with rapamycin treatment can lead to long-term graft survival of heart transplants for over 150 days post transplantation [63]. In this setting, polyclonal Tregs were co-incubated with BALB/c-derived dendritic cells to enrich for the alloantigen-specific population that can target the BALB/c heart allograft. Alloantigen-specific Tregs are activated upon recognition of MHC class II, mostly through the indirect or semi-direct pathway of allorecognition as donor APCs are short-lived [65]. In comparison, since A2-CAR Tregs recognize the A2-expression either on the allograft via the direct pathway or in the lymph node via the semidirect pathway, the main site of Treg-mediated suppression is most likely localized to the graft itself. However, the promotion of long-term graft survival through Tregs is mediated by their indirect allospecificity [49], thus potentially explaining why A2-CAR Tregs promote graft survival but do not prevent rejection. TCR transgenic direct Tregs can induce graft tolerance potentially by inducing quiescent state to allow endogenous indirect Tregs to expand [51]. It would be of interest to directly compare the efficacy of MHC class I- and MHC class II-targeting CAR Tregs with direct and indirect allospecificity. However, a strategy to synthetically transfer indirect allospecificity remains to be developed.

In summary, it is shown that the injection of A2-CAR Tregs in the heterotopic heart transplantation model significantly prolongs graft survival in the single A2-mismatch as well as a haplo-mismatch model. Although further studies are required to characterize the site(s) of suppressive activity and further optimize treatment strategies to promote indefinite graft survival, this study offers the first demonstration of CAR-Treg-mediated long-term graft survival in a fully immunocompetent vascularized graft model.

### REFERENCES

1. Esensten J H, Muller Y D, Bluestone J A, Tang Q. Regulatory T cell therapy for autoimmune and autoinflammatory diseases: the next frontier. J Allergy Clin Immunol. (2018) 142:1710-18. doi: 10.1016/j.jaci.2018.10.015
2. Tang Q, Vincenti F. Transplant trials with Tregs: perils and promises. J. Clin. Invest. (2017) 127: 2505-12. doi: 10.1172/JC190598
3. Chandran S, Tang Q, Sarwal M, Laszik Z G, Putnam A L, Lee K, Leung J, Nguyen V, Sigdel T, Tavares E C, Yang J Y C, Hellerstein M, Fitch M, Bluestone J A, Vincenti F. Polyclonal regulatory T cell therapy for control of inflammation in kidney transplants. Am. J Transplant. (2017) 17:2945-54. doi: 10.1111/ajt.14415
4. Mathew J M, H-Voss J, LeFever A, Konieczna I, Stratton C, He J, Huang X, Gallon L, Skaro A, Ansari M J, Leventhal J R. A Phase I clinical trial with ex vivo expanded recipient regulatory T cells in living donor kidney transplants. Sci. Rep. (2018) 8:7428. doi: 10.1038/s41598-018-25574-7
5. Sawitzki B, Harden P N, Reinke P, Moreau A, Hutchinson J A, Game D S, Tang Q, Guinan E C, Battaglia M, Burlingham W J, Roberts I S D, Streitz M, Josien R, Böger C A, Scottà C, Markmann J F, Hester J L, Juerchott K, Braudeau C, James B, Contreras-Ruiz L, van der Net J B, Bergler T, Caldara R, Petchey W, Edinger M, Dupas N, Kapinsky M, Mutzbauer I, Otto N M, Öllinger R, Hernandez-Fuentes M P, Issa F, Ahrens N, Meyenberg C, Karitzky S, Kunzendorf U, Knechtle S J, Grinyó J, Morris P J, Brent L, Bushell A, Turka L A, Bluestone J A, Lechler R I, Schlitt H J, Cuturi M C, Schlickeiser S, Friend P J, Miloud T, Scheffold A, Secchi A, Crisalli K, Kang S M, Hilton R, Banas B, Blancho G, Volk H D, Lombardi G, Wood K J, Geissler E K. Regulatory cell therapy in kidney transplantation (The ONE Study): a harmonised design and analysis of seven non-randomised, single-arm, phase 1/2A trials. Lancet. (2020) 395:1627-39. doi: 10.1016/S0140-6736(20)30167-7
6. Lee K, Nguyen V, Lee K M, Kang S M, Tang Q. Attenuation of donor-reactive T cells allows effective control of allograft rejection using regulatory T cell therapy. Am. J. Transplant. (2014) 14:27-38. doi: 10.1111/ajt.12509
7. Wagner, J. C., Tang, Q. CAR-Tregs as a strategy for inducing graft tolerance. Curr. Transplant. Rep. (2020) 7:205-14. doi: 10.1007/s40472-020-00285-z
8. Sicard A, Levings M K, Scott D W. Engineering therapeutic T cells to suppress alloimmune responses using TCRs, CARs, or BARs. Am. J Transplant. (2018) 18:1305-11. doi: 10.1111/ajt.14747
9. Ferreira L M R, Muller Y D, Bluestone J A, Tang Q. Next-generation regulatory T cell therapy. Nat. Rev. Drug Discov. (2019) 18:749-69. doi: 10.1038/s41573-019-0041-4
10. MacDonald K G, Hoeppli R E, Huang Q, Gillies J, Luciani D S, Orban P C, Broady R, Levings M K. Alloantigen-specific regulatory T cells generated with a chimeric antigen receptor. J. Clin. Invest. (2016) 126:1413-24. doi: 10.1172/JC182771
11. Boardman D A, Philippeos C, Fruhwirth G O, Ibrahim M A, Hannen R F, Cooper D, Marelli-Berg F M, Watt F M, Lechler R I, Maher J, Smyth L A, Lombardi G. Expression of a chimeric antigen receptor specific for donor HLA class I enhances the potency of human regulatory T cells in preventing human skin transplant rejection. Am. J. Transplant. (2017) 17:931-43. doi: 10.1111/ajt.14185
12. Noyan F, Zimmermann K, Hardtke-Wolenski M, Knoefel A, Schulde E, Geffers R, Hust M, Huehn J, Galla M, Morgan M, Jokuszies A, Manns M P, Jaeckel E. Prevention of allograft rejection by use of regulatory T cells with an MHC-specific chimeric antigen receptor. Am. J. Transplant. (2017) 17:917-30. doi: 10.1111/ajt.14175
13. Eyquem J, Mansilla-Soto J, Giavridis T, van der Stegen S J, Hamieh M, Cunanan K M, Odak A, Gönen M, Sadelain M. Targeting a CAR to the TRAC locus with CRISPR/Cas9 enhances tumour rejection. Nature. (2017) 543:113-17. doi: 10.1038/nature21405
14. Muller Y D, Nguyen D P, Ferreira L M R, Ho P, Raffin C, Valencia R V B, Congrave-Wilson Z, Roth T, Eyquem J, Van Gool F, Marson A, Perez L, Wells J A., Bluestone J A., Tang Q. The CD28-transmembrane domain mediates chimeric antigen receptor heterodimerization with CD28. Front. Immunol. (2021) 12:639818. doi: 10.3389/fimmu.2021.639818
15. Putnam A L, Brusko T M, Lee M R, Liu W, Szot G L, Ghosh T, Atkinson M A, Bluestone J A. Expansion of human regulatory T-cells from patients with type 1 diabetes. Diabetes. (2009) 58:652-62. doi: 10.2337/db08-1168
16. Mulder A, Eijsink C, Kardol M J, Franke-van Dijk M E, van der Burg S H, Kester M, Doxiadis I I, Claas F H. Identification, isolation, and culture of HLA-A2-specific B lymphocytes using MHC class I tetramers. J. Immunol. (2003) 171:6599-603. doi: 10.4049/jimmunol.171.12.6599
17. Sheets M D, Amersdorfer P, Finnern R, Sargent P, Lindquist E, Schier R, Hemingsen G, Wong C, Gerhart J C, Marks J D. Efficient construction of a large nonimmune phage antibody library: the production of high-affinity human single-chain antibodies to protein antigens. Proc. Natl. Acad. Sci. U.S.A. (1998) 95:6157-62. doi: 10.1073/pnas.95.11.6157
18. Battle R, Poole K, Haywood-Small S, Clark B, Woodroofe M N. Molecular characterisation of the monocytic cell line THP-1 demonstrates a discrepancy with the documented HLA type. Int. J. Cancer. (2013) 132:246-7. doi: 10.1002/ijc.27661
19. Adams S, Robbins F M, Chen D, Wagage D, Holbeck S L, Morse H C, Stroncek D, Marincola F M. HLA class I and II genotype of the NCI-60 cell lines. J. Transl. Med. (2005) 3:11. doi:10.1186/1479-5876-3-11
20. Waterhouse A M, Procter J B, Martin D M, Clamp M, Barton G J. Jalview Version 2—a multiple sequence alignment editor and analysis workbench. Bioinformatics. (2009) 25:1189-91. doi:10.1093/bioinformatics/btp033
21. Kunik V, Ashkenazi S, Ofran Y. Paratome: an online tool for systematic identification of 527antigen-binding regions in antibodies based on sequence or structure. Nucleic Acids Res. (2012)40:W521-4. doi: 10.1093/nar/gks480
22. Yousefzadeh M J, Wyatt D W, Takata K, Mu Y, Hensley S C, Tomida J, Bylund G O, Doublie S, Johansson E, Ramsden D A, McBride K M, Wood R D. Mechanism of suppression of chromosomal instability by DNA polymerase POLQ. PLoS Genet. (2014) 10:e1004654. doi:10.1371/journal.pgen.1004654
23. Ho P, Ede C, Chen Y Y. Modularly constructed synthetic granzyme B molecule enables interrogation of intracellular proteases for targeted cytotoxicity. ACS Synth. Biol. (2017) 6:1484-95. doi: 10.1021/acssynbio.6b00392
24. Xiao W, Chirmule N, Berta S C, McCullough B, Gao G, Wilson J M. Gene therapy vectors based on adeno-associated virus type 1. J. Virol. (1999) 73:3994-4003. doi: 10.1128/JVI.73.5.3994-4003.1999
25. Grieger J C, Choi V W, Jude Samulski R. Production and characterization of adeno-associated viral vectors. Nat. Protoc. (2006) 1:1412-28. doi: 10. 1038/nprot.2006.207
26. Dawson N A, Lamarche C, Hoeppli R E, Bergqvist P, Fung V C, McIver E, Huang Q, Gillies J, Speck M, Orban P C, Bush J W, Mojibian M, Levings M K. Systematic testing and specificity mapping of alloantigen-specific chimeric antigen receptors in regulatory T cells. JCI Insight. (2019) 4:e123672. doi: 10.1172/jci.insight.123672
27. Roth T L, Puig-Saus C, Yu R, Shifrut E, Carnevale J, Li P J, Hiatt J, Saco J, Krystofinski P, Li H, Tobin V, Nguyen D N, Lee M R, Putnam A L, Ferris A L, Chen J W, Schickel J N, Pellerin L, Carmody D, Alkorta-Aranburu G, Del Gaudio D, Matsumoto H, Morell M, Mao Y, Cho M, Quadros R M, Gurumurthy C B, Smith B, Haugwitz M, Hughes S H, Weissman J S, Schumann K, Esensten J H, May A P, Ashworth A, Kupfer G M, Greeley S A W, Bacchetta R, Meffre E, Roncarolo M G, Romberg N, Herold K C, Ribas A, Leonetti M D, Marson A. Reprogramming human T cell function and specificity with non-viral genome targeting. Nature. (2018) 559:405-9. doi: 10.1038/s41586-018-0326-5
28. Szot G L, Koudria P, Bluestone J A. Transplantation of pancreatic islets into the kidney capsule of diabetic mice. J. Vis. Exp. (2007) (9):404. doi: 10.3791/404
29. Szot G L, Lee M R, Tavakol M M, Lang J, Dekovic F, Kerlan R K, Stock P G, Posselt A M. Successful clinical islet isolation using a GMP-manufactured collagenase and neutral protease. Transplantation. (2009) 88:753-6. doi: 10.1097/TP.0b013e3181b443ae
30. Zhao Y, Wang Q J, Yang S, Kochenderfer J N, Zheng Z, Zhong X, Sadelain M, Eshhar Z, Rosenberg S A, Morgan R A. A herceptin-based chimeric antigen receptor with modified signaling domains leads to enhanced survival of transduced T lymphocytes and antitumor activity. J Immunol. (2009) 183:5563-74. doi: 10.4049/jimmunol.0900447
31. Leem J, Dunbar J, Georges G, Shi J, Deane C M. ABodyBuilder: Automated antibody structure prediction with data-driven accuracy estimation. MAbs. (2016) 8:1259-68. doi: 10.1080/19420862.2016.1205773
32. Mulder A, Kardol M, Regan J, Buelow R, Claas F. Reactivity of twenty-two cytotoxic human monoclonal HLA antibodies towards soluble HLA class I in an enzyme-linked immunosorbent assay (PRA-STAT). Hum. Immunol. (1997) 56:106-13. doi: 10.1016/s0198-8859(97)00146-8
33. DeWolf S, Grinshpun B, Savage T, Lau S P, Obradovic A, Shonts B, Yang S, Morris H, Zuber J, Winchester R, Sykes M, Shen Y. Quantifying size and diversity of the human T cell alloresponse. JCI Insight. (2018) 3:e121256. doi: 10.1172/jci.insight.121256
34. Gress R E, Nathenson S G, Lucas P J. Fine specificity of xenogeneic antigen recognition by human T cells. Transplantation. (1989) 48:93-8. doi: 10.1097/00007890-198907000-00022
35. Bluestone J A, Buckner J H, Fitch M, Gitelman S E, Gupta S, Hellerstein M K, Herold K C, Lares A, Lee M R, Li K, Liu W, Long S A, Masiello L M, Nguyen V, Putnam A L, Rieck M, Sayre P H, Tang Q. Type 1 diabetes immunotherapy using polyclonal regulatory T cells. Sci Transl Med. (2015) 7:315ra189. doi: 10.1126/scitranslmed.aad4134
36. Watkins N A, Brown C, Hurd C, Navarrete C, Ouwehand W H. The isolation and characterisation of human monoclonal HLA-A2 antibodies from an immune V gene phage display library. Tissue Antigens. (2000) 55:219-28. doi: 10.1034/j.1399-0039.2000.550305.x
37. Rijkers M, Schmidt D, Lu N, Kramer C S M, Heidt S, Mulder A, Porcelijn L, Claas F H J, Leebeek F W G, Jansen A J G, Jongerius I, Zeerleder S S, Vidarsson G, Voorberg J, de Haas M. Anti-HLA antibodies with complementary and synergistic interaction geometries promote classical complement activation on platelets. Haematologica. (2019) 104:403-16. doi:10.3324/haematol.2018.201665
38. El-Awar N, Jucaud V, Nguyen A. HLA epitopes: The targets of monoclonal and alloantibodies defined. J Immunol Res. (2017) 2017:3406230. doi: 10.1155/2017/3406230
39. Wyss L, Stadinski B D, King C G, Schallenberg S, McCarthy N I, Lee J Y, Kretschmer K, Terracciano L M, Anderson G, Surh C D, Huseby E S, Palmer E. Affinity for self antigen selects Treg cells with distinct functional properties. Nat. Immunol. (2016) 17:1093-101. doi: 10.1038/ni.3522
40. Levine A G, Arvey A, Jin W, Rudensky A Y. Continuous requirement for the TCR in regulatory T cell function. Nat. Immunol. (2014) 15:1070-8. doi: 10.1038/ni.3004
41. Edinger M, Hoffmann P, Ermann J, Drago K, Fathman C G, Strober S, Negrin R S. CD4+CD25+ regulatory T cells preserve graft-versus-tumor activity while inhibiting graft-versus-host disease after bone marrow transplantation. Nat. Med. (2003) 9:1144-50. doi: 10.1038/nm915
42. Shlomchik W D. Graft-versus-host disease. Nat. Rev. Immunol. (2007) 7:340-52. doi:10.1038/nri2000
43. Han X, Wang M, Duan S, Franco P J, Kenty J H, Hedrick P, Xia Y, Allen A, Ferreira L M R, Strominger J L, Melton D A, Meissner T B, Cowan C A. Generation of hypoimmunogenic human pluripotent stem cells. Proc. Natl. Acad. Sci. U.S.A. (2019) 116:10441-6. doi: 10.1073/pnas.1902566116
44. Deuse T, Hu X, Gravina A, Wang D, Tediashvili G, De C, Thayer W O, Wahl A, Garcia J V, Reichenspurner H, Davis M M, Lanier L L, Schrepfer S. Hypoimmunogenic derivatives of induced pluripotent stem cells evade immune rejection in fully immunocompetent allogeneic recipients. Nat. Biotechnol. (2019) 37:252-8. doi: 10.1038/s41587-019-0016-3
45. Vlachakis D, Koumandou V L, Kossida S. A holistic evolutionary and structural study of flaviviridae provides insights into the function and inhibition of HCV helicase. Peer J. (2013) 1:e74. doi: 10.7717/peerj.74
46. El-Awar N, Lee J H, Tarsitani C, Terasaki P I. HLA class I epitopes: recognition of binding sites by mAbs or eluted alloantibody confirmed with single recombinant antigens. Hum. Immunol. (2007) 68:170-80. doi: 10.1016/j.humimm.2006.11.006
47. Tang Q, Bluestone J A (2013) Regulatory T-cell therapy in transplantation. Moving to the clinic. Cold Spring Harbor perspectives in medicine 3 (11).
48. Brennan T V, Tang Q, Liu F-C, Hoang V, Bi M et al. (2011) Requirements for prolongation of allograft survival with regulatory T cell infusion in lymphosufficient hosts. The Journal of surgical research 169 (1): e69-75.
49. Tsang J Y-S, Tanriver Y, Jiang S, Xue S-A, Ratnasothy K et al. (2008) Conferring indirect allospecificity on CD4+CD25+ Tregs by TCR gene transfer favors transplantation tolerance in mice. The Journal of clinical investigation 118 (11): 3619-3628.
50. Joffre O, Santolaria T, Calise D, A1 Saati T, Hudrisier D et al. (2008) Prevention of acute and chronic allograft rejection with CD4+CD25+Foxp3+ regulatory T lymphocytes. Nature medicine 14 (1): 88-92.
51. Lee K, Nguyen V, Lee K-M, Kang S-M, Tang Q (2014) Attenuation of donor-reactive T cells allows effective control of allograft rejection using regulatory T cell therapy. American journal of transplantation: official journal of the American Society of Transplantation and the American Society of Transplant Surgeons 14 (1): 27-38.
52. Sagoo P, Ali N, Garg G, Nestle F O, Lechler R I et al. (2011) Human regulatory T cells with alloantigen specificity are more potent inhibitors of alloimmune skin graft damage than polyclonal regulatory T cells. Science translational medicine 3 (83): 83ra42.
53. Samy E T, Parker L A, Sharp C P, Tung K S K (2005) Continuous control of autoimmune disease by antigen-dependent polyclonal CD4+CD25+ regulatory T cells in the regional lymph node. The Journal of experimental medicine 202 (6): 771-781.
54. MacDonald K G, Hoeppli R E, Huang Q, Gillies J, Luciani D S et al. (2016) Alloantigen-specific regulatory T cells generated with a chimeric antigen receptor. The Journal of clinical investigation 126 (4): 1413-1424.
55. Boardman D A, Philippeos C, Fruhwirth G O, Ibrahim M A A, Hannen R F et al. (2017) Expression of a Chimeric Antigen Receptor Specific for Donor HLA Class I Enhances the Potency of Human Regulatory T Cells in Preventing Human Skin Transplant Rejection. American journal of transplantation: official journal of the American Society of Transplantation and the American Society of Transplant Surgeons 17 (4): 931-943.
56. Noyan F, Zimmermann K, Hardtke-Wolenski M, Knoefel A, Schulde E et al. (2017) Prevention of Allograft Rejection by Use of Regulatory T Cells With an MHC-Specific Chimeric Antigen Receptor. American journal of transplantation: official journal of the American Society of Transplantation and the American Society of Transplant Surgeons 17 (4): 917-930.
57. Dawson N A, Lamarche C, Hoeppli R E, Bergqvist P, Fung V C et al. (2019) Systematic testing and specificity mapping of alloantigen-specific chimeric antigen receptors in regulatory T cells. JCI insight 4 (6).
58. Bezie S, Charreau B, Vimond N, Lasselin J, Gerard N et al. (2019) Human CD8+ Tregs expressing a MHC-specific CAR display enhanced suppression of human skin rejection and GVHD in NSG mice. Blood advances 3 (22): 3522-3538.
59. Sicard A, Lamarche C, Speck M, Wong M, Rosado-Sánchez I et al. (2020) Donor-specific chimeric antigen receptor Tregs limit rejection in naive but not sensitized allograft recipients. American journal of transplantation: official journal of the American Society of Transplantation and the American Society of Transplant Surgeons 20 (6): 1562-1573.
60. Brennan T V, Jaigirdar A, Hoang V, Hayden T, Liu F-C et al. (2009) Preferential priming of alloreactive T cells with indirect reactivity. American journal of transplantation: official journal of the American Society of Transplantation and the American Society of Transplant Surgeons 9 (4): 709-718.
61. Honjo K, Xu Xy, Bucy R P (2004) CD4+ T-cell receptor transgenic T cells alone can reject vascularized heart transplants through the indirect pathway of alloantigen recognition. Transplantation 77 (3): 452-455.
62. Plenter R J, Grazia T J (2014) Murine heterotopic heart transplant technique. Journal of visualized experiments: JoVE (89).
63. Raimondi G, Sumpter T L, Matta B M, Pillai M, Corbitt N et al. (2010) Mammalian target of rapamycin inhibition and alloantigen-specific regulatory T cells synergize to promote long-term graft survival in immunocompetent recipients. Journal of immunology (Baltimore, Md.: 1950) 184 (2): 624-636.
64. Schmidt A, Oberle N, Krammer P H (2012) Molecular Mechanisms of Treg-Mediated T Cell Suppression. Frontiers in immunology 3.
65. Wagner J C, Tang Q (2020) CAR-Tregs as a Strategy for Inducing Graft Tolerance. Current transplantation reports 7 (3): 205-214.
66. Daga et al (2018) Direct Quantitative Measurement of the Kinetics of HLA-specific Antibody Interactions with Isolated HLA Proteins. Human Immunology 79 (2):122-128.

1. A chimeric-antigen-receptor regulatory T cell (CAR-Treg) comprising:
an exogenous nucleic acid encoding a chimeric antigen receptor (CAR) comprising the complementarity determining regions (CDRs) of a human anti-HLA-A2 antibody grafted onto an antibody scaffold;
a hinge domain;
a transmembrane domain; and
an intracellular domain, wherein the CAR-Treg expresses the CAR on the cell surface.

2. The CAR-Treg of claim 1, wherein the antibody scaffold is human trastuzumab.

3. The CAR-Treg of claim 1 or 2, wherein the CAR-Treg expresses FOXP3, CD25, HELIOS, a demethylated Treg-specific demethylated region (TSDR), or a combination thereof.

4. The CAR-Treg of any one of claims 1 to 3, wherein the hinge domain is an IgG4, CD28, or CD8a hinge domain.

5. The CAR-Treg of any one of claims 1 to 4, wherein the transmembrane domain is a CD28 transmembrane domain or a CD8 transmembrane domain.

6. The CAR-Treg of any one of claims 1 to 5, wherein the intracellular domain comprises a signaling domain.

7. The CAR-Treg of claim 6, wherein the signaling domain comprises a CD3 domain.

8. The CAR-Treg of claim 6, wherein the signaling domain comprises a costimulatory domain.

9. The CAR-Treg of claim 8, wherein the signaling domain further comprises a CD3 domain.

10. The CAR-Treg of any one of claims 1 to 5, wherein the intracellular domain does not comprise a signaling domain.

11. The CAR-Treg of claim 7 or 9, wherein the CD3 domain is derived from CD3ζ and the costimulatory domain is derived from the CD28 costimulatory domain.

12. The CAR-Treg of any one of claims 1 to 11, wherein the CAR-Treg does not express an endogenous TCR.

13. The CAR-Treg of any one of claims 1 to 12, wherein the exogenous nucleic acid is integrated into the TCR alpha constant (TRAC) locus of the CAR-Treg.

14. The CAR-Treg of any one of claims 1 to 13, wherein the anti-HLA-A2 antibody is directed to an HLA-A allele selected from A*02:01, A*02:02, A*02:03, A*02:04, A*02:05, A*02:06, A*02:07, A*02:08, A*02:09, A*02:10, A*02:11, A*02:12, A*02:13, A*02:14, A*02:16, A*02:17, A*02:18, A*02:20, A*02:21, A*02:22, A*02:24, A*02:25, A*02:26, A*02:27, A*02:28, A*02:29, A*02:30, A*02:31, A*02:33, A*02:34, A*02:35, A*02:36, A*02:37, A*02:38, A*02:40, A*02:41, A*02:42, A*02:45, A*02:46, A*02:47, A*02:48, A*02:49, A*02:50, A*02:51, A*02:52, A*02:54, A*02:55, A*02:56, A*02:57, A*02:58, A*02:59, A*02:60, A*02:61, A*02:62, A*02:63, A*02:64, A*02:66, A*02:67, A*02:68, A*02:69, A*02:70, A*02:71, A*02:72, A*02:73, A*02:74, A*02:75, A*02:76, A*02:77, A*02:78, A*02:80, A*02:81, A*02:84, A*02:85, A*02:87, A*02:89, A*02:90, A*02:91, A*02:92, A*02:93, A*02:95, A*02:96, A*02:97, A*02:99, A*02:101, A*02:102, A*02:103, A*02:104, A*02:105, A*02:106, A*02:107, A*02:108, A*02:109, A*02:110, A*02:111, A*02:112, A*02:114, A*02:115, A*02:116, A*02:117, A*02:119, A*02:120, A*02:121, A*02:122, A*02:123, A*02:124, A*02:126, A*02:127, A*02:128, A*02:129, A*02:130, A*02:131, A*02:132, A*02:133, A*02:134, A*02:136, A*02:137, A*02:138, A*02:139, A*02:140, A*02:141, A*02:142, A*02:143, A*02:144, A*02:145, A*02:146, A*02:147, A*02:148, A*02:150, A*02:151, A*02:153, A*02:154, A*02:155, A*02:156, A*02:157, A*68:01, A*68:02, A*68:03, A*68:04, A*68:05, A*68:06, A*68:07, A*68:08, A*68:09, A*68:10, A*68:13, A*68:14, A*68:15, A*68:16, A*68:17, A*68:19, A*68:20, A*68:21, A*68:22, A*68:23, A*68:24, A*68:25, A*68:26, A*68:27, A*68:28, A*68:29, A*68:30, A*68:31, A*68:32, A*68:33, A*68:34, A*68:35, A*68:36, A*68:37, A*68:38, A*68:39, A*68:40, A*68:41, A*68:42, A*68:43, A*68:44, A*68:45, A*68:46, A*68:47, A*69:01.

15. The CAR-Treg of claim 14, wherein the anti-HLA-A2 antibody is directed to an eplet of 144TKH contained in an HLA-A allele selected from A*02:01, A*02:02, A*02:03, A*02:04, A*02:05, A*02:06, A*02:07, A*02:08, A*02:09, A*02:10, A*02:11, A*02:12, A*02:13, A*02:14, A*02:16, A*02:17, A*02:18, A*02:20, A*02:21, A*02:22, A*02:24, A*02:25, A*02:26, A*02:27, A*02:28, A*02:29, A*02:30, A*02:31, A*02:33, A*02:34, A*02:35, A*02:36, A*02:37, A*02:38, A*02:40, A*02:41, A*02:42, A*02:45, A*02:46, A*02:47, A*02:48, A*02:49, A*02:50, A*02:51, A*02:52, A*02:54, A*02:55, A*02:56, A*02:57, A*02:58, A*02:59, A*02:60, A*02:61, A*02:62, A*02:63, A*02:64, A*02:66, A*02:67, A*02:68, A*02:69, A*02:70, A*02:71, A*02:72, A*02:73, A*02:74, A*02:75, A*02:76, A*02:77, A*02:78, A*02:80, A*02:81, A*02:84, A*02:85, A*02:87, A*02:89, A*02:90, A*02:91, A*02:92, A*02:93, A*02:95, A*02:96, A*02:97, A*02:99, A*02:101, A*02:102, A*02:103, A*02:104, A*02:105, A*02:106, A*02:107, A*02:108, A*02:109, A*02:110, A*02:111, A*02:112, A*02:114, A*02:115, A*02:116, A*02:117, A*02:119, A*02:120, A*02:121, A*02:122, A*02:123, A*02:124, A*02:126, A*02:127, A*02:128, A*02:129, A*02:130, A*02:131, A*02:132, A*02:133, A*02:134, A*02:136, A*02:137, A*02:138, A*02:139, A*02:140, A*02:141, A*02:142, A*02:143, A*02:144, A*02:145, A*02:146, A*02:147, A*02:148, A*02:150, A*02:151, A*02:153, A*02:154, A*02:155, A*02:156, A*02:157, A*68:01, A*68:02, A*68:03, A*68:04, A*68:05, A*68:06, A*68:07, A*68:08, A*68:09, A*68:10, A*68:13, A*68:14, A*68:15, A*68:16, A*68:17, A*68:19, A*68:20, A*68:21, A*68:22, A*68:23, A*68:24, A*68:25, A*68:26, A*68:27, A*68:28, A*68:29, A*68:30, A*68:31, A*68:32, A*68:33, A*68:34, A*68:35, A*68:36, A*68:37, A*68:38, A*68:39, A*68:40, A*68:41, A*68:42, A*68:43, A*68:44, A*68:45, A*68:46, A*68:47, A*69:01.

16. The CAR-Treg of any one of claims 1 to 15, wherein the CAR-Treg suppresses in vitro proliferation of T cells in the presence of HLA-A2+ cells.

17. The CAR-Treg of any one of claims 1 to 16, wherein the CAR-Treg accumulates in HLA-A2 expressing tissue in vivo.

18. The CAR-Treg of any one of claims 1 to 17, wherein the CAR-Treg suppresses the immune response in the presence of HLA-A2+ and HLA-A28+ cells in vivo.

19. A vector comprising:
an exogenous nucleic acid encoding a chimeric antigen receptor (CAR) comprising the complementarity determining regions (CDRs) of a human anti-HLA-A2 antibody grafted onto an antibody scaffold;
a hinge domain;
a transmembrane domain; and
an intracellular domain.

20. The vector of claim 19, wherein the antibody scaffold is human trastuzumab.

21. The vector of claim 19 or 20, wherein the CAR-Treg expresses FOXP3, CD25, HELIOS or a combination thereof.

22. The vector of any one of claims 19 to 21, wherein the hinge domain is an IgG4, CD28, or CD8a hinge domain.

23. The vector of any one of claims 19 to 22, wherein the intracellular domain comprises a signaling domain.

24. The vector of claim 23, wherein the signaling domain comprises a CD3 domain.

25. The vector of claim 23, wherein the signaling domain comprises a costimulatory domain.

26. The vector of claim 25, wherein the signaling domain further comprises a CD3 domain.

27. The vector of any one of claims 19 to 22, wherein the intracellular domain does not comprise a signaling domain.

28. The vector of claim 24 or 26, wherein the CD3 domain is derived from CD3ζ and the costimulatory domain is derived from the CD28 costimulatory domain.

29. The vector of any one of claims 19 to 28, wherein the vector is a viral vector.

30. The vector of claim 29, wherein the viral vector is a retroviral vector.

31. The vector of claim 30, wherein the retroviral vector is a lentiviral vector.

32. The vector of any one of claims 19 to 31, wherein the vector comprises a nucleic acid that encodes the amino acid sequence of SEQ ID NO:12

33. A method for generating chimeric-antigen-receptor regulatory T cells (CAR-Tregs) comprising:
providing a population of regulatory T cells; and
contacting the population of regulatory T cells with one or more vectors of claims 19-32 under conditions sufficient for transduction of the vector.

34. A method for inducing immune tolerance for organ transplantation comprising:
providing a population of CAR-Tregs of any one of claims 1-18;
administering the population of CAR-Tregs to a subject having received an organ transplant.

35. The method of claim 34, wherein the organ transplant is a non-vascularized transplant.

36. The method of claim 35, wherein the non-vascularized transplant is selected from skin and pancreatic islets.

37. The method of claim 34, wherein the organ transplant is a vascularized transplant.

38. The method of claim 37, wherein the vascularized transplant is selected from heart, lung, kidney, liver, pancreas, small intestine, or composite tissue allograft.

39. The method of claim 34, wherein said administering comprises administering at least 30 million (3×10⁷) to 500 million (5×10⁸) cells to the subject.

40. The method of claim 34, wherein said administration induces immune tolerance to transplanted cells and tissue such that a dose of immunosuppressive drug can be reduced or eliminated.
